SEQUENCE LISTING (452 sequences)

SEQ ID NO: 1 - protein, 9 aa, Artificial sequence
  synthetic sequence
EYNYVVTDH

SEQ ID NO: 2 - protein, 8 aa, Artificial sequence
  synthetic sequence
NKKVVTDH

SEQ ID NO: 3 - protein, 10 aa, Artificial sequence
  synthetic sequence
LEEKKYDYVV

SEQ ID NO: 4 - protein, 9 aa, Artificial sequence
  synthetic sequence
LEEKKYYVV

SEQ ID NO: 5 - protein, 8 aa, Artificial sequence
  synthetic sequence
LEEKVTDH

SEQ ID NO: 6 - protein, 9 aa, Artificial sequence
  synthetic sequence
LEEYVVTDH

SEQ ID NO: 7 - protein, 8 aa, Artificial sequence
  synthetic sequence
ENYVVTDH

SEQ ID NO: 8 - protein, 8 aa, Artificial sequence
  synthetic sequence
EELVVYHD

SEQ ID NO: 9 - protein, 8 aa, Artificial sequence
  synthetic sequence
LEEKKNYV

SEQ ID NO: 10 - protein, 7 aa, Artificial sequence
  synthetic sequence
NYVVTDH

SEQ ID NO: 11 - protein, 10 aa, Artificial sequence
  synthetic sequence
CGALSILEEK

SEQ ID NO: 12 - protein, 8 aa, Artificial sequence
  synthetic sequence
DGYVVTDH

SEQ ID NO: 13 - protein, 9 aa, Artificial sequence
  synthetic sequence
DIGIGLEEK

SEQ ID NO: 14 - protein, 8 aa, Artificial sequence
  synthetic sequence
DLYVVTDH

SEQ ID NO: 15 - protein, 11 aa, Artificial sequence
  synthetic sequence
DSLVVTDHGEK

SEQ ID NO: 16 - protein, 7 aa, Artificial sequence
  synthetic sequence
DYVVTDH

SEQ ID NO: 17 - protein, 12 aa, Artificial sequence
  synthetic sequence
EEKKNGYVVTDH

SEQ ID NO: 18 - protein, 7 aa, Artificial sequence
  synthetic sequence
EMVVTDH

SEQ ID NO: 19 - protein, 9 aa, Artificial sequence
  synthetic sequence
GCTSVVTDH

SEQ ID NO: 20 - protein, 8 aa, Artificial sequence
  synthetic sequence
GDYVVTDH

SEQ ID NO: 21 - protein, 9 aa, Artificial sequence
  synthetic sequence
GGNYVVTDH

SEQ ID NO: 22 - protein, 12 aa, Artificial sequence
  synthetic sequence
GLEEKKNCTGVR

SEQ ID NO: 23 - protein, 11 aa, Artificial sequence
  synthetic sequence
GLEEKMEKHFK

SEQ ID NO: 24 - protein, 11 aa, Artificial sequence
  synthetic sequence
GNEKCTGADSY

SEQ ID NO: 25 - protein, 10 aa, Artificial sequence
  synthetic sequence
GNGVYVVTDH

SEQ ID NO: 26 - protein, 12 aa, Artificial sequence
  synthetic sequence
GNYSYEMEEDHG

SEQ ID NO: 27 - protein, 8 aa, Artificial sequence
  synthetic sequence
GVCVVTDH

SEQ ID NO: 28 - protein, 9 aa, Artificial sequence
  synthetic sequence
GVVYVVTDH

SEQ ID NO: 29 - protein, 12 aa, Artificial sequence
  synthetic sequence
HGSCVRLSIEEK

SEQ ID NO: 30 - protein, 11 aa, Artificial sequence
  synthetic sequence
INATNYVVTDH

SEQ ID NO: 31 - protein, 8 aa, Artificial sequence
  synthetic sequence
KDGYVVTD

SEQ ID NO: 32 - protein, 11 aa, Artificial sequence
  synthetic sequence
KEEGIYVVTDH

SEQ ID NO: 33 - protein, 9 aa, Artificial sequence
  synthetic sequence
KGDYVVTDH

SEQ ID NO: 34 - protein, 11 aa, Artificial sequence
  synthetic sequence
KGGENYVVTDH

SEQ ID NO: 35 - protein, 8 aa, Artificial sequence
  synthetic sequence
KGIGEFKD

SEQ ID NO: 36 - protein, 10 aa, Artificial sequence
  synthetic sequence
KGIGIGEFTS

SEQ ID NO: 37 - protein, 8 aa, Artificial sequence
  synthetic sequence
LESINNYV

SEQ ID NO: 38 - protein, 9 aa, Artificial sequence
  synthetic sequence
LSINYKKGK

SEQ ID NO: 39 - protein, 8 aa, Artificial sequence
  synthetic sequence
MEEKGEKK

SEQ ID NO: 40 - protein, 8 aa, Artificial sequence
  synthetic sequence
NGYVVTDH

SEQ ID NO: 41 - protein, 7 aa, Artificial sequence
  synthetic sequence
NYVVTDA

SEQ ID NO: 42 - protein, 8 aa, Artificial sequence
  synthetic sequence
NYVVTTDH

SEQ ID NO: 43 - protein, 10 aa, Artificial sequence
  synthetic sequence
VVNIKNYVVT

SEQ ID NO: 44 - protein, 9 aa, Artificial sequence
  synthetic sequence
KGNDHVTDH

SEQ ID NO: 45 - protein, 10 aa, Artificial sequence
  synthetic sequence
KGNHFKNCVR

SEQ ID NO: 46 - protein, 9 aa, Artificial sequence
  synthetic sequence
KGNLEIKNI

SEQ ID NO: 47 - protein, 9 aa, Artificial sequence
  synthetic sequence
KKDGYVVTD

SEQ ID NO: 48 - protein, 8 aa, Artificial sequence
  synthetic sequence
KKGNYVVT

SEQ ID NO: 49 - protein, 8 aa, Artificial sequence
  synthetic sequence
KNGYVVTD

SEQ ID NO: 50 - protein, 9 aa, Artificial sequence
  synthetic sequence
KVVTDHGSK

SEQ ID NO: 51 - protein, 13 aa, Artificial sequence
  synthetic sequence
LEEESINNATNIK

SEQ ID NO: 52 - protein, 9 aa, Artificial sequence
  synthetic sequence
LEEGVRKCK

SEQ ID NO: 53 - protein, 9 aa, Artificial sequence
  synthetic sequence
LEEKGKNYV

SEQ ID NO: 54 - protein, 9 aa, Artificial sequence
  synthetic sequence
KGNDHVTDH

SEQ ID NO: 55 - protein, 11 aa, Artificial sequence
  synthetic sequence
LEEKGKNYVVT

SEQ ID NO: 56 - protein, 13 aa, Artificial sequence
  synthetic sequence
LEEKGKNYVVTDH

SEQ ID NO: 57 - protein, 12 aa, Artificial sequence
  synthetic sequence
LEEKIGEYVVTD

SEQ ID NO: 58 - protein, 13 aa, Artificial sequence
  synthetic sequence
LEEKIGEYVVTDH

SEQ ID NO: 59 - protein, 7 aa, Artificial sequence
  synthetic sequence
LEEKKGI

SEQ ID NO: 60 - protein, 12 aa, Artificial sequence
  synthetic sequence
LEEKKGISGDLR

SEQ ID NO: 61 - protein, 9 aa, Artificial sequence
  synthetic sequence
LEEKKVCCK

SEQ ID NO: 62 - protein, 10 aa, Artificial sequence
  synthetic sequence
LEEKKVCGVR

SEQ ID NO: 63 - protein, 11 aa, Artificial sequence
  synthetic sequence
LEEKNATNIKN

SEQ ID NO: 64 - protein, 11 aa, Artificial sequence
  synthetic sequence
LEEKTSISNIK

SEQ ID NO: 65 - protein, 10 aa, Artificial sequence
  synthetic sequence
LEEKVCGVRK

SEQ ID NO: 66 - protein, 12 aa, Artificial sequence
  synthetic sequence
LEEVVMEEDGVR

SEQ ID NO: 67 - protein, 13 aa, Artificial sequence
  synthetic sequence
LEKKEGNYVVTDH

SEQ ID NO: 68 - protein, 12 aa, Artificial sequence
  synthetic sequence
LEKKNGYVVTDH

SEQ ID NO: 69 - protein, 13 aa, Artificial sequence
  synthetic sequence
LELEVTDYVVTDH

SEQ ID NO: 70 - protein, 8 aa, Artificial sequence
  synthetic sequence
NYVVVTDH

SEQ ID NO: 71 - protein, 11 aa, Artificial sequence
  synthetic sequence
SLSINKGNYEK

SEQ ID NO: 72 - protein, 9 aa, Artificial sequence
  synthetic sequence
SLSINNYVV

SEQ ID NO: 73 - protein, 9 aa, Artificial sequence
  synthetic sequence
TDIGEFLEK

SEQ ID NO: 74 - protein, 9 aa, Artificial sequence
  synthetic sequence
TSIGVKKGG

SEQ ID NO: 75 - protein, 12 aa, Artificial sequence
  synthetic sequence
TSIVTDHGSCVR

SEQ ID NO: 76 - protein, 11 aa, Artificial sequence
  synthetic sequence
ADSFVIGDEVR

SEQ ID NO: 77 - protein, 9 aa, Artificial sequence
  synthetic sequence
APGQTGKIA

SEQ ID NO: 78 - protein, 10 aa, Artificial sequence
  synthetic sequence
DSFVIRGDEV

SEQ ID NO: 79 - protein, 10 aa, Artificial sequence
  synthetic sequence
DSKVGGNYNY

SEQ ID NO: 80 - protein, 10 aa, Artificial sequence
  synthetic sequence
GFQPTNGVGY

SEQ ID NO: 81 - protein, 9 aa, Artificial sequence
  synthetic sequence
GVGYQPYRV

SEQ ID NO: 82 - protein, 10 aa, Artificial sequence
  synthetic sequence
GVGYQPYRVV

SEQ ID NO: 83 - protein, 9 aa, Artificial sequence
  synthetic sequence
IAPGQTGKI

SEQ ID NO: 84 - protein, 11 aa, Artificial sequence
  synthetic sequence
IAPGQTGKIAD

SEQ ID NO: 85 - protein, 8 aa, Artificial sequence
  synthetic sequence
KVGGNYNY

SEQ ID NO: 86 - protein, 9 aa, Artificial sequence
  synthetic sequence
KVGGNYNYL

SEQ ID NO: 87 - protein, 11 aa, Artificial sequence
  synthetic sequence
NDSFVIRGDEV

SEQ ID NO: 88 - protein, 11 aa, Artificial sequence
  synthetic sequence
NLDSKVGGNYN

SEQ ID NO: 89 - protein, 12 aa, Artificial sequence
  synthetic sequence
NLDSKVGGNYNY

SEQ ID NO: 90 - protein, 11 aa, Artificial sequence
  synthetic sequence
NPGQTGKIADY

SEQ ID NO: 91 - protein, 8 aa, Artificial sequence
  synthetic sequence
NYKLPDDF

SEQ ID NO: 92 - protein, 8 aa, Artificial sequence
  synthetic sequence
RDISTEIY

SEQ ID NO: 93 - protein, 9 aa, Artificial sequence
  synthetic sequence
RDISTEIYQ

SEQ ID NO: 94 - protein, 9 aa, Artificial sequence
  synthetic sequence
SKVGGNYNY

SEQ ID NO: 95 - protein, 8 aa, Artificial sequence
  synthetic sequence
VGYQPYRV

SEQ ID NO: 96 - protein, 12 aa, Artificial sequence
  synthetic sequence
VQPTESIPFGEK

SEQ ID NO: 97 - protein, 11 aa, Artificial sequence
  synthetic sequence
ADSFVIRGDEV

SEQ ID NO: 98 - protein, 8 aa, Artificial sequence
  synthetic sequence
ADYSVLYN

SEQ ID NO: 99 - protein, 11 aa, Artificial sequence
  synthetic sequence
APGQTGKIADY

SEQ ID NO: 100 - protein, 9 aa, Artificial sequence
  synthetic sequence
DISTEIYQA

SEQ ID NO: 101 - protein, 9 aa, Artificial sequence
  synthetic sequence
DSKVGGNYN

SEQ ID NO: 102 - protein, 9 aa, Artificial sequence
  synthetic sequence
ELLHAPATV

SEQ ID NO: 103 - protein, 11 aa, Artificial sequence
  synthetic sequence
DSKVGGNYNYL

SEQ ID NO: 104 - protein, 11 aa, Artificial sequence
  synthetic sequence
GDEVRQIAPGQ

SEQ ID NO: 105 - protein, 10 aa, Artificial sequence
  synthetic sequence
RDISTEIYQA

SEQ ID NO: 106 - protein, 9 aa, Artificial sequence
  synthetic sequence
RQIAPGQTG

SEQ ID NO: 107 - protein, 11 aa, Artificial sequence
  synthetic sequence
RQIAPGQTGKI

SEQ ID NO: 108 - protein, 9 aa, Artificial sequence
  synthetic sequence
SFVIRGDEV

SEQ ID NO: 109 - protein, 8 aa, Artificial sequence
  synthetic sequence
SPTKLNDL

SEQ ID NO: 110 - protein, 9 aa, Artificial sequence
  synthetic sequence
TNLCPFRIS

SEQ ID NO: 111 - protein, 9 aa, Artificial sequence
  synthetic sequence
VGYQPYRVV

SEQ ID NO: 112 - protein, 10 aa, Artificial sequence
  synthetic sequence
VIRGDEVRQI

SEQ ID NO: 113 - protein, 11 aa, Artificial sequence
  synthetic sequence
VQPTLYFSTFK

SEQ ID NO: 114 - protein, 8 aa, Artificial sequence
  synthetic sequence
YADSFVIR

SEQ ID NO: 115 - protein, 9 aa, Artificial sequence
  synthetic sequence
YGFQPTNGV

SEQ ID NO: 116 - protein, 11 aa, Artificial sequence
  synthetic sequence
YGFQPTNGVGY

SEQ ID NO: 117 - protein, 8 aa, Artificial sequence
  synthetic sequence
YGVSPTKL

SEQ ID NO: 118 - protein, 10 aa, Artificial sequence
  synthetic sequence
AAGYLQPRTF

SEQ ID NO: 119 - protein, 11 aa, Artificial sequence
  synthetic sequence
ASTGSNVFQTR

SEQ ID NO: 120 - protein, 8 aa, Artificial sequence
  synthetic sequence
ASVSPTKL

SEQ ID NO: 121 - protein, 9 aa, Artificial sequence
  synthetic sequence
AVLYQGVNC

SEQ ID NO: 122 - protein, 11 aa, Artificial sequence
  synthetic sequence
AVLYQGVNCTE

SEQ ID NO: 123 - protein, 12 aa, Artificial sequence
  synthetic sequence
AVLYQGVNCTEV

SEQ ID NO: 124 - protein, 8 aa, Artificial sequence
  synthetic sequence
AVRDPQTI

SEQ ID NO: 125 - protein, 12 aa, Artificial sequence
  synthetic sequence
DASYQTQTNSPR

SEQ ID NO: 126 - protein, 9 aa, Artificial sequence
  synthetic sequence
DATNVVIKV

SEQ ID NO: 127 - protein, 8 aa, Artificial sequence
  synthetic sequence
DIITPCSF

SEQ ID NO: 128 - protein, 12 aa, Artificial sequence
  synthetic sequence
DLEGKQGNFKIN

SEQ ID NO: 129 - protein, 12 aa, Artificial sequence
  synthetic sequence
DLEGKQGNFKNI

SEQ ID NO: 130 - protein, 10 aa, Artificial sequence
  synthetic sequence
DPLSETKCTI

SEQ ID NO: 131 - protein, 12 aa, Artificial sequence
  synthetic sequence
DSKVGGNYNYYR

SEQ ID NO: 132 - protein, 10 aa, Artificial sequence
  synthetic sequence
DTGSNVFQTR

SEQ ID NO: 133 - protein, 9 aa, Artificial sequence
  synthetic sequence
DTSNQVAVL

SEQ ID NO: 134 - protein, 12 aa, Artificial sequence
  synthetic sequence
DVSVITPGTNTS

SEQ ID NO: 135 - protein, 9 aa, Artificial sequence
  synthetic sequence
EEHVNNSYE

SEQ ID NO: 136 - protein, 9 aa, Artificial sequence
  synthetic sequence
ERDISTIYQ

SEQ ID NO: 137 - protein, 11 aa, Artificial sequence
  synthetic sequence
ESTGSNVFQTR

SEQ ID NO: 138 - protein, 12 aa, Artificial sequence
  synthetic sequence
EVPVAIHADAGI

SEQ ID NO: 139 - protein, 8 aa, Artificial sequence
  synthetic sequence
FAGVSPTK

SEQ ID NO: 140 - protein, 7 aa, Artificial sequence
  synthetic sequence
FASVLYN

SEQ ID NO: 141 - protein, 9 aa, Artificial sequence
  synthetic sequence
GETRFASVY

SEQ ID NO: 142 - protein, 7 aa, Artificial sequence
  synthetic sequence
GPDKVFR

SEQ ID NO: 143 - protein, 9 aa, Artificial sequence
  synthetic sequence
GRPQTLEIL

SEQ ID NO: 144 - protein, 9 aa, Artificial sequence
  synthetic sequence
GSNVFQTAR

SEQ ID NO: 145 - protein, 7 aa, Artificial sequence
  synthetic sequence
GTQDLFL

SEQ ID NO: 146 - protein, 11 aa, Artificial sequence
  synthetic sequence
GTTLDSKTQSI

SEQ ID NO: 147 - protein, 10 aa, Artificial sequence
  synthetic sequence
GVGYQPLHAP

SEQ ID NO: 148 - protein, 12 aa, Artificial sequence
  synthetic sequence
GVNCTEVPVAIH

SEQ ID NO: 149 - protein, 8 aa, Artificial sequence
  synthetic sequence
GVSPTKLL

SEQ ID NO: 150 - protein, 12 aa, Artificial sequence
  synthetic sequence
GVTPGTNTSNQV

SEQ ID NO: 151 - protein, 10 aa, Artificial sequence
  synthetic sequence
IADSNNLDSK

SEQ ID NO: 152 - protein, 10 aa, Artificial sequence
  synthetic sequence
IGAEIPVASQ

SEQ ID NO: 153 - protein, 11 aa, Artificial sequence
  synthetic sequence
IGAEPIGASVA

SEQ ID NO: 154 - protein, 8 aa, Artificial sequence
  synthetic sequence
ISNSFSTF

SEQ ID NO: 155 - protein, 9 aa, Artificial sequence
  synthetic sequence
ISNSFSTFK

SEQ ID NO: 156 - protein, 11 aa, Artificial sequence
  synthetic sequence
ITPGTNTSQDV

SEQ ID NO: 157 - protein, 7 aa, Artificial sequence
  synthetic sequence
KLFVIFT

SEQ ID NO: 158 - protein, 7 aa, Artificial sequence
  synthetic sequence
KSNIVVI

SEQ ID NO: 159 - protein, 8 aa, Artificial sequence
  synthetic sequence
LDISTEIY

SEQ ID NO: 160 - protein, 10 aa, Artificial sequence
  synthetic sequence
LDISTEIYQA

SEQ ID NO: 161 - protein, 7 aa, Artificial sequence
  synthetic sequence
LLIVNIK

SEQ ID NO: 162 - protein, 7 aa, Artificial sequence
  synthetic sequence
LLSGEVF

SEQ ID NO: 163 - protein, 7 aa, Artificial sequence
  synthetic sequence
LSIGEVF

SEQ ID NO: 164 - protein, 13 aa, Artificial sequence
  synthetic sequence
LTDIADTTDAVRD

SEQ ID NO: 165 - protein, 10 aa, Artificial sequence
  synthetic sequence
LTTRTQPPAY

SEQ ID NO: 166 - protein, 7 aa, Artificial sequence
  synthetic sequence
LTVGEVF

SEQ ID NO: 167 - protein, 7 aa, Artificial sequence
  synthetic sequence
LVDLPIR

SEQ ID NO: 168 - protein, 7 aa, Artificial sequence
  synthetic sequence
LVDLQTL

SEQ ID NO: 169 - protein, 7 aa, Artificial sequence
  synthetic sequence
NDSFVIR

SEQ ID NO: 170 - protein, 10 aa, Artificial sequence
  synthetic sequence
NGLLHAPATV

SEQ ID NO: 171 - protein, 14 aa, Artificial sequence
  synthetic sequence
NGLTGITPGTNTSN

SEQ ID NO: 172 - protein, 8 aa, Artificial sequence
  synthetic sequence
NIDINLVR

SEQ ID NO: 173 - protein, 8 aa, Artificial sequence
  synthetic sequence
NIDTPINL

SEQ ID NO: 174 - protein, 10 aa, Artificial sequence
  synthetic sequence
NLDPLSETKC

SEQ ID NO: 175 - protein, 8 aa, Artificial sequence
  synthetic sequence
NLREDLPI

SEQ ID NO: 176 - protein, 8 aa, Artificial sequence
  synthetic sequence
NLREEPLV

SEQ ID NO: 177 - protein, 13 aa, Artificial sequence
  synthetic sequence
NLTTRTQLPSSVL

SEQ ID NO: 178 - protein, 11 aa, Artificial sequence
  synthetic sequence
NLVFRSDGVYF

SEQ ID NO: 179 - protein, 7 aa, Artificial sequence
  synthetic sequence
NPVLPIR

SEQ ID NO: 180 - protein, 8 aa, Artificial sequence
  synthetic sequence
NSNNLSNL

SEQ ID NO: 181 - protein, 9 aa, Artificial sequence
  synthetic sequence
NSNNLSNLK

SEQ ID NO: 182 - protein, 8 aa, Artificial sequence
  synthetic sequence
NVRDPQTL

SEQ ID NO: 183 - protein, 10 aa, Artificial sequence
  synthetic sequence
PGTNTSNQPV

SEQ ID NO: 184 - protein, 10 aa, Artificial sequence
  synthetic sequence
PVAIHADQLV

SEQ ID NO: 185 - protein, 8 aa, Artificial sequence
  synthetic sequence
PVAIHADV

SEQ ID NO: 186 - protein, 10 aa, Artificial sequence
  synthetic sequence
QEHVNNSYEC

SEQ ID NO: 187 - protein, 7 aa, Artificial sequence
  synthetic sequence
QGSNVFQ

SEQ ID NO: 188 - protein, 9 aa, Artificial sequence
  synthetic sequence
QGSNVFQTR

SEQ ID NO: 189 - protein, 12 aa, Artificial sequence
  synthetic sequence
QGVNCTEVPVAI

SEQ ID NO: 190 - protein, 13 aa, Artificial sequence
  synthetic sequence
QGVNCTEVPVAIH

SEQ ID NO: 191 - protein, 14 aa, Artificial sequence
  synthetic sequence
QGVNCTEVPVAIHA

SEQ ID NO: 192 - protein, 8 aa, Artificial sequence
  synthetic sequence
QLPIGINI

SEQ ID NO: 193 - protein, 9 aa, Artificial sequence
  synthetic sequence
QQSVITPGT

SEQ ID NO: 194 - protein, 10 aa, Artificial sequence
  synthetic sequence
QQSVITPGTN

SEQ ID NO: 195 - protein, 7 aa, Artificial sequence
  synthetic sequence
QSLLVVI

SEQ ID NO: 196 - protein, 8 aa, Artificial sequence
  synthetic sequence
QSTGSNVF

SEQ ID NO: 197 - protein, 11 aa, Artificial sequence
  synthetic sequence
QSTGSNVFQTR

SEQ ID NO: 198 - protein, 7 aa, Artificial sequence
  synthetic sequence
QTGKIAF

SEQ ID NO: 199 - protein, 13 aa, Artificial sequence
  synthetic sequence
QTITPGTNTSNQV

SEQ ID NO: 200 - protein, 8 aa, Artificial sequence
  synthetic sequence
QTPGTNTN

SEQ ID NO: 201 - protein, 10 aa, Artificial sequence
  synthetic sequence
QTQTNSPRAR

SEQ ID NO: 202 - protein, 10 aa, Artificial sequence
  synthetic sequence
SCDIPIGAGI

SEQ ID NO: 203 - protein, 13 aa, Artificial sequence
  synthetic sequence
SFGGVSVITPGTD

SEQ ID NO: 204 - protein, 9 aa, Artificial sequence
  synthetic sequence
SFVIGDEVR

SEQ ID NO: 205 - protein, 10 aa, Artificial sequence
  synthetic sequence
SGVEKGIYQT

SEQ ID NO: 206 - protein, 11 aa, Artificial sequence
  synthetic sequence
SGVEKGIYQTS

SEQ ID NO: 207 - protein, 10 aa, Artificial sequence
  synthetic sequence
SIVNNATNVV

SEQ ID NO: 208 - protein, 9 aa, Artificial sequence
  synthetic sequence
SKTPINLVR

SEQ ID NO: 209 - protein, 12 aa, Artificial sequence
  synthetic sequence
SLEKGIYQTSNF

SEQ ID NO: 210 - protein, 8 aa, Artificial sequence
  synthetic sequence
SNIIRTLD

SEQ ID NO: 211 - protein, 7 aa, Artificial sequence
  synthetic sequence
SSEFRVY

SEQ ID NO: 212 - protein, 10 aa, Artificial sequence
  synthetic sequence
STGSNVFQHH

SEQ ID NO: 213 - protein, 11 aa, Artificial sequence
  synthetic sequence
STGSNVFQTAR

SEQ ID NO: 214 - protein, 9 aa, Artificial sequence
  synthetic sequence
SYQTQTNSH

SEQ ID NO: 215 - protein, 8 aa, Artificial sequence
  synthetic sequence
SYVSQPFL

SEQ ID NO: 216 - protein, 9 aa, Artificial sequence
  synthetic sequence
SYVSQPFLM

SEQ ID NO: 217 - protein, 11 aa, Artificial sequence
  synthetic sequence
TESNVRDPQTL

SEQ ID NO: 218 - protein, 10 aa, Artificial sequence
  synthetic sequence
TEVPVAIHAN

SEQ ID NO: 219 - protein, 10 aa, Artificial sequence
  synthetic sequence
TGSNVFQTAR

SEQ ID NO: 220 - protein, 8 aa, Artificial sequence
  synthetic sequence
TGSNVFTR

SEQ ID NO: 221 - protein, 9 aa, Artificial sequence
  synthetic sequence
TKADSFVIR

SEQ ID NO: 222 - protein, 11 aa, Artificial sequence
  synthetic sequence
TLPGTNTSNQV

SEQ ID NO: 223 - protein, 12 aa, Artificial sequence
  synthetic sequence
TPGNTSNQVAVL

SEQ ID NO: 224 - protein, 7 aa, Artificial sequence
  synthetic sequence
TPGTAVL

SEQ ID NO: 225 - protein, 10 aa, Artificial sequence
  synthetic sequence
TPGTNTAVLY

SEQ ID NO: 226 - protein, 9 aa, Artificial sequence
  synthetic sequence
TQLPHSTQD

SEQ ID NO: 227 - protein, 10 aa, Artificial sequence
  synthetic sequence
TQLPPAHVSG

SEQ ID NO: 228 - protein, 9 aa, Artificial sequence
  synthetic sequence
TQLPPAYTD

SEQ ID NO: 229 - protein, 8 aa, Artificial sequence
  synthetic sequence
TQLPSSVL

SEQ ID NO: 230 - protein, 8 aa, Artificial sequence
  synthetic sequence
TQSLLIQF

SEQ ID NO: 231 - protein, 9 aa, Artificial sequence
  synthetic sequence
TQSLLIVNI

SEQ ID NO: 232 - protein, 10 aa, Artificial sequence
  synthetic sequence
TQSLLIVNIK

SEQ ID NO: 233 - protein, 7 aa, Artificial sequence
  synthetic sequence
TQSLLVY

SEQ ID NO: 234 - protein, 7 aa, Artificial sequence
  synthetic sequence
TSFTVEK

SEQ ID NO: 235 - protein, 8 aa, Artificial sequence
  synthetic sequence
TWSNVFQT

SEQ ID NO: 236 - protein, 9 aa, Artificial sequence
  synthetic sequence
TWSNVFQTR

SEQ ID NO: 237 - protein, 11 aa, Artificial sequence
  synthetic sequence
TWSNVFQTRAG

SEQ ID NO: 238 - protein, 12 aa, Artificial sequence
  synthetic sequence
TWSNVFQTRAGC

SEQ ID NO: 239 - protein, 8 aa, Artificial sequence
  synthetic sequence
VAIGSNVF

SEQ ID NO: 240 - protein, 9 aa, Artificial sequence
  synthetic sequence
VAPGQTGKI

SEQ ID NO: 241 - protein, 10 aa, Artificial sequence
  synthetic sequence
VCGVDTTDAV

SEQ ID NO: 242 - protein, 10 aa, Artificial sequence
  synthetic sequence
VGGNYNYLLK

SEQ ID NO: 243 - protein, 10 aa, Artificial sequence
  synthetic sequence
VGKSNLKPFE

SEQ ID NO: 244 - protein, 13 aa, Artificial sequence
  synthetic sequence
VNLTTPAYTNSFT

SEQ ID NO: 245 - protein, 11 aa, Artificial sequence
  synthetic sequence
VNLTTPFFSNV

SEQ ID NO: 246 - protein, 11 aa, Artificial sequence
  synthetic sequence
VPVAIHADAGI

SEQ ID NO: 247 - protein, 8 aa, Artificial sequence
  synthetic sequence
VQPTSVTF

SEQ ID NO: 248 - protein, 10 aa, Artificial sequence
  synthetic sequence
VSPTKLNDLF

SEQ ID NO: 249 - protein, 9 aa, Artificial sequence
  synthetic sequence
WIGAEHVNN

SEQ ID NO: 250 - protein, 11 aa, Artificial sequence
  synthetic sequence
WSTGSNVFQTR

SEQ ID NO: 251 - protein, 12 aa, Artificial sequence
  synthetic sequence
YADSFVIGDEVR

SEQ ID NO: 252 - protein, 13 aa, Artificial sequence
  synthetic sequence
YQGVNCTEVPVAI

SEQ ID NO: 253 - protein, 14 aa, Artificial sequence
  synthetic sequence
YQGVNCTEVPVAIH

SEQ ID NO: 254 - protein, 8 aa, Artificial sequence
  synthetic sequence
NKDGIIWV

SEQ ID NO: 255 - protein, 8 aa, Artificial sequence
  synthetic sequence
NSTPGSSR

SEQ ID NO: 256 - protein, 8 aa, Artificial sequence
  synthetic sequence
VTPSGTWL

SEQ ID NO: 257 - protein, 9 aa, Artificial sequence
  synthetic sequence
QRNAPRITF

SEQ ID NO: 258 - protein, 9 aa, Artificial sequence
  synthetic sequence
GLPYGANKD

SEQ ID NO: 259 - protein, 9 aa, Artificial sequence
  synthetic sequence
ANKDGIIWV

SEQ ID NO: 260 - protein, 9 aa, Artificial sequence
  synthetic sequence
HIGTRNPAN

SEQ ID NO: 261 - protein, 9 aa, Artificial sequence
  synthetic sequence
PANNAAIVL

SEQ ID NO: 262 - protein, 9 aa, Artificial sequence
  synthetic sequence
LDRLNQLES

SEQ ID NO: 263 - protein, 9 aa, Artificial sequence
  synthetic sequence
QQGQTVTKK

SEQ ID NO: 264 - protein, 9 aa, Artificial sequence
  synthetic sequence
GDQELIRQG

SEQ ID NO: 265 - protein, 9 aa, Artificial sequence
  synthetic sequence
TDYKHWPQI

SEQ ID NO: 266 - protein, 9 aa, Artificial sequence
  synthetic sequence
EVTPSGTWL

SEQ ID NO: 267 - protein, 9 aa, Artificial sequence
  synthetic sequence
KTFPPTEPK

SEQ ID NO: 268 - protein, 9 aa, Artificial sequence
  synthetic sequence
QTVTLLPAA

SEQ ID NO: 269 - protein, 9 aa, Artificial sequence
  synthetic sequence
DLDDFSKQL

SEQ ID NO: 270 - protein, 10 aa, Artificial sequence
  synthetic sequence
RPQGLPNNTA

SEQ ID NO: 271 - protein, 10 aa, Artificial sequence
  synthetic sequence
TQHGKEDLKF

SEQ ID NO: 272 - protein, 10 aa, Artificial sequence
  synthetic sequence
HIGTRNPANN

SEQ ID NO: 273 - protein, 10 aa, Artificial sequence
  synthetic sequence
QQGQTVTKKS

SEQ ID NO: 274 - protein, 10 aa, Artificial sequence
  synthetic sequence
GRRGPEQTQG

SEQ ID NO: 275 - protein, 10 aa, Artificial sequence
  synthetic sequence
RGPEQTQGNF

SEQ ID NO: 276 - protein, 10 aa, Artificial sequence
  synthetic sequence
QGNFGDQELI

SEQ ID NO: 277 - protein, 10 aa, Artificial sequence
  synthetic sequence
QELIRQGTDY

SEQ ID NO: 278 - protein, 10 aa, Artificial sequence
  synthetic sequence
GTDYKHWPQI

SEQ ID NO: 279 - protein, 10 aa, Artificial sequence
  synthetic sequence
TDYKHWPQIA

SEQ ID NO: 280 - protein, 10 aa, Artificial sequence
  synthetic sequence
MEVTPSGTWL

SEQ ID NO: 281 - protein, 10 aa, Artificial sequence
  synthetic sequence
KDPNFKDQVI

SEQ ID NO: 282 - protein, 10 aa, Artificial sequence
  synthetic sequence
PNFKDQVILL

SEQ ID NO: 283 - protein, 11 aa, Artificial sequence
  synthetic sequence
KFPRGQGVPIN

SEQ ID NO: 284 - protein, 11 aa, Artificial sequence
  synthetic sequence
HIGTRNPANNA

SEQ ID NO: 285 - protein, 11 aa, Artificial sequence
  synthetic sequence
RNPANNAAIVL

SEQ ID NO: 286 - protein, 11 aa, Artificial sequence
  synthetic sequence
PQGTTLPKGFY

SEQ ID NO: 287 - protein, 11 aa, Artificial sequence
  synthetic sequence
GNGGDAALALL

SEQ ID NO: 288 - protein, 11 aa, Artificial sequence
  synthetic sequence
LLLLDRLNQLE

SEQ ID NO: 289 - protein, 11 aa, Artificial sequence
  synthetic sequence
QQGQTVTKKSA

SEQ ID NO: 290 - protein, 11 aa, Artificial sequence
  synthetic sequence
RGPEQTQGNFG

SEQ ID NO: 291 - protein, 11 aa, Artificial sequence
  synthetic sequence
QGTDYKHWPQI

SEQ ID NO: 292 - protein, 11 aa, Artificial sequence
  synthetic sequence
DDKDPNFKDQV

SEQ ID NO: 293 - protein, 11 aa, Artificial sequence
  synthetic sequence
DETQALPQRQK

SEQ ID NO: 294 - protein, 11 aa, Artificial sequence
  synthetic sequence
KQQTVTLLPAA

SEQ ID NO: 295 - protein, 11 aa, Artificial sequence
  synthetic sequence
DLDDFSKQLQQ

SEQ ID NO: 296 - protein, 12 aa, Artificial sequence
  synthetic sequence
NGPQNQRNAPRI

SEQ ID NO: 297 - protein, 12 aa, Artificial sequence
  synthetic sequence
ALNTPKDHIGTR

SEQ ID NO: 298 - protein, 12 aa, Artificial sequence
  synthetic sequence
PKDHIGTRNPAN

SEQ ID NO: 299 - protein, 12 aa, Artificial sequence
  synthetic sequence
QLPQGTTLPKGF

SEQ ID NO: 300 - protein, 12 aa, Artificial sequence
  synthetic sequence
NSSRNSTPGSSR

SEQ ID NO: 301 - protein, 12 aa, Artificial sequence
  synthetic sequence
GNGGDAALALLL

SEQ ID NO: 302 - protein, 12 aa, Artificial sequence
  synthetic sequence
NQLESKMSGKGQ

SEQ ID NO: 303 - protein, 12 aa, Artificial sequence
  synthetic sequence
GRRGPEQTQGNF

SEQ ID NO: 304 - protein, 12 aa, Artificial sequence
  synthetic sequence
RGPEQTQGNFGD

SEQ ID NO: 305 - protein, 12 aa, Artificial sequence
  synthetic sequence
RQGTDYKHWPQI

SEQ ID NO: 306 - protein, 12 aa, Artificial sequence
  synthetic sequence
KDPNFKDQVILL

SEQ ID NO: 307 - protein, 12 aa, Artificial sequence
  synthetic sequence
ADETQALPQRQK

SEQ ID NO: 308 - protein, 12 aa, Artificial sequence
  synthetic sequence
LDDFSKQLQQSM

SEQ ID NO: 309 - protein, 15 aa, Artificial sequence
  synthetic sequence
SGGGGGGAEPGQALF

SEQ ID NO: 310 - protein, 14 aa, Artificial sequence
  synthetic sequence
ASMDTVTSSSSSSL

SEQ ID NO: 311 - protein, 14 aa, Artificial sequence
  synthetic sequence
NVTAPTPQQLQAFK

SEQ ID NO: 312 - protein, 14 aa, Artificial sequence
  synthetic sequence
SIGPQILTSPSPSK

SEQ ID NO: 313 - protein, 13 aa, Artificial sequence
  synthetic sequence
GYLSPDLSKVRSN

SEQ ID NO: 314 - protein, 13 aa, Artificial sequence
  synthetic sequence
HVEVLENVPLTTH

SEQ ID NO: 315 - protein, 13 aa, Artificial sequence
  synthetic sequence
NVTAPTPQQLQAF

SEQ ID NO: 316 - protein, 12 aa, Artificial sequence
  synthetic sequence
GDFGLATEKSRW

SEQ ID NO: 317 - protein, 12 aa, Artificial sequence
  synthetic sequence
GGEHNPPSIYLE

SEQ ID NO: 318 - protein, 12 aa, Artificial sequence
  synthetic sequence
PKIHRSASEPSL

SEQ ID NO: 319 - protein, 12 aa, Artificial sequence
  synthetic sequence
RGYLSPDLSKVR

SEQ ID NO: 320 - protein, 12 aa, Artificial sequence
  synthetic sequence
SIPIPQPFRPAD

SEQ ID NO: 321 - protein, 12 aa, Artificial sequence
  synthetic sequence
SNPKSPQKPIVR

SEQ ID NO: 322 - protein, 12 aa, Artificial sequence
  synthetic sequence
SSSSLSVLPSSL

SEQ ID NO: 323 - protein, 12 aa, Artificial sequence
  synthetic sequence
VLENVPLTTHNF

SEQ ID NO: 324 - protein, 11 aa, Artificial sequence
  synthetic sequence
DTVTSSSSSSL

SEQ ID NO: 325 - protein, 11 aa, Artificial sequence
  synthetic sequence
FQNPTDVARSN

SEQ ID NO: 326 - protein, 11 aa, Artificial sequence
  synthetic sequence
HHPIPQEEASL

SEQ ID NO: 327 - protein, 11 aa, Artificial sequence
  synthetic sequence
KGKWHGDVAVK

SEQ ID NO: 328 - protein, 11 aa, Artificial sequence
  synthetic sequence
LSPDLSKVRSN

SEQ ID NO: 329 - protein, 11 aa, Artificial sequence
  synthetic sequence
NINNRDQIIFM

SEQ ID NO: 330 - protein, 11 aa, Artificial sequence
  synthetic sequence
PSLNRAGFQTE

SEQ ID NO: 331 - protein, 11 aa, Artificial sequence
  synthetic sequence
RIGSGSFGTVY

SEQ ID NO: 332 - protein, 11 aa, Artificial sequence
  synthetic sequence
TAPTPQQLQAF

SEQ ID NO: 333 - protein, 11 aa, Artificial sequence
  synthetic sequence
TVVPARCGVTV

SEQ ID NO: 334 - protein, 10 aa, Artificial sequence
  synthetic sequence
ALQQREQQLL

SEQ ID NO: 335 - protein, 10 aa, Artificial sequence
  synthetic sequence
ASIELLARSL

SEQ ID NO: 336 - protein, 10 aa, Artificial sequence
  synthetic sequence
DQLDLLFVSK

SEQ ID NO: 337 - protein, 10 aa, Artificial sequence
  synthetic sequence
GLSATPPASL

SEQ ID NO: 338 - protein, 10 aa, Artificial sequence
  synthetic sequence
GNGTDFSVSS

SEQ ID NO: 339 - protein, 10 aa, Artificial sequence
  synthetic sequence
IGDFGLATEK

SEQ ID NO: 340 - protein, 10 aa, Artificial sequence
  synthetic sequence
INTIEPVNID

SEQ ID NO: 341 - protein, 10 aa, Artificial sequence
  synthetic sequence
NNRDQIIFMV

SEQ ID NO: 342 - protein, 10 aa, Artificial sequence
  synthetic sequence
NVPLTTHNFV

SEQ ID NO: 343 - protein, 10 aa, Artificial sequence
  synthetic sequence
QAFKNEVGVL

SEQ ID NO: 344 - protein, 10 aa, Artificial sequence
  synthetic sequence
QKSPGPQRER

SEQ ID NO: 345 - protein, 10 aa, Artificial sequence
  synthetic sequence
SSLSVLPSSL

SEQ ID NO: 346 - protein, 10 aa, Artificial sequence
  synthetic sequence
STKPQLAIVT

SEQ ID NO: 347 - protein, 10 aa, Artificial sequence
  synthetic sequence
SVFQNPTDVA

SEQ ID NO: 348 - protein, 10 aa, Artificial sequence
  synthetic sequence
TGEELHVEVL

SEQ ID NO: 349 - protein, 10 aa, Artificial sequence
  synthetic sequence
VLENVPLTTH

SEQ ID NO: 350 - protein, 9 aa, Artificial sequence
  synthetic sequence
GDFGLATEK

SEQ ID NO: 351 - protein, 9 aa, Artificial sequence
  synthetic sequence
KLIDIARQT

SEQ ID NO: 352 - protein, 9 aa, Artificial sequence
  synthetic sequence
LSPDLSKVR

SEQ ID NO: 353 - protein, 9 aa, Artificial sequence
  synthetic sequence
SIELLARSL

SEQ ID NO: 354 - protein, 9 aa, Artificial sequence
  synthetic sequence
TSPSPSKSI

SEQ ID NO: 355 - protein, 9 aa, Artificial sequence
  synthetic sequence
TVKIGDFGL

SEQ ID NO: 356 - protein, 8 aa, Artificial sequence
  synthetic sequence
ENVPLTTH

SEQ ID NO: 357 - protein, 8 aa, Artificial sequence
  synthetic sequence
KNEVGVLR

SEQ ID NO: 358 - protein, 8 aa, Artificial sequence
  synthetic sequence
LTSPSPSK

SEQ ID NO: 359 - protein, 8 aa, Artificial sequence
  synthetic sequence
MAPEVIRM

SEQ ID NO: 360 - protein, 8 aa, Artificial sequence
  synthetic sequence
NRDQIIFM

SEQ ID NO: 361 - protein, 8 aa, Artificial sequence
  synthetic sequence
RGYLSPDL

SEQ ID NO: 362 - protein, 8 aa, Artificial sequence
  synthetic sequence
SPDLSKVR

SEQ ID NO: 363 - protein, 8 aa, Artificial sequence
  synthetic sequence
SPGPQRER

SEQ ID NO: 364 - protein, 7 aa, Artificial sequence
  synthetic sequence
AQGMDYL

SEQ ID NO: 365 - protein, 7 aa, Artificial sequence
  synthetic sequence
DLLFVSK

SEQ ID NO: 366 - protein, 7 aa, Artificial sequence
  synthetic sequence
FLPNKQR

SEQ ID NO: 367 - protein, 7 aa, Artificial sequence
  synthetic sequence
HIEALLD

SEQ ID NO: 368 - protein, 7 aa, Artificial sequence
  synthetic sequence
HVNILLF

SEQ ID NO: 369 - protein, 7 aa, Artificial sequence
  synthetic sequence
IDIARQT

SEQ ID NO: 370 - protein, 7 aa, Artificial sequence
  synthetic sequence
LASIELL

SEQ ID NO: 371 - protein, 7 aa, Artificial sequence
  synthetic sequence
PDLSKVR

SEQ ID NO: 372 - protein, 7 aa, Artificial sequence
  synthetic sequence
RDQIIFM

SEQ ID NO: 373 - protein, 7 aa, Artificial sequence
  synthetic sequence
RPLFPQI

SEQ ID NO: 374 - protein, 7 aa, Artificial sequence
  synthetic sequence
SIELLAR

SEQ ID NO: 375 - protein, 7 aa, Artificial sequence
  synthetic sequence
SVLPSSL

SEQ ID NO: 376 - protein, 7 aa, Artificial sequence
  synthetic sequence
VFLPNKQ

SEQ ID NO: 377 - protein, 16 aa, Artificial sequence
  synthetic sequence
TALSGSSPSAPASDSI

SEQ ID NO: 378 - protein, 15 aa, Artificial sequence
  synthetic sequence
AALSGGGGGGAEPSP

SEQ ID NO: 379 - protein, 15 aa, Artificial sequence
  synthetic sequence
MAALSGGGAGAGAGA

SEQ ID NO: 380 - protein, 15 aa, Artificial sequence
  synthetic sequence
TGTPPASLPGSLTNV

SEQ ID NO: 381 - protein, 15 aa, Artificial sequence
  synthetic sequence
TTGPPASLPGSLTNV

SEQ ID NO: 382 - protein, 14 aa, Artificial sequence
  synthetic sequence
SAMDTVTSSSSSSL

SEQ ID NO: 383 - protein, 14 aa, Artificial sequence
  synthetic sequence
TALSGSSPSAPASD

SEQ ID NO: 384 - protein, 13 aa, Artificial sequence
  synthetic sequence
VTSSSSSVLPSSL

SEQ ID NO: 385 - protein, 12 aa, Artificial sequence
  synthetic sequence
ASVLPSSLSPQK

SEQ ID NO: 386 - protein, 12 aa, Artificial sequence
  synthetic sequence
GTVVTAPTPQQL

SEQ ID NO: 387 - protein, 12 aa, Artificial sequence
  synthetic sequence
LSATPPASPGSL

SEQ ID NO: 388 - protein, 12 aa, Artificial sequence
  synthetic sequence
SAPKTPIQAGGY

SEQ ID NO: 389 - protein, 12 aa, Artificial sequence
  synthetic sequence
SAVLPSSLSPQK

SEQ ID NO: 390 - protein, 12 aa, Artificial sequence
  synthetic sequence
SAVLSNPKSPQK

SEQ ID NO: 391 - protein, 12 aa, Artificial sequence
  synthetic sequence
TDISWLTGVEVL

SEQ ID NO: 392 - protein, 12 aa, Artificial sequence
  synthetic sequence
TVGQRGSFGTVY

SEQ ID NO: 393 - protein, 11 aa, Artificial sequence
  synthetic sequence
ALQKSPGPQER

SEQ ID NO: 394 - protein, 11 aa, Artificial sequence
  synthetic sequence
HVYSTKPQLAI

SEQ ID NO: 395 - protein, 11 aa, Artificial sequence
  synthetic sequence
IGPQILTSPSR

SEQ ID NO: 396 - protein, 11 aa, Artificial sequence
  synthetic sequence
LNVTAPTPQLQ

SEQ ID NO: 397 - protein, 11 aa, Artificial sequence
  synthetic sequence
NNTIEPVNIDD

SEQ ID NO: 398 - protein, 11 aa, Artificial sequence
  synthetic sequence
SVLPSSLSVPK

SEQ ID NO: 399 - protein, 10 aa, Artificial sequence
  synthetic sequence
HINTIEPVDL

SEQ ID NO: 400 - protein, 10 aa, Artificial sequence
  synthetic sequence
INNRDQIFMV

SEQ ID NO: 401 - protein, 10 aa, Artificial sequence
  synthetic sequence
IQDGEKKPIS

SEQ ID NO: 402 - protein, 9 aa, Artificial sequence
  synthetic sequence
DERPLFPQL

SEQ ID NO: 403 - protein, 9 aa, Artificial sequence
  synthetic sequence
FVRFFTLAF

SEQ ID NO: 404 - protein, 9 aa, Artificial sequence
  synthetic sequence
NVPLTTNFH

SEQ ID NO: 405 - protein, 9 aa, Artificial sequence
  synthetic sequence
RDEPLFPQI

SEQ ID NO: 406 - protein, 8 aa, Artificial sequence
  synthetic sequence
EKPVEVLE

SEQ ID NO: 407 - protein, 8 aa, Artificial sequence
  synthetic sequence
IGDFGLSR

SEQ ID NO: 408 - protein, 8 aa, Artificial sequence
  synthetic sequence
IKQIEALL

SEQ ID NO: 409 - protein, 8 aa, Artificial sequence
  synthetic sequence
KARGLIPE

SEQ ID NO: 410 - protein, 8 aa, Artificial sequence
  synthetic sequence
KLASIELL

SEQ ID NO: 411 - protein, 8 aa, Artificial sequence
  synthetic sequence
LSGLFPAI

SEQ ID NO: 412 - protein, 7 aa, Artificial sequence
  synthetic sequence
HQLLASL

SEQ ID NO: 413 - protein, 13 aa, Artificial sequence
  synthetic sequence
LEEKKGNYVVTDH

SEQ ID NO: 414 - protein, 13 aa, Artificial sequence
  synthetic sequence
LEEKKYNYVVTDH

SEQ ID NO: 415 - protein, 11 aa, Artificial sequence
  synthetic sequence
RSLAPEVRGYW

SEQ ID NO: 416 - protein, 11 aa, Artificial sequence
  synthetic sequence
WTSSPEIRAVF

SEQ ID NO: 417 - protein, 11 aa, Artificial sequence
  synthetic sequence
ASSRPELALAY

SEQ ID NO: 418 - protein, 11 aa, Artificial sequence
  synthetic sequence
WTSHPEIKAAF

SEQ ID NO: 419 - protein, 11 aa, Artificial sequence
  synthetic sequence
RSLKPEVREVF

SEQ ID NO: 420 - protein, 11 aa, Artificial sequence
  synthetic sequence
ASLRPEVREAF

SEQ ID NO: 421 - protein, 11 aa, Artificial sequence
  synthetic sequence
KSLYPEIREVF

SEQ ID NO: 422 - protein, 11 aa, Artificial sequence
  synthetic sequence
LSGVPEIRERW

SEQ ID NO: 423 - protein, 11 aa, Artificial sequence
  synthetic sequence
LTIRPEIRPRW

SEQ ID NO: 424 - protein, 11 aa, Artificial sequence
  synthetic sequence
ASFKPELPDFF

SEQ ID NO: 425 - protein, 11 aa, Artificial sequence
  synthetic sequence
STISPEIRLFW

SEQ ID NO: 426 - protein, 11 aa, Artificial sequence
  synthetic sequence
ASLKPEVPLVF

SEQ ID NO: 427 - protein, 11 aa, Artificial sequence
  synthetic sequence
SSGAPEVRELF

SEQ ID NO: 428 - protein, 11 aa, Artificial sequence
  synthetic sequence
SSVVPELPMAF

SEQ ID NO: 429 - protein, 11 aa, Artificial sequence
  synthetic sequence
RSFYPELRLLF

SEQ ID NO: 430 - protein, 11 aa, Artificial sequence
  synthetic sequence
LTISPEIPPYF

SEQ ID NO: 431 - protein, 11 aa, Artificial sequence
  synthetic sequence
ESFRPEIRQYF

SEQ ID NO: 432 - protein, 11 aa, Artificial sequence
  synthetic sequence
GSLSPELRPIF

SEQ ID NO: 433 - protein, 11 aa, Artificial sequence
  synthetic sequence
STLSPELRGRW

SEQ ID NO: 434 - protein, 11 aa, Artificial sequence
  synthetic sequence
KSFRPELKEFY

SEQ ID NO: 435 - protein, 11 aa, Artificial sequence
  synthetic sequence
KSLTPEVRGYW

SEQ ID NO: 436 - protein, 11 aa, Artificial sequence
  synthetic sequence
WTSHPEIRAYF

SEQ ID NO: 437 - protein, 11 aa, Artificial sequence
  synthetic sequence
ASFRPELALRY

SEQ ID NO: 438 - protein, 11 aa, Artificial sequence
  synthetic sequence
KSLTPEVREYF

SEQ ID NO: 439 - protein, 11 aa, Artificial sequence
  synthetic sequence
KSLAPEVRELF

SEQ ID NO: 440 - protein, 11 aa, Artificial sequence
  synthetic sequence
RSFYPEIREYF

SEQ ID NO: 441 - protein, 11 aa, Artificial sequence
  synthetic sequence
LSLRPEIREYW

SEQ ID NO: 442 - protein, 11 aa, Artificial sequence
  synthetic sequence
GTIRPEIREMW

SEQ ID NO: 443 - protein, 11 aa, Artificial sequence
  synthetic sequence
NSFKPEIPDYF

SEQ ID NO: 444 - protein, 11 aa, Artificial sequence
  synthetic sequence
GTISPEIREMW

SEQ ID NO: 445 - protein, 11 aa, Artificial sequence
  synthetic sequence
LSLRPEVPLFF

SEQ ID NO: 446 - protein, 11 aa, Artificial sequence
  synthetic sequence
SSGVPEVRMMF

SEQ ID NO: 447 - protein, 11 aa, Artificial sequence
  synthetic sequence
SSVVPEVRMMF

SEQ ID NO: 448 - protein, 11 aa, Artificial sequence
  synthetic sequence
GTIRPEIPDYF

SEQ ID NO: 449 - protein, 11 aa, Artificial sequence
  synthetic sequence
LSGSPELRMIF

SEQ ID NO: 450 - protein, 11 aa, Artificial sequence
  synthetic sequence
SSFSPELRMRW

SEQ ID NO: 451 - protein, 11 aa, Artificial sequence
  synthetic sequence
ASFRPELAEFW

SEQ ID NO: 452 - protein, 13 aa, Artificial sequence
  synthetic sequence
  VARIANT (6)..(6): The amino acid at position 6 is either Gly or Tyr
LEEKKXNYVVTDH

(19) United States
(12) Patent Application Publication
(10) Pub. No.: US 2024/0066115 A1
(43) Pub. Date: Feb. 29, 2024

(54) DETERMINATION AND USES OF CD8+ T CELL EPITOPES

(71) Applicant: The Board of Trustees of the Leland Stanford Junior University, Stanford, CA (US)

(72) Inventor: Albert J. Wong, Menlo Park, CA (US)

(21) Appl. No.: 18/271,420

(22) PCT Filed: Feb. 1, 2022
(86) PCT No.: PCT/US2022/014756

§ 371 Date: Jul. 7, 2023

Related U.S. Application Data
(60) Provisional application No. 63/144,250, filed on Feb. 1, 2021.

(51) Int. Cl. A61K 39/215; C07K 14/005; G01N 33/68; H01J 49/00; H01J 49/16

(52) U.S. Cl. CPC A61K 39/215; C07K 14/005; G01N 33/6848; H01J 49/004; H01J 49/164

(57) ABSTRACT

Compositions and methods are provided for the identification of peptide sequences that are presented to T cells in an MHC context.

### CROSS REFERENCE TO RELATED APPLICATION

The present application claims the benefit of and priority to U.S. Provisional Patent Application No. 63/144,250, filed Feb. 1, 2021, the entire disclosure of which is hereby.

### INCORPORATION BY REFERENCE OF SEQUENCE LISTING PROVIDED AS A TEXT FILE

A Sequence Listing is provided herewith in a text file, (S20-525_STAN-1824WO_SEQ_LIST_ST25.txt), created on Feb. 1, 2022, and having a size of 90,000 bytes. The contents of the text file are incorporated herein by reference in its entirety.

### BACKGROUND

T cells are the central mediators of adaptive immunity, through both direct effector functions and coordination and activation of other immune cells. Each T cell expresses a unique T cell receptor (TCR), selected for the ability to bind to major histocompatibility complex (MHC) molecules presenting peptides. TCR recognition of peptide-MHC (pMHC) drives T cell development, survival, and effector functions. Even though TCR ligands are relatively low affinity (1-100 μM), the TCRs are remarkably sensitive, requiring as few as 1 agonist peptide to activate a T cell.

Proteasomes are multi-subunit enzyme complexes in eukaryotic cells that selectively degrade endogenous proteins into oligopeptides. Their activity is important for protein quality control and for regulation of many intracellular processes including cell cycle progression, signaling pathways and transcription. A subset of oligopeptides generated by proteasomes are translocated from the cytoplasm into the endoplasmic reticulum (ER) by the transporter associated with antigen presentation (TAP), where they may associate with newly-synthesized human leukocyte antigen class I (HLA-1) molecules. Peptide-loaded HLA-I molecules then traffic to the cell surface for display to CD8⁺T cells, enabling immunosurveillance of tumors and cells infected by pathogens such as viruses or bacteria.

Peptide antigens were thought to be contiguous sequences originating from self- or foreign proteins. However, evidence has accumulated that proteasome-catalyzed peptide splicing (PCPS) reactions comprise an additional source of peptides that can be presented on HLA-I molecules for CD8⁺ T cell recognition. Primarily proteasomes generate peptides composed of fragments from within the same protein/polypeptide via cis-splicing. The constitutive proteasome (CP) and the immunoproteasome (IP) both mediate PCPS reactions, and the thymoproteasome has recently also been shown to do so. PCPS can be initiated at proteasomal active sites by catalytic threonine residues that perform nucleophilic attack on carbonyl groups within an unfolded polypeptide chain, and can occur in either a forward or reverse sense. This forms an acyl-enzyme intermediate tethered to the proteasome by an ester linkage. Nucleophilic attack of the acyl-enzyme intermediate by a free amine group liberated by proteasomal cleavage of a non-adjacent peptide fragment within the precursor substrate then hydrolyses the ester linkage, appending the C-terminal portion of the final spliced peptide product.

Knowledge of PCPS reactions and products allow for improved analysis and development of antigenic peptides; and the effect of such peptides on T cell recognition.

### SUMMARY OF THE INVENTION

Compositions and methods are provided for the identification of peptide sequences that are presented on Class I MHC proteins to CD8+ T cells, which peptides can be a co-linear, contiguous, sequence of a protein of interest, or can be derived from proteasome-catalyzed peptide splicing (PCPS) of the protein of interest. In some embodiments, specific peptides thus identified are provided for certain proteins of interest. Methods are also provided for the modification of peptide sequences to optimize proteasome digestion, and presentation to T cells. The methods and compositions described herein can be used to identify immunogenic antigen peptides, which peptides can be used as immunogens, to develop drugs, such as personalized medicine drugs, and isolation and characterization of antigen-specific T cells.

In some embodiments, a protein of interest for identifying immunogenic peptide epitopes is a cancer cell protein, e.g. a cancer neoantigen, a protein selectively expressed in cancer cells, a protein over-expressed in cancer cells, and the like. In some embodiments, a protein of interest for identifying immunogenic peptide epitopes is a pathogen protein, e.g. virus, bacteria, parasite, etc. proteins. In some embodiments, peptide sequence epitopes are identified. In some embodiments, peptide sequence epitopes are selected for use, e.g. in diagnostic and therapeutic methods. In some embodiments, a therapeutic method is preparation of peptides for cancer therapy. In other embodiments, a therapeutic method is preparation of a vaccine to a pathogen.

The sequence of peptide antigens related to the 13 aa pepvIII vaccine are provided in Tables 1A-1B, which peptides are co-linear or PCPS fragments. These peptides resulted from proteasomal cleavage; and have been shown to bind to both MHC-I and HLA-1. Any of the peptides, or a cocktail of peptides, set forth in SEQ ID NO:1-10 and 11-75 are provided, and can be used in tumor vaccination schemes for EGFRvIII positive cancers. The peptides can also be used in diagnostic schemes or patient monitoring using patient PBMCs.

Peptide antigens related to SARS-CoV2 spike protein are provided in Tables 2A-2B SEQ ID NO:76-117 and SEQ ID NO:118-253, respectively, and are co-linear or resulting from PCPS, as marked. Peptides from the SARS-CoV2 nucleocapsid protein are provided in Table 3, SEQ ID NO:254-308, which are co-linear or PCPS fragments. These peptides resulted from proteasomal cleavage. Any of the peptides, or a cocktail of peptides, set forth in Tables 2A, 2B and 3 are provided, and can be used in virus vaccination schemes for SARS-CoV2. They can also be used in diagnostic schemes or patient monitoring using patient PBMCs.

Peptide antigens related to human B-raf protein comprising a V600E mutation are provided in Tables 4A (SEQ ID NO:309-376) and 4B (SEQ ID NO:377-412), and are co-linear or resulting from PCPS, as marked. These peptides resulted from proteasomal cleavage. Any of the peptides, or a cocktail of peptides, set forth in Tables 2A, 2B and 3 are provided, and can be used in cancer vaccination schemes for B-raf associated cancers. They can also be used in diagnostic schemes or patient monitoring using patient PBMCs.

In some embodiments, a method is provided for the rapid identification of CD8⁺ T cell epitopes. The methods comprise incubation of a candidate protein of interest with activated 20S immunoproteasome and a molar excess of PA28 activator alpha subunit protein for a period of time sufficient to digest the protein, e.g. from about 12 to about 36 hours, and may be around 24 hours. Candidate proteins of greater than about 50 amino acids may be pre-treated by denaturation, e.g. by incubation in urea, prior to digestion with the immunoproteasome. The proteasome digest is then immunoprecipitated with Class I MHC molecules, e.g. human HLA-A, B, C Class I molecules, for example by incubation with a substrate comprising immobilized HLA proteins, followed by washing the substrate free of unbound peptides. The bound peptides can then be eluted and analyzed for molecular weight, sequencing, mass spectrometric methods such as MALDI-ToF or LC-MS/MS, and/or de novo sequencing by mass spectrometry or chemical sequencing such as by Edman degradation.

In de novo sequencing of the eluted peptides, a computer algorithm matches the molecular weight, for example as determined by mass spectrometry, with the same molecular weights from the known sequences in a database. A peptide can be matched across the b- and y-series of fragments to a known sequence. While co-linear (or contiguous) peptides are efficiently identified by this method, such matches are more difficult for spliced (PCPS) peptides. Provided herein are methods for generating a database useful in matching spliced peptides.

In one embodiment to generate a database for a peptide-spectra match (PSM) search, the molecular weights observed through MALDI-TOF are aggregated. Taking the range of observed original molecular weights, the potential linear and PCPS-derived rearrangements of the parental sequence that match the original weight molecular weight are calculated, where the peptides are restricted to lengths between about 8 and about 12 amino acid residues in length. This algorithm is used to generate a FASTA database of co-linear and PCPS spliced peptides that are used for de novo sequencing by MS/MS. In some embodiments, this method is applied to peptides of less than about 100 aa in length, or less than about 50 aa in length.

In other embodiments involving larger proteins, e.g. greater than about 50 aa in length, greater than about 100 aa in length, or more, molecular weight data from MALDI-ToF is used to generate a database of the co-linear fragments from a proteasome digestion. These co-linear fragments are directly derived from the parental protein. From this data, the fragments from 2 to 12 aa are used to construct a database that encompasses possible PCPS recombinants. A boundary may be set on the distance of cis-ligation, e.g. more than 1 aa, less than about 50 aa, less than about 30 aa distance, between the sites of splicing. Boundaries on the peptide size in the database may be set at from about 8 to about 12 amino acids in length. An algorithm is then used to assemble a database where 2-10 aa fragments across any given 50 aa window are used in combinatorial fashion to make hypothetical PCPS sequences of between 8-12 aa, containing no more than 3 fragments.

In another embodiment, a database for peptide identification is developed without using experimental identification of fragments. All possible fragments ranging from 2 to 10 aa's are identified within a candidate protein sequence. The 2-10 aa fragments across any given 50 aa window are used in combinatorial fashion to make hypothetical PCPS sequences of between 8-12 aa but containing no more than 3 fragments.

In some embodiments a universal database is developed where a protein sequence can be plugged in generate the actual fragments. In this embodiment, all possible windows of from 2 to 12 amino acids across a sequence of about 50 to about 70 amino acids are generated. This list of sequences is used to generate all possible recombinants of from 1 to 3 fragments that range from 8-12 aa. Redundant sequences are pruned. To continue walking across the entire protein sequence, peptide windows containing the first amino acid are eliminated and windows including the next amino acid in the sequence are added. Because the vast majority of recombinations will be the same, it is only necessary to contemplate the recombinations arising from the new amino acid.

Once co-linear and PCPS fragments that bind to HLA-I are identified through a matching algorithm to a database as disclosed above, the peptides are optionally confirmed for binding to an appropriate class I MHC in an MHC stabilization assay. Such assays include, without limitation, incubation of a candidate peptide with a defective transporter associated with antigen processing (TAP) cell lines, such as T2 (human) or RMA-S (mouse) and a cell expressed the targeted MHC class I protein, where the presence of a stabilized MHC protein/peptide complex can be detected by any convenient method. Peptides can also be subjected to functional assays, e.g. determining induction of a T cell response; binding to CD8+ T cells, and the like as appropriate for the peptide.

In other embodiments, a method is provided for enhancing proteasomal cleavage of a polypeptide antigen by sequence modification, in order to increase production of co-linear and PCPS fragments. The increase in production of fragments will enhance the immunologic response against the original protein sequence. In such embodiments, amino acid residues that create a hairpin in the structure of a protein antigen are modified to remove or replace the residue. In one example, the glycine present at residue 6 of the EGFRvIII tumor vaccine (pepvIII) (SEQ ID 413: LEEKKGNYVVTDH), is replaced with tyrosine to enhance presentation of the antigen to T cells. In some embodiments, a peptide of sequence (SEQ ID NO:414) LEEKKYNYVVTDH is provided, which peptide finds use as an antigen for anti-tumor vaccination.

Also provided herein are software products tangibly embodied in a machine-readable medium, the software product comprising instructions operable to cause one or more data processing apparatus to perform operations comprising the methods of database generation for sequence matching described herein. A database of sequences is searched with the disclosed algorithms to identify co-linear and PCPS fragments generated by proteasomal digestion of a candidate polypeptide.

### BRIEF DESCRIPTION OF THE DRAWINGS

FIG. 1A-G: Substitutions at the GLY-6 position of pepvIII may alter its activity. A) Model of the predicted tertiary structure of pepvIII as predicted by Jmol software (Gly-6 highlighted in red) (PDB+Jmol/JSmol Web Portal). B) Diagram of vaccination schedule for EGFRvIII+HC2 20d2/c subcutaneous tumor model. C) Impact of vaccination with substituted peptide variants on EGFRvIII+HC2 20d2/c subcutaneous tumor model. D) Comparison of KLH treated control (green), pepvIII vaccinated (red) and Y6-pepvIII vaccinated (blue) in the EGFRvIII+HC2 20d2/c subcutaneous tumor model (Log-rank; n=11 KLH, 21 pepvIII and 9 Y6-pepvIII). E) Tumor volume in EGFRvIII+HC2 20d2/c subcutaneous tumor model (Two-way ANOVA; n=6 KLH, 7 pepvIII, and 6 Y6-pepvIII). F) Model of the predicted tertiary structure of pepvIII and Y6-pepvIII (Tyr-6 highlighted in blue) as predicted by PEP-FOLD and Jmol software. G) Predicted proteasomal cleavage sites in pepvIII (left panel) and the Y6-pepvIII (right panel) as predicted by NET-CHOP software. S denotes predicted cleavage sites, the score value indicates how likely cleavage at the denoted site is to occur. Bars represent mean±S.D. *** denotes p<0.001, ****p<0.0001.

FIG. 2A-E: Efficacy of the TYR-6 variant peptide in an intracranial GBM model. A) Diagram of the vaccination schedule for GL261/vIII intracranial tumor model. B) Overall survival of GL261/vIII tumor bearing mice after vaccination (Log-rank; n=10 Montanide control, 14 Montanide+KLH, 10 Montanide+pepvIII, and 27 Montanide+Y6-pepvIII). C) Cytotoxic T cell killing assay assessing cytolytic capacity of T cells from KLH, pepvIII, and Y6-pepvIII vaccinated mice against GL261/vIII (left-panel) and GL261/wt (right-panel) target cells (Two-way ANOVA; n=3 independent experiments). D) ELIspot assay to assess the cytokine production capacity of bulk splenic T cells derived from pepvIII and Y6-pepvIII vaccinated mice (One-way ANOVA, n=4 mice in 2 independent experiments). E) Effect of vaccination in CD4 and CD8 T cell depleted GL261/vIII tumor bearing mice (Log-rank; n=10 KLH+Montanide, 11 Y6-pepvIII+Montanide, 5 CD4+ T cell depleted, and 5 CD8+ T cell depleted). Bars represent mean±S.D. * denotes p<0.05, **p<0.01, ***p<0.001, ****p<0.0001.

FIG. 3A-F: The effect of Y6-pepvIII vaccination on intratumoral lymphocyte populations A) Proportion of tumor infiltrating CD4+ T cells represented as % of total intratumoral CD45+ leukocytes (Mann-Whitney; n=9 KLH, 7 pepvIII and 7 Y6-pepvIII). B) Proportion of tumor infiltrating CD4+/CD25+ T cells represented as % of total intratumoral CD4+ T cells (Mann-Whitney; n=9 KLH, 7 pepvIII and 7 Y6-pepvIII). C) Proportion of tumor infiltrating CD8+ T cells represented as % of total intratumoral CD45+ leukocytes (Mann-Whitney; n=9 KLH, 7 pepvIII and 8 Y6-pepvIII). D) Representation of intratumoral CD8 to CD4 ratio in GL261/vIII tumor bearing mice (Mann-Whitney; n=10 KLH, 10 pepvIII and 10 Y6-pepvIII). E) Proportion of tumor infiltrating CD11c+ cells as represented as % of total intratumoral CD45+ cells (Mann-Whitney; n=9 KLH, 7 pepvIII and 7 Y6-pepvIII). F) Proportion of tumor infiltrating CD161+(NK1.1) cells represented as a % of total intratumoral CD45+ cells (Mann-Whitney; n=9 KLH, 7 pepvIII and 7 Y6-pepvIII). Bars represent mean±S.D. * denotes p<0.05, **p<0.01, ***p<0.001, ****p<0.0001.

FIG. 4A-E: Anti-PD-1 therapy potentiates Y6-pepvIII vaccine efficacy. A) Expression of PD-1 on the surface of intratumoral CD4+ T cells (Mann-Whitney; n=10 KLH, 8 pepvIII, and 10 Y6-pepvIII). B) Expression of PD-1 on the surface of intratumoral CD8+ T cells (Mann-Whitney; n=10 KLH, 8 pepvIII, and 10 Y6-pepvIII). C) Histogram demonstrating the difference in PD-1 expression on intratumoral CD8+ T cells across vaccination cohorts. D) Diagram of the vaccination schedule for single and combinational therapy in the GL261/vIII intracranial tumor model. E) Disease-free survival of GL261/vIII intracranial tumor bearing mice after treatment with anti-PD1 antibody alone or anti-PD1 in combination with Y6-pepvIII (Log-rank; n=10 KLH, 11 Y6-pepvIII, 6 anti-PD-1 only, and 11 Y6-pepvIII+anti-PD1). Bars represent mean±S.D. * denotes p<0.05, **p<0.01, ***p<0.001, ****p<0.0001.

FIG. 5A-E: Differences in the proteasomal processing of pepvIII and Y6-pepvIII may underlie enhanced vaccine efficacy A) T2 cell assay comparing the HLA-A2 stabilization of the parental pepvIII and Y6-pepvIII peptides in comparison to 3 control peptides identified by Wu et al (38) and Influenza M1 peptide as a positive control (ANOVA; n=at least 2 independent replicates. B) Representative averaged MALDI-TOF spectra of pepvIII (left panel) and Y6-pepvIII (right panel) incubated with activated 20s immunoproteasome for 30 minutes. m/z values are indicated for major peaks and represent the average of 3 independent runs. C) Time course analysis of digestion of Y6-pepvIII and pepvIII by immunoproteasome. The percentage of parental peptide remaining at various time periods is shown as mean±S.D with p<0.0001 for all time points. D) Summary of origin of peptides derived from 20s immunoproteasome digestion of pepvIII and Y6-pepvIII as determined by MALDI-TOF spectral analysis. The difference in size between glycine and tyrosine was considered in determining the fragments in common. E) Overlay of MALDI-TOF spectra depicting repertoire of peptide fragments eluted from HLA immunoprecipitation of peptide products produced from 2 hr. immunoproteasome digestion of pepvIII (red), Y6-pepvIII (green), or background control (yellow). Red box highlights proteasome derived HLA-binding products ranging from −875-1050 Da (estimated weight of typical 8-10mers). Each spectrum represents average of 8-10 individual spectra.

FIG. 6A-E: LC-MS/MS analysis of peptides produced by proteasomal processing of parental peptides A) Distribution and utilization frequency of proteasomal cleavage sites across pepvIII and Y6-pepvIII parental peptides (SEQ ID NO:452). B) Table outlining amino acid sequence of PCPS recombination products uniquely derived from proteasomal processing of Y6-pepvIII (from top to bottom SEQ ID NOs:3, 9, 1, 2, 4, 8, 7, 5, 6, 10). C) Heat map characterization of total putative LC-MS/MS peptide library by Hamming distance vs. novel amino acid contacts (left panel). Position of confirmed Y6-pepvIII derived PCPS products denoted by white circles and summarized in accompanying table (from top to bottom SEQ ID NOs:8, 7, 5, 6, 3, 9, 1, 2, 4). D) Characterization of HLA/MHC stabilization capacity of Y6-pepvIII derived PCPS products (left panel=HLA-A2, middle panel=HLA-B7, right panel=H-2Kb), stabilization capacity of pepvIII and Y6-pepvIII parental peptides and all 10 identified antigenic products (from left to right for the x-axis of each plot SEQ ID NOs:1-10) was normalized to an isotype negative control. Positive controls for each MHC molecule are shown for reference (HLA-A2=Influenza M1, HLA-B7=EBNA (Epstein-Barr) peptide 1, H-2Kb=SV (Sendai Virus) 324-223). E) Overall survival of GL261/vIII tumor bearing mice after vaccination with candidate peptides (from top to bottom SEQ ID NOs:10, 7, 9, 6) identified by LC-MS/MS (n=8 mice per group), * denotes p<0.05, **p<0.01, ***p<0.001.

FIG. 7: Similar humoral responses derived from pepvIII and Y6-pepvIII vaccination. Humoral immune responses directed against EGFRvIII were measured by ELISA using serum derived from pepvIII and Y6 pepvIII vaccinated mice.

FIG. 8A-C: Mice vaccinated with Y6-pepvIII produce antibodies specific to EGFRvIII. The humoral immune response induced by peptide vaccination was measured by incubating an EGFRvIII negative cell line (NIH-3T3—Panel A), and EGFRvIII expressing cell line (HC2 20d2/c—Panel B) and a cell line that is EGFRvIII negative but overexpresses wild-type EGFR (CO12—Panel C) with serum derived from pepvIII or Y6-pepvIII vaccinated mice for 2 hrs. Primary antibody recognition of EGFRvIII was evaluated by flow cytometry using a PE tagged anti-mouse IgG secondary antibody. Serum antibody binding to EGFRvIII on the cell surface is indicated as fold change in MFI of the anti-mouse IgG secondary antibody normalized to serum from KLH controls.

FIG. 9: Longitudinal imaging of GL261/vIII tumor bearing mice vaccinated with Y6-pepvIII. Bioluminescent imaging of Luc+GL261/vIII tumors in control (left panels) and Y6-pepvIII vaccinated (right panels) mice at day 7, day 14 and day 21 post tumor cell implantation.

FIG. 10: Spot counts from ELISpot comparing splenic T cells from pepvIII and Y6-pepvIII vaccinated mice. T cells were isolated from the spleens of mice vaccinated three times with KLH alone, pepvIII or Y6-pepvIII. Detection and enumeration of IFN-γ producing T cells was quantified using ELISpot assay.

FIG. 11: Survival of GL261/vIII tumor bearing mice treated with anti-PD-1 alone or in combination with pepvIII vaccination. Overall survival of GL261/vIII tumor bearing mice after treatment with anti-PD-1 monoclonal antibody alone or in combination with pepvIII vaccination (Log-rank; n=10 Control, 6 anti-PD-1 alone, 10 pepvIII alone, and 6 pepvIII+anti-PD1, ns=no significant difference).

FIG. 12A-E: The effect of combinational treatments on intratumoral lymphocyte populations in GL261/vIII tumor bearing mice S6) A) Proportion of tumor infiltrating CD4+ T cells represented as % of total intratumoral CD45+ leukocytes B) Proportion of tumor infiltrating CD4+/CD25+ T cells represented as % of total intratumoral CD4+ T cells. C) Proportion of tumor infiltrating CD8+ T cells represented as % of total intratumoral CD45+ leukocytes. D) Proportion of tumor infiltrating CD11c+ cells as represented as % of total intratumoral CD45+ cells. F) Proportion of tumor infiltrating CD161+(NK1.1) cells represented as a % of total intratumoral CD45+ cells. Mann-Whitney; n=9 control, 5 anti-PD-1, 5 anti-PD-1+pepvIII, and 5 anti-PD1+Y6-pepvIII. Bars represent mean±S.D. * denotes p<0.05, **p<0.01, ***p<0.001, ****p<0.0001.

FIG. 13. Survival of CT2A/vIII tumor bearing mice vaccinated with pepvIII, Y6-pepvIII or combinational therapy with Y6-pepVIII and anti-PD1. Disease-free survival of CT2A/vIII intracranial tumor bearing mice after treatment with pepvIII, Y6-pepvIII, or Y6-pepvIII in combination with anti-PD-1 antibody (Log-rank; n=6 KLH, 6 pepvIII, 6 Y6-pepvIII, and 9 Y6-pepvIII+anti-PD1).] Bars represent mean±S.D. * denotes p<0.05, **p<0.01, ***p<0.001, ****p<0.0001.

FIG. 14: Proteasomal cleavage site distribution in LC-MS/MS search library (SEQ ID NO:452). Proteasomal cleavage site utilization and distribution across amino acid position for the theoretical search library used for LC-MS/MS analysis of peptide products. Peptides were included in the search library contingent on mass correspondence to peaks observed from MALDI-TOF. Comparison of the observed cute site utilization in identified candidate peptides (FIG. 6A) to the cut site distribution of LC-MS/MS search library (above) confirms FIG. 15: Ion fragmentation of candidate PCPS products identified by LC-MS/MS) Existence and sequence of candidate PCPS products derived from IP experiments were validated by overlaying the actual (LC-MS/MS derived) and theoretical ion series for each candidate sequence. Using the Organic Mass Spectrometry: OrgMassSpecR package in R (b- and y-series ions in peptide fragmentation mass spectrometry were calculated from the matched sequence and aligned with observed peaks for visual confirmation).

FIG. 15A-I: Ion fragmentation of candidate PCPS products (FIG. 15A-I include SEQ ID NOs:3, 9, 1, 2, 4, 8, 7, 5, 6, respectively) identified by LC-MS/MS

Table 5: Amino acid substitutions change overall proteasomal processing potential of pepvIII. Table illustrates how various amino acid substitutions at position 6 of the original pepvIII site affects the human 20s proteasomal cleavage score of both the substituted site and distal peptide bonds within the parental peptide as determined by NetChop software. Absolute scores and changes relative to the pepvIII parental sequence for each position are listed in the table. The “composite cleavage score” is a metric that demonstrated how amino acid substitution at position 6 changes the overall 20s proteasomal cleavage score of the total peptide as compared to the original pepvIII sequence.

Table 6: 1-hour digestion of substituted peptides with human 20s immunoproteasome. Table illustrates the percentage of parental peptide remaining after 1 hr. digestion of substituted peptides with the human 20s immunoproteasome. Values were calculated based on analysis of MALDI-TOF spectra (intensity of summed parental derivative peaks/intensity of summed non-parental peaks).

### DETAILED DESCRIPTION OF THE EMBODIMENTS

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, illustrative methods, devices and materials are now described.

All publications mentioned herein are incorporated herein by reference for the purpose of describing and disclosing the subject components of the invention that are described in the publications, which components might be used in connection with the presently described invention.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

MHC Proteins. Major histocompatibility complex proteins (also called human leukocyte antigens, HLA, or the H2 locus in the mouse) are protein molecules expressed on the surface of cells that confer a unique antigenic identity to these cells. MHC/HLA antigens are target molecules that are recognized by T-cells and natural killer (NK) cells as being derived from the same source of hematopoietic reconstituting stem cells as the immune effector cells (“self”) or as being derived from another source of hematopoietic reconstituting cells (“non-self”). Two main classes of HLA antigens are recognized: HLA class I and HLA class 1l.

The MHC proteins may be from any mammalian or avian species, e.g. primate sp., particularly humans; rodents, including mice, rats and hamsters; rabbits; equines, bovines, canines, felines; etc. Of particular interest are the class I human HLA proteins, and the murine H-2 proteins. Included in the HLA proteins are the class I proteins HLA-A, HLA-B, HLA-C, and β₂-microglobulin. Included in the murine H-2 subunits are the class I H-2K, H-2D, H-2L, and the class III-Aα, I-Aβ, I-Eα and I-Eβ, and β₂-microglobulin.

For experimental purposes the MHC binding domains may be a soluble form of the normally membrane-bound protein. The soluble form can be derived from the native form by deletion of the transmembrane domain. Conveniently, the protein is truncated, removing both the cytoplasmic and transmembrane domains.

An “allele” is one of the different nucleic acid sequences of a gene at a particular locus on a chromosome. One or more genetic differences can constitute an allele. An important aspect of the HLA gene system is its polymorphism. Each gene, MHC class I (A, B and C) and MHC class II (DP, DQ and DR) exists in different alleles. Current nomenclature for HLA alleles are designated by numbers, as described by Marsh et al.: Nomenclature for factors of the HLA system, 2010. Tissue Antigens 75:291-455, herein specifically incorporated by reference. For HLA protein and nucleic acid sequences, see Robinson et al. (2011), The IMGT/HLA database. Nucleic Acids Research 39 Suppl 1:D1171-6, herein specifically incorporated by reference.

MHC context. The function of MHC molecules is to bind peptide fragments derived from pathogens and display them on the cell surface for recognition by the appropriate T cells. Thus T cell receptor recognition can be influenced by the MHC protein that is presenting the antigen. The term MHC context refers to the recognition by a TCR of a given peptide, when it is presented by a specific MHC protein.

T cell receptor, refers to the antigen/MHC binding heterodimeric protein product of a vertebrate, e.g. mammalian, TCR gene complex, including the human TCR α, β, γ and δ chains. For example, the complete sequence of the human β TCR locus has been sequenced, as published by Rowen et al. (1996) Science 272(5269):1755-1762; the human α TCR locus has been sequenced and resequenced, for example see Mackelprang et al. (2006) Hum Genet. 119(3):255-66; see a general analysis of the T-cell receptor variable gene segment families in Arden Immunogenetics. 1995; 42(6):455-500; each of which is herein specifically incorporated by reference for the sequence information provided and referenced in the publication.

Peptide ligands of the TCR are peptide antigens against which an immune response involving T lymphocyte antigen specific response can be generated. Such antigens include antigens associated with autoimmune disease, infection, foodstuffs such as gluten, etc., allergy or tissue transplant rejection. Antigens also include various microbial antigens, e.g. as found in infection, in vaccination, etc., including but not limited to antigens derived from virus, bacteria, fungi, protozoans, parasites and tumor cells. Tumor antigens include tumor specific antigens, e.g. immunoglobulin idiotypes and T cell antigen receptors; oncogenes, such as B-raf, particularly comprising a V600E mutation, p21/ras, p53, p210/bcr-abl fusion product; etc.; developmental antigens, e.g. MART-1/Melan A; MAGE-1, MAGE-3; GAGE family; telomerase; etc.; viral antigens, e.g. human papilloma virus, Epstein Barr virus, etc.; tissue specific self-antigens, e.g. tyrosinase; gp100; prostatic acid phosphatase, prostate specific antigen, prostate specific membrane antigen; thyroglobulin, α-fetoprotein; etc.; and self-antigens, e.g. her-2/neu; carcinoembryonic antigen, muc-1, EGFRvIII and the like.

“Suitable conditions” shall have a meaning dependent on the context in which this term is used. That is, when used in connection with binding of a T cell receptor to a polypeptide epitope, the term shall mean conditions that permit a TCR to bind to a cognate peptide ligand. When this term is used in connection with nucleic acid hybridization, the term shall mean conditions that permit a nucleic acid of at least 15 nucleotides in length to hybridize to a nucleic acid having a sequence complementary thereto. When used in connection with contacting an agent to a cell, this term shall mean conditions that permit an agent capable of doing so to enter a cell and perform its intended function. In one embodiment, the term “suitable conditions” as used herein means physiological conditions.

The term “specificity” refers to the proportion of negative test results that are true negative test result. Negative test results include false positives and true negative test results.

The term “sensitivity” is meant to refer to the ability of an analytical method to detect small amounts of analyte. Thus, as used here, a more sensitive method for the detection of amplified DNA, for example, would be better able to detect small amounts of such DNA than would a less sensitive method. “Sensitivity” refers to the proportion of expected results that have a positive test result.

The term “reproducibility” as used herein refers to the general ability of an analytical procedure to give the same result when carried out repeatedly on aliquots of the same sample.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “sequence identity,” as used herein in reference to polypeptide or DNA sequences, refers to the subunit sequence identity between two molecules. When a subunit position in both of the molecules is occupied by the same monomeric subunit (e.g., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent.

By “parent polypeptide”, “protein of interest”, “parent protein”, “precursor polypeptide”, or “precursor protein” as used herein is meant an unmodified polypeptide that is subsequently modified to generate a variant or from which peptide fragments are obtained. A parent polypeptide may be a wild-type (or native) polypeptide, or a variant or engineered version of a wild-type polypeptide. Parent polypeptide may refer to the polypeptide itself, compositions that comprise the parent polypeptide, or the amino acid sequence that encodes it.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. “Amino acid analogs” refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acid modifications disclosed herein may include amino acid substitutions, deletions and insertions, particularly amino acid substitutions. Variant proteins may also include conservative modifications and substitutions at other positions of the cytokine and/or receptor (e.g., positions other than those involved in the affinity engineering). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: Ala, Pro, Gly, Gln, Asn, Ser, Thr; Group II: Cys, Ser, Tyr, Thr; Group Ill: Val, lie, Leu, Met, Ala, Phe; Group IV: Lys, Arg, His; Group V: Phe, Tyr, Trp, His; and Group VI: Asp, Glu. Further, amino acid substitutions with a designated amino acid may be replaced with a conservative change.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of cancer in a mammal, particularly in a human, and includes: (a) inhibiting the disease, i.e., arresting its development; and (b) relieving the disease or its symptoms, i.e., causing regression of the disease or its symptoms.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of engineered cells to prevent or delay, to alleviate, or to arrest or inhibit development of the symptoms or conditions associated with disease or other diseases. The term “therapeutic effect” refers to the reduction, elimination, or prevention of the disease, symptoms of the disease, or side effects of the disease in the subject.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease, e.g., to delay or minimize the growth and spread of cancer. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses, e.g. vaccine doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

“In combination with”, “combination therapy” and “combination products” refer, in certain embodiments, to the concurrent administration to a patient of the engineered proteins and cells described herein in combination with additional therapies, e.g. surgery, radiation, chemotherapy, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

### Therapeutic Methods

Immunotherapy using tumor-specific peptides has been described, for example with the EGFRvIII tumor vaccine (pepvIII). In such methods, an immunogen derived from a tumor protein of interest is administered to a cancer patient in a dose sufficient to generate an immune response, e.g. a CD8+ immune response, to cancer cells expressing the tumor protein of interest. Tumor proteins include, for example, neoantigens, over-expressed antigens, selectively expressed antigens, and the like.

Tumor neoantigens may arise as a result of genetic change (e.g., inversions, translocations, deletions, missense mutations, splice site mutations, etc.) within malignant cells, represent the most tumor-specific class of antigens. Mutations in B-raf, which are associated with a number of cancers including, without limitation, colorectal cancer, are an example of neoantigens. The serine/threonine protein kinase BRAF is an important player in the epidermal growth factor receptor (EGFR)-mediated mitogen-activated protein kinase (MAPK) pathway, where it is activated by the RAS small GTPase. The strength of BRAF is to not only activate the MAPK pathway that profoundly affects cell growth, proliferation, and differentiation but also affect other key cellular processes, such as cell migration (through RHO small GTPases), apoptosis (through the regulation of BCL-2), and survival (through the HIPPO pathway). BRAF is found constitutively activated by mutation in 15% of all human known cancer types. BRAF was reported to be mutated at several sites; however, the vast majority of mutated BRAF are V600E (1799T>A nucleotide change), characterizing up to 80% of all BRAF mutations. This mutation results in amino acid change that confers constitutive kinase activity.

Various tumor antigens are known in the art. Efficiently choosing which particular peptides of an antigen to utilize as an immunogen requires the ability to predict which tumor-specific peptides will efficiently bind to the HLA alleles present in a patient and would be effectively presented to the patient's immune system for inducing anti-tumor immunity. One of the critical barriers to developing curative and tumor-specific immunotherapy is the identification and selection of highly specific and restricted tumor antigens to avoid autoimmunity. In some embodiments, a peptide for cancer vaccination is a peptide set forth in Table 1 or Table 4, including PCPS peptide set forth in one of Table 1A, Table 1B, or Table 4B.

For example, translating peptide sequencing information into a therapeutic vaccine can include prediction of peptides that can bind to HLA peptides of a high proportion of individuals; and may include optimizing those peptides for efficient presentation. Synthetic peptides provide a useful means to prepare multiple immunogens efficiently and to rapidly translate identification of epitopes to an effective vaccine. Peptides can be readily synthesized chemically and easily purified utilizing reagents free of contaminating bacteria or animal substances. The small size allows a clear focus on the mutated region of the protein and also reduces irrelevant antigenic competition from other components (unmutated protein or viral vector antigens).

Translating peptide sequencing information into a therapeutic vaccine can include a combination with a strong vaccine adjuvant. Effective vaccines can require a strong adjuvant to initiate an immune response. For example, poly-ICLC, an agonist of TLR3 and the RNA helicase-domains of MDA5 and RIG3, has shown several desirable properties for a vaccine adjuvant. These properties include the induction of local and systemic activation of immune cells in vivo, production of stimulatory chemokines and cytokines, and stimulation of antigen-presentation by DCs.

In some embodiments, immunogenic peptides are identified from cells from a subject with a disease or condition, and optionally modified to enhance presentation. In some embodiments, immunogenic peptides are specific to a subject with a disease or condition. In some embodiments, immunogenic peptides bind to an HLA that is matched to an HLA haplotype of a subject with a disease or condition.

In some embodiments, a library of peptides are expressed in the cells. In some embodiments, the cells comprise the peptides to be identified or characterized. In some embodiments, the peptides to be identified or characterized are endogenous peptides. In some embodiments, the peptides are exogenous peptides. For example, the peptides to be identified or characterized can be expressed from a plurality of sequences encoding a library of peptides.

Provided herein are methods of prediction of peptides, and optimization of peptides that to be presented by HLA class I proteins. In some embodiments, the application provides methods of identifying from a given set of antigen comprising peptides the most suitable peptides for preparing an immunogenic composition for a subject, said method comprising selecting from a given set of peptides the plurality of peptides processed by the immunoproteasome, determined by analyzing the sequence of peptides against peptide sequence databases as described herein. Examples of peptides are set forth in Tables 1, 2, 3 and 4.

Provided herein is a method of providing an anti-tumor immunity in a mammal comprising administering to the mammal a polynucleotide comprising a sequence encoding a peptide identified according to a method described, e.g. in Table 1A, Table 1B, Table 4A, Table 4B. Provided herein is a method of providing an anti-tumor immunity in a mammal comprising administering to the mammal an effective amount of a peptide with a sequence of a peptide identified according to a method described herein. Provided herein is a method of providing an anti-tumor immunity in a mammal comprising administering to the mammal a cell comprising a peptide comprising the sequence of a peptide identified according to a method described herein. Provided herein is a method of providing an anti-tumor immunity in a mammal comprising administering to the mammal a cell comprising a polynucleotide comprising a sequence encoding a peptide comprising the sequence of peptide identified according to a method described herein. In some embodiments, the cell presents the peptide as an HLA-peptide complex.

Provided herein is a method of treating a disease or disorder in a subject, the method comprising administering to the subject a polynucleotide comprising a sequence encoding a peptide identified according to a method described herein. Provided herein is a method of treating a disease or disorder in a subject, the method comprising administering to the subject an effective amount of a peptide comprising the sequence of a peptide identified according to a method described herein. Provided herein is a method of treating a disease or disorder in a subject, the method comprising administering to the subject a cell comprising a peptide comprising the sequence of a peptide identified according to a method described herein. Provided herein is a method of treating a disease or disorder in a subject, the method comprising administering to the subject a cell comprising a polynucleotide comprising a sequence encoding a peptide comprising the sequence of a peptide identified according to a method described herein. In some embodiments, the disease or disorder is cancer. In some embodiments the disease or disorder is an infection.

In some embodiments, the method comprises introducing one or more peptides to the population of cells. In some embodiments, the method comprises contacting the population of cells with the one or more peptides or expressing the one or more peptides in the population of cells. In some embodiments, the method comprises contacting the population of cells with one or more nucleic acids encoding the one or more peptides.

In some embodiments, the method comprises expressing a library of peptides in the population of cells. In some embodiments, the method comprises expressing a library of affinity acceptor tagged HLA-peptide complexes. In some embodiments, the library comprises a library of peptides associated with the disease or condition. In some embodiments, the disease or condition is cancer or an infection with an infectious agent or an autoimmune disease. In some embodiments, the method comprises characterizing one or more peptides from the HLA-peptide complexes specific an HLA class I protein of interest, optionally wherein the peptides are from one or more proteins of a pathogen or an autoantigen. In some embodiments, the method comprises characterizing one or more regions of the peptides from the one or more target proteins of the infectious agent or autoimmune disease.

In some embodiments, the infectious agent is a pathogen. In some embodiments, the pathogen is a virus, bacteria, or a parasite. In some embodiments, the virus is selected from the group consisting of: coronavirus, e.g. SARS-CoV1, SARS-CoV2, MERS, etc.; Dengue viruses (DENV-1, DENV-2, DENV-3, DENV-4, DENV-5), cytomegalovirus (CMV), Hepatitis B virus (HBV), Hepatitis C virus (HCV), Epstein-Barr virus (EBV), an adenovirus, human immunodeficiency virus (HIV), human T cell lymphotrophic virus (HTLV-1), an influenza virus, RSV, HPV, rabies, mumps rubella virus, poliovirus, yellow fever, hepatitis A, hepatitis B, Rotavirus, varicella virus, human papillomavirus (HPV), smallpox, zoster, and combinations thereof. Peptides derived from SARS-CoV2 are of interest, and are provided in Tables 2A, 2B and 3. In some embodiments a peptide is a PCPS peptide.

In some embodiments, the bacteria is selected from the group consisting of: Klebsiella spp., Mycobacterium leprae, Mycobacterium lepromatosis, and Mycobacterium tuberculosis. In some embodiments, the bacteria is selected from the group consisting of: typhoid, pneumococcal, meningococcal, haemophilus B, anthrax, tetanus toxoid, meningococcal group B, cholera, and combinations thereof.

In some embodiments, the parasite is a helminth or a protozoan. In some embodiments, the parasite is selected from the group consisting of: Leishmania spp. (e.g. L. major, L. infantum, L. braziliensis, L. donovani, L. chagasi, L. mexicana), Plasmodium spp. (e.g. P. falciparum, P. vivax, P. ovale, P. malariae), Trypanosoma cruzi, Ascaris lumbricoides, Trichuris trichiura, Necator americanus, and Schistosoma spp. (S. mansoni, S. haematobium, S. japonicum).

In some embodiments, an immunogenic antigen composition or vaccine is selected based on TCRs identified in a subject. In one embodiment, identifying a T cell repertoire and testing it in functional assays is used to determine an immunogenic composition or vaccine to be administered to a subject with a condition or disease. In some embodiments, the immunogenic composition is an antigen vaccine. In some embodiments, the antigen vaccine comprises subject specific antigen peptides. In some embodiments, antigen peptides to be included in an antigen vaccine are selected based on a quantification of subject specific TCRs that bind to the antigens. In some embodiments, antigen peptides are selected based on a binding affinity of the peptide to a TCR. In some embodiments, the selecting is based on a combination of both the quantity and the binding affinity. For example, a TCR that binds strongly to an antigen in a functional assay but is not highly represented in a TCR repertoire can be a good candidate for an antigen vaccine because T cells expressing the TCR would be advantageously amplified.

The methods described herein can involve adoptive transfer of immune system cells, such as T cells, specific for selected antigens, such as tumor or pathogen associated antigens. Various strategies can be employed to genetically modify T cells by altering the specificity of the T cell receptor (TCR), for example by introducing new TCRa- and β-chains with specificity to an immunogenic antigen peptide identified using methods known in the art.

Cell therapy methods can also involve the ex vivo activation and expansion of T cells. In some embodiments, T cells can be activated before administering them to a subject in need thereof. Examples of these type of treatments include the use tumor infiltrating lymphocyte (TIL) cells (see U.S. Pat. No. 5,126,132), cytotoxic T cells (see U.S. Pat. Nos. 6,255,073; and 5,846,827), expanded tumor draining lymph node cells (see U.S. Pat. No. 6,251,385), and various other lymphocyte preparations (see U.S. Pat. Nos. 6,194,207; 5,443,983; 6,040,177; and 5,766,920).

An ex vivo activated T cell population can be in a state that maximally orchestrates an immune response to cancer, infectious diseases, or other disease states, e.g., an autoimmune disease state. For activation, at least two signals can be delivered to the T cells. The first signal is normally delivered through the T cell receptor (TCR) on the T cell surface. The TCR first signal is normally triggered upon interaction of the TCR with peptide antigens expressed in conjunction with an MHC complex on the surface of an antigen-presenting cell (APC). The second signal is normally delivered through co-stimulatory receptors on the surface of T cells. Co-stimulatory receptors are generally triggered by corresponding ligands or cytokines expressed on the surface of APCs.

T cells specific to immunogenic antigen peptides identified using the method described herein can be obtained and used in methods of treating or preventing disease. In this regard, the disclosure provides a method of treating or preventing a disease or condition in a subject, comprising administering to the subject a cell population comprising cells specific to immunogenic antigen peptides identified using the method described herein in an amount effective to treat or prevent the disease in the subject. In some embodiments, a method of treating or preventing a disease in a subject, comprises administering a cell population enriched for disease-reactive T cells to a subject in an amount effective to treat or prevent cancer in the mammal. The cells can be cells that are allogeneic or autologous to the subject.

The disclosure further provides a method of inducing a disease specific immune response in a subject, vaccinating against a disease, treating and/or alleviating a symptom of a disease in a subject by administering the subject an antigenic peptide or vaccine.

The peptide or composition of the disclosure can be administered in an amount sufficient to induce a CTL response. An antigenic peptide or vaccine composition can be administered alone or in combination with other therapeutic agents. Exemplary therapeutic agents include, but are not limited to, a chemotherapeutic or biotherapeutic agent, radiation, or immunotherapy. Any suitable therapeutic treatment for a particular disease can be administered. Examples of chemotherapeutic and biotherapeutic agents include, but are not limited to, Abitrexate (Methotrexate Injection), Abraxane (Paclitaxel Injection), Adcetris (Brentuximab Vedotin Injection), Adriamycin (Doxorubicin), Adrucil Injection (5-FU (fluorouracil)), Afinitor (Everolimus), Afinitor Disperz (Everolimus), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Bexxar (Tositumomab), BiCNU (Carmustine), Blenoxane (Bleomycin), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Dacogen (Decitabine), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Eligard (Leuprolide), Ellence (Ellence (epirubicin)), Eloxatin (Eloxatin (oxaliplatin)), Elspar (Asparaginase), Emcyt (Estramustine), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leukeran (Chlorambucil), Leukine (Sargramostim), Leustatin (Cladribine), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Prednisone Intensol (Prednisone), Proleukin (Aldesleukin), Purinethol (Mercaptopurine), Reclast (Zoledronic acid), Revlimid (Lenalidomide), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Trelstar (Triptorelin Injection), Trexall (Methotrexate), Trisenox (Arsenic trioxide), Tykerb (lapatinib), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (lbritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MED14736, MPDL3280A and MSB0010718C targeting PD-L1 and those targeting CTLA-4 such as ipilimumab.

The amount of each peptide to be included in a vaccine composition and the dosing regimen can be determined by one skilled in the art. For example, a peptide or its variant can be prepared for intravenous (i.v.) injection, sub-cutaneous (s.c.) injection, intradermal (i.d.) injection, intraperitoneal (i.p.) injection, intramuscular (i.m.) injection. Exemplary methods of peptide injection include s.c, i.d., i.p., i.m., and i.v. Exemplary methods of DNA injection include i.d., i.m., s.c, i.p. and i.v. Other methods of administration of the vaccine composition are known to those skilled in the art.

A pharmaceutical composition can be compiled such that the selection, number and/or amount of peptides present in the composition is/are disease and/or patient-specific. For example, the exact selection of peptides can be guided by expression patterns of the parent proteins in a given tissue to avoid side effects. The selection can be dependent on the specific type of disease, the status of the disease, earlier treatment regimens, the immune status of the patient, and the HLA-haplotype of the patient. Furthermore, the vaccine according to the present disclosure can contain individualized components, according to personal needs of the particular patient. Examples include varying the amounts of peptides according to the expression of the related antigen in the particular patient, unwanted side-effects due to personal allergies or other treatments, and adjustments for secondary treatments following a first round or scheme of treatment.

### Identification Methods

In some embodiments, a method is provided for the rapid identification of CD8⁺ T cell epitopes. The methods comprise incubation of a candidate protein of interest with activated 20S immunoproteasome and a molar excess of PA28 activator alpha subunit protein for a period of time sufficient to digest the protein, e.g. from about 12 to about 36 hours, and may be around 24 hours. Candidate proteins of greater than about 50 amino acids may be pre-treated by denaturation, e.g. by incubation in urea, prior to digestion with the immunoproteasome. The proteasome digest is then immunoprecipitated with Class I MHC molecules, e.g. human HLA-A, B, C Class I molecules, for example by incubation with a substrate comprising immobilized HLA proteins, followed by washing the substrate free of unbound peptides. The bound peptides can then be eluted and analyzed for molecular weight, sequencing, mass spectrometric methods such as MALDI-ToF or LC-MS/MS, and/or de novo sequencing by mass spectrometry or chemical sequencing such as by Edman degradation.

The sequence identity of the proteasome derived fragments can be determined using LC-MS/MS combined with a database containing potential linear and PCPS fragments derived from the protein of interest.

In such de novo sequencing of the eluted peptides, a computer algorithm matches the molecular weight, for example as determined by mass spectrometry, with the same molecular weights from the known sequences in a database. A peptide can be matched across the b- and y-series of fragments to a known sequence. While co-linear (or contiguous) peptides are efficiently identified by this method, such matches are more difficult for spliced (PCPS) peptides. Provided herein are methods for generating a database useful in matching spliced peptides.

De novo peptide sequencing is a method for peptide sequencing performed without prior knowledge of the amino acid sequence. It uses computational approaches to deduce the sequence of peptide directly from the experimental MS/MS spectra. It can be used for un-sequenced organisms, antibodies, peptides with posttranslational modifications (PTMs), and endogenous peptides.

In this method, a peptide is fragmented along the peptide backbone and the resulting fragment ions are measured to produce spectra. There are 3 ways to break bonds to form peptide fragment: alkyl carbonyl (CHR—CO), peptide amide bond (CO—NH), and amino alkyl bond (NH—CHR). Therefore, it can form 6 types of fragmentation ions, including the N-terminal charged fragment ions which are classed as a, b, or c, and the C-terminal charged ones which are classed as x, y, or z. And because the peptide amide bone (CO—NH) is the most vulnerable, the most common peptide fragments observed in low energy collisions are a, b and y ions.

De novo methods use the knowledge of the fragmentation methods employed in the MS. CID, Collision induced dissociation, also known as collisionally activated dissociation, is the most common form of fragmentation. The ions can obtain high kinetic energy and collide with neutral molecules. Some of the kinetic energy is converted into internal energy which leads to bond breakage and the fragmentation of the molecules into smaller fragments. This method results in the formation of b and y series ions from the precursor ion. The Electron capture dissociation (ECD) and Electron transfer dissociation (ETD) have been implemented in the recent mass spectrometer. In these methods, ions are fragmented after reaction with electrons. After fragmentation, it forms c and z type ions through cleavage of the peptide bond between the amino group and alpha carbon.

The mass can usually uniquely determine the residue. The main principle of de novo sequencing is to use the mass difference between two fragment ions to calculate the mass of an amino acid residue on the peptide backbone. For example, the mass difference between the y7 and y6 ions in the following figure is equal to 101, which is the mass of residue T. Thus, if one can identify either the y-ion or b-ion series in the spectrum, the peptide sequence can be determined. However, the spectrum obtained from the mass spectrometry instrument does not tell the ion types of the peaks, which need an expert or a computer algorithm to figure out. There a number of commercially available software packages used for de novo sequencing

Three approaches are provided for PCPS database construction. In one embodiment to generate a database for a peptide-spectra match (PSM) search, the molecular weights observed through MALDI-TOF are aggregated. Taking the range of observed original molecular weights, the potential linear and PCPS-derived rearrangements of the parental sequence that match the original weight molecular weight are calculated, where the peptides are restricted to lengths between about 8 and about 12 amino acid residues in length. This algorithm is used to generate a FASTA database of co-linear and PCPS spliced peptides that are used for de novo sequencing by MS/MS. In some embodiments, this method is applied to peptides of less than about 100 aa in length, or less than about 50 aa in length.

In other embodiments involving larger proteins, e.g. greater than about 50 aa in length, greater than about 100 aa in length, or more, molecular weight data from MALDI-ToF is used to generate a database of the co-linear fragments from a proteasome digestion. These co-linear fragments are directly derived from the parental protein. From this data, the fragments from 2 to 12 aa are used to construct a database that encompasses possible PCPS recombinants. A boundary may be set on the distance of cis-ligation, e.g. more than 1 aa, less than about 50 aa, less than about 30 aa distance, between the sites of splicing. Boundaries on the peptide size in the database may be set at from about 8 to about 12 amino acids in length. An algorithm is then used to assemble a database where 2-10 aa fragments across any given 50 aa window are used in combinatorial fashion to make hypothetical PCPS sequences of between 8-12 aa, containing no more than 3 fragments. Since the sequence of the parental protein is known, a combination of software, which takes into account ionization status, Na+ ions, and other potential modifications can be used to determine the sequence of co-linear fragments between 200 to 1400 Da.

Because PCPS database construction in the first two methods requires experimental identification of fragments, an algorithm dependent only on the protein sequence was developed. In some embodiments a universal database is developed where a protein sequence can be plugged in generate the actual fragments. In this embodiment, all possible windows of from 2 to 12 amino acids across a sequence of about 50 to about 70 amino acids are generated. This list of sequences is used to generate all possible recombinants of from 1 to 3 fragments that range from 8-12 aa. Redundant sequences are pruned. To continue walking across the entire protein sequence, peptide windows containing the first amino acid are eliminated and windows including the next amino acid in the sequence are added. Because the vast majority of recombinations will be the same, it is only necessary to contemplate the recombinations arising from the new amino acid.

Once co-linear and PCPS fragments that bind to HLA-I are identified through a matching algorithm to a database as disclosed above, the peptides are optionally confirmed for binding to an appropriate class I MHC in an MHC stabilization assay. Such assays include, without limitation, incubation of a candidate peptide with a defective transporter associated with antigen processing (TAP) cell lines, such as T2 (human) or RMA-S (mouse) and a cell expressed the targeted MHC class I protein, where the presence of a stabilized MHC protein/peptide complex can be detected by any convenient method. Peptides can also be subjected to functional assays, e.g. determining induction of a T cell response; binding to CD8+ T cells, and the like as appropriate for the peptide.

Also provided herein are software products tangibly embodied in a machine-readable medium, the software product comprising instructions operable to cause one or more data processing apparatus to perform operations comprising: generating a n×20 matrix from the positional frequencies of selected peptide ligands obtained by the screening methods of the invention, where n is the number of amino acid positions in the peptide ligand library. A cutoff of amino acid frequencies is set, e.g. less than 0.1, less than 0.05, less than 0.01, and frequencies below the cutoff are set to zero. A database of sequences, e.g. a set of human polypeptide sequences; a set of pathogen polypeptide sequences, a set of microbial polypeptide sequences, a set of allergen polypeptide sequences; etc. are searched with the algorithm using an n-position sliding window alignment with scoring the product of positional amino acid frequencies from the substitution matrix. An aligned segment containing at least one amino acid where the frequency is below the cutoff is excluded as a match. The results of the search can be output as a data file in a computer readable medium

The peptide sequence results and database search results may be provided in a variety of media to facilitate their use. “Media” refers to a manufacture that contains the expression repertoire information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

Methods and systems of the present disclosure can be implemented by way of one or more algorithms. An algorithm can be implemented by way of software upon execution by the central processing unit. The algorithm can, for example, input amino acid position information, transfer imputed information into datasets, and generate a trained algorithm with the datasets.

The present disclosure provides computer control systems that are programmed to implement methods of the disclosure. A computer system is programmed or otherwise configured to train a machine-learning HLA-peptide presentation prediction model. The computer system can regulate various aspects of the present disclosure, such as, for example, inputting amino acid position information, transferring imputed information into datasets, and generating a trained algorithm with the datasets. The computer system can be a user electronic device or a remote computer system. The electronic device can be a mobile electronic device.

The computer system includes a central processing unit (CPU, also “processor” and “computer processor” herein), which can be a single core or multi core processor, either through sequential processing or parallel processing. The computer system also includes a memory unit or device (e.g., random-access memory, read-only memory, flash memory), a storage unit (e.g., hard disk), a communication interface (e.g., network adapter) for communicating with one or more other systems, and peripheral devices, either external or internal or both, such as a printer, monitor, USB drive and/or CD-ROM drive. The memory, storage unit, interface and peripheral devices are in communication with the CPU through a communication bus, such as a motherboard. The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network.

The CPU can execute a sequence of machine-readable instructions, which can be embodied in a program or software. The instructions may be stored in memory. The instructions can be directed to the CPU, which can subsequently program or otherwise configure the CPU to implement methods of the present disclosure. Examples of operations performed by the CPU can include fetch, decode, execute, and writeback.

Methods as described herein can be implemented by way of machine (e.g., computer processor) executable code stored on an electronic storage location of the computer system, such as, for example, in memory or a data storage unit. The machine executable or machine readable code can be provided in the form of software. During use, the code can be executed by the processor. In some cases, the code can be retrieved from the storage unit and stored in memory for ready access by the processor. In some situations, the storage unit can be precluded, and machine-executable instructions are stored in memory.

The code can be pre-compiled and configured for use with a machine having a processor adapted to execute the code, or it can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on a storage unit, such as a hard disk, or in memory (e.g., read-only memory, random-access memory, flash memory). “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for providing, for example, probability that one or more proteins encoded by a class I MHC allele of a cancer cell of the subject will present a given sequence of a peptide sequence identified. Examples of UI's include, without limitation, a graphical user interface (GUI) and web-based user interface.

The search algorithm and sequence analysis may be implemented in hardware or software, or a combination of both. In one embodiment of the invention, a machine-readable storage medium is provided, the medium comprising a data storage material encoded with machine readable data which, when using a machine programmed with instructions for using said data, is capable of displaying any of the datasets and data comparisons of this invention. In some embodiments, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language may be a compiled or interpreted language. Each such computer program can be stored on a storage media or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

Further provided herein is a method of storing and/or transmitting, via computer, sequence, and other, data collected by the methods disclosed herein. Any computer or computer accessory including, but not limited to software and storage devices, can be utilized to practice the present invention. Sequence or other data can be input into a computer by a user either directly or indirectly. Additionally, any of the devices which can be used to sequence DNA or analyze DNA or analyze peptide binding data can be linked to a computer, such that the data is transferred to a computer and/or computer-compatible storage device. Data can be stored on a computer or suitable storage device (e.g., CD). Data can also be sent from a computer to another computer or data collection point via methods well known in the art (e.g., the internet, ground mail, air mail). Thus, data collected by the methods described herein can be collected at any point or geographical location and sent to any other geographical location.

Also provided are reagents and kits thereof for practicing one or more of the above-described methods. The subject reagents and kits thereof may vary greatly. Reagents of interest include reagents specifically designed for use in the methods of the invention. In some embodiments the kit will further comprise a software package for analysis of a sequence database.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The above-described analytical methods may be embodied as a program of instructions executable by computer to perform the different aspects of the invention. Any of the techniques described above may be performed by means of software components loaded into a computer or other information appliance or digital device. When so enabled, the computer, appliance or device may then perform the above-described techniques to assist the analysis of sets of values associated with a plurality of peptides in the manner described above, or for comparing such associated values. The software component may be loaded from a fixed media or accessed through a communication medium such as the internet or other type of computer network. The above features are embodied in one or more computer programs may be performed by one or more computers running such programs.

Software products (or components) may be tangibly embodied in a machine-readable medium, and comprise instructions operable to cause one or more data processing apparatus to perform operations comprising: a) clustering sequence data from a plurality of immunological receptors or fragments thereof; and b) providing a statistical analysis output on said sequence data. Also provided herein are software products (or components) tangibly embodied in a machine-readable medium, and that comprise instructions operable to cause one or more data processing apparatus to perform operations comprising: storing and analyzing sequence data.

### EXPERIMENTAL

The following examples are offered by way of illustration and not by way of limitation.

### Example 1

### Enhancing Proteasomal Processing Significantly Improves Survival for a Peptide Vaccine Used to Treat Glioblastoma

Despite its essential role in antigen presentation, enhancing proteasomal processing is an unexploited strategy for improving vaccines. pepvIII, an anti-cancer vaccine targeting EGFRvIII, has been tested in several trials for glioblastoma. We examined 20 peptides in silico and experimentally which showed a tyrosine substitution (Y6-pepvIII) maximizes proteasome cleavage and survival in a subcutaneous tumor model. In an intracranial glioma model, Y6-pepvIII showed a 62% and 31% improvement in median survival vs. control and pepvIII vaccinated mice. Y6-pepvIII vaccination uniquely altered TIL subsets and expression of PD-1 on intratumoral T-cells. Combination anti-PD-1 therapy cured 45% of the Y6-pepvIII vaccinated mice but was ineffective for pepvIII treated mice. LC-MS/MS analysis of proteasome digested pepvIII and Y6-pepvIII revealed most fragments are similar but more abundant in Y6-pepvIII digests. Interestingly, 77% result from proteasome catalyzed peptide splicing (PCPS). We identified 10 peptides that bound human and murine MHC-Class I. Significantly, nine are PCPS products and only one peptide is co-linear with EGFRvIII indicating that PCPS fragments may be a significant component of MHC Class I recognition. Interestingly, despite not being co-linear with EGFRvIII, 2 of 3 PCPS products tested are capable of increasing survival when administered independently as vaccines. We hypothesize that the immune response to a vaccine represents the collective contribution from multiple PCPS and linear products. Our work demonstrates a strategy to increase proteasomal processing of a vaccine which results in an augmented immune response and enhanced survival. These findings and strategy could be relevant to increasing the effectiveness of any vaccine.

Glioblastoma (GBM) remains one of the most intractable tumors to treat despite significant advances in understanding its basic biology. Only a handful of approaches have been approved to treat GBM in the past 20 years, each producing only a 2-3-month improvement in survival, and even with the most aggressive approaches, tumors invariably recur. The toxicity and/or the imprecision of current treatment strategies often represent the critical limitation. Immunotherapy offers a potentially precise approach to eliminate tumor cells without the destruction of normal tissue that limits the efficacy of chemo and radiation therapies.

For GBM, there is a particularly intriguing target. EGFRvIII results from an in-frame deletion in the EGF receptor gene that juxtaposes exons 1 and 8 with the creation of a novel glycine at the junction. This produces a constitutively active, tumor specific protein found in ˜30% of patients. Aside from being overexpressed and oncogenic, it is present in cancer stem cells and is highly immunogenic. Thus, it possesses many ideal qualities for an immunotherapeutic target. Chimeric antigen receptor (CAR) T cells directed against EGFRvIII have shown promise in a Phase I study for GBM. While this study further validated EGFRvIII as a relevant target in the treatment of GBM, clinical utilization of CAR-T cells remains costly and time consuming. On the other hand, synthetic peptide vaccines are relatively inexpensive to produce and simple to administer. PepvIII is a 13-amino acid peptide (SEQ ID NO:413) (LEEKKGNYVVTDH) that spans the exon 1 to 8 junction and novel glycine in EGFRvIII—the complete drug has an additional cysteine at the C-terminus for conjugation to the carrier protein KLH. It has shown considerable promise in several preclinical studies and three Phase II trials. PepvIII conjugated to KLH was recently evaluated in a Phase Ill clinical trial (ACT IV) for newly diagnosed GBM patients; this study was terminated early after an interim analysis demonstrated no significant difference in overall-survival between treatment groups for patients with <2 cm³ residual tumor post resection, although there was a statistically significant improvement in OS for patients with >2 cm³ residual tumors. Moreover, a randomized Phase II trial (ReACT) of pepvIII in combination with bevacizumab also showed a significant increase in OS vs. patients receiving bevacizumab plus KLH alone, showing an EGFRvIII targeting vaccine may have clinical utility when applied in the correct context.

In light of the potential of pepvIII, it is important to note that the pepvIII peptide sequence never underwent any further optimization to enhance immunologic properties. While there have been substantial advances in delivery, adjuvants, co-stimulatory molecules and the addition of checkpoint inhibitors, there have not been any significant advances in ways to enhance the immunizing peptide itself. The previous strategies that have been attempted include epitope enhancement to increase binding to either Class I or II molecules or the T cell receptor or mimotopes to mimic peptide epitopes. While the mutated peptides do show strong binding, this has not translated into improved patient responses. A key event in antigen presentation that has been largely overlooked is processing by the proteasome. The proteasome is responsible for the degradation of self and non-self-proteins into peptide antigens that will be presented to T cells in the context of MHC molecules and it is the resulting pattern of these cleaved peptides that determines the immune response. Characteristics such as amino acid sequence and tertiary structure influence proteasomal processing and subsequent antigen presentation. It has been demonstrated that the proteasome can also rearrange and ligate cleaved peptides through a mechanism called proteasome catalyzed peptide splicing (PCPS), thus creating novel antigens that can bind to MHC molecules and stimulate T cell mediated lysis. It has been estimated that up to 20-30% of the Class I presented peptides are derived from PCPS. Enhancing proteasomal processing may enhance subsequent immune responses. In the case of pepvIII, evaluation of the tertiary peptide structure revealed that the GLY-6 residue protrudes from the molecule and forms a type-II p turn which may impair proteasomal processing. We hypothesized that substitution of this amino acid would facilitate enhanced proteasomal processing and ultimately increase vaccine efficacy.

### Results

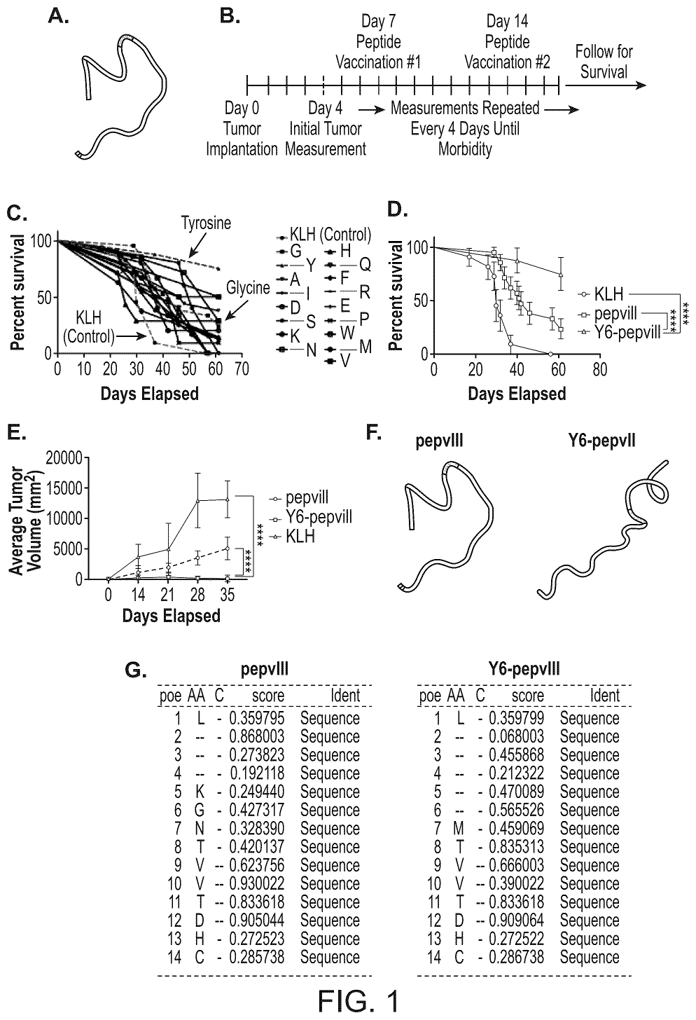

Amino acid substitution of the novel glycine can significantly increase survival. In the EGFRvIII rearrangement, the fusion of exon 1 to exon 8 produces a novel glycine (GLY-6) at the fusion junction. Because it is unique, it might be a key amino acid for immune recognition but the 1.8 Å resolution crystal structure of pepvIII complexed with a scFv antibody revealed no contacts with the glycine residue itself. The GLY-6 residue does, however, facilitate the formation of a tight β turn that has a dramatic effect on the peptide tertiary structure (FIG. 1A). The immunoproteasome preferentially cleaves peptides after polar rather than relatively non-polar residues such as glycine. The direct trans-peptidation model of proteasomal processing suggests that more proteasomal cleavage products are related to increased proteasomal processing and potentially more robust antigen presentation. Considering the impact of the GLY-6 on peptide structure in combination with known cleavage preferences of the immunoproteasome, we elected to explore substitutions of this amino acid and their effect on survival in a subcutaneous tumor model.

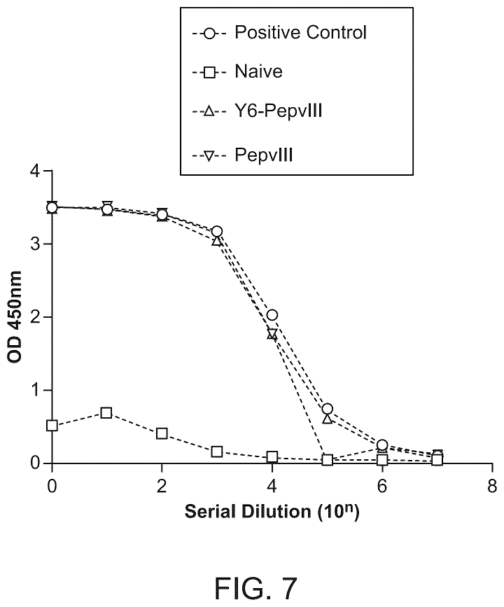

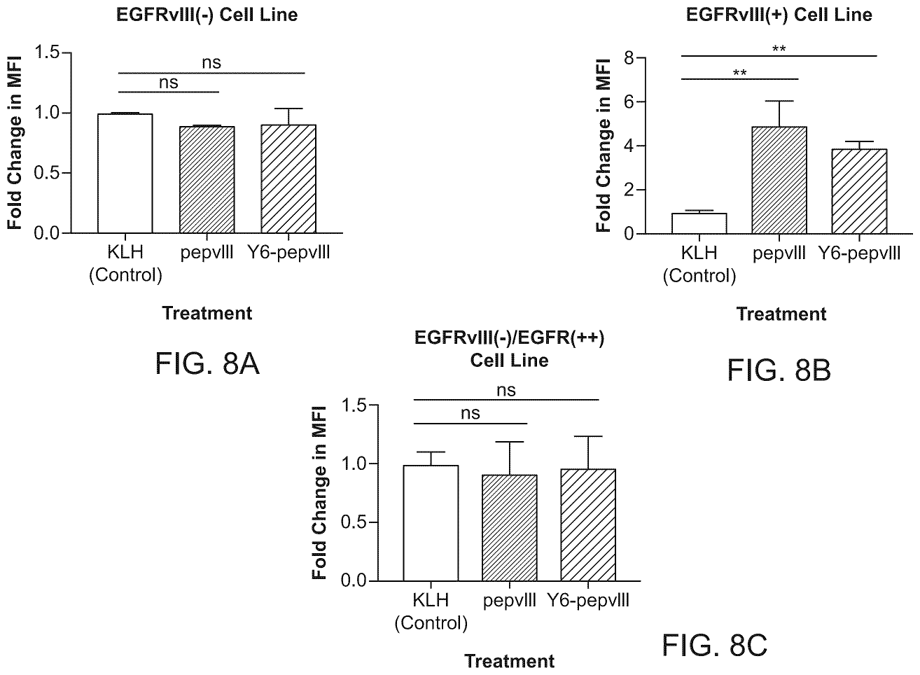

We synthesized peptides that replaced GLY-6 with all other canonical amino acids which were then conjugated to KLH. In addition, we tested lengthening the peptide by 1 or 2 amino acids on both the N- and C-terminus, and a peptide where GLY-6 was deleted. To efficiently analyze such a large number of peptides, a subcutaneous tumor model utilizing EGFRvIII+HC2 20d2/c cells was used. This animal model is nearly identical to the murine model used to support an IND application for pepvIII, with the exception that it was made more rigorous by delaying the first vaccination until after tumors had formed. Following the injection schedule outlined in FIG. 1B, we assessed the in vivo efficacy of each peptide vaccine. The TYR-6 substituted variant (Y6-pepvIII) demonstrated the greatest increase in overall survival as compared to KLH treated controls (FIG. 1C), whereas most vaccines did not have a strong effect. Mice vaccinated with Y6-pepvIII showed statistically significantly longer survival than those that received the original pepvIII or KLH controls (FIG. 1D). In addition, mice vaccinated with Y6-pepvIII demonstrated significantly reduced tumor volume as compared to mice receiving either KLH or pepvIII (FIG. 1E). Using serum from the vaccinated mice, we further examined antibody titers and the recognition of EGFRvIII by ELISA and flow cytometry (FIGS. 7 and 8). Nearly all vaccinated mice had similarly high titers against EGFRvIII and produced antibodies recognizing EGFRvIII on Western blots, indicating there was no strong correlation between the humoral response and survival.

Computational modeling demonstrates that Y6-pepvIII no longer exhibits the central β-turn structure of pepvIII (FIG. 1F). We used the program NetChop to examine how amino acid substitutions would affect proteasomal cleavage. Among all amino acids, tyrosine shows the greatest increase in cleavage score (FIG. 1G and Supplementary Table 1). This peptide also shows the overall greatest increase in score for more distant amino acids (Supplementary Table 1) suggesting that the change in secondary structure facilitates cleavage throughout the peptide. We then performed proteasome digests using peptides with substitutions of all other amino acids for GLY-6. This confirmed that Y6-pepvIII undergoes the most extensive proteasomal processing of all 20 amino acid substitutions tested (Supplementary Table 2).

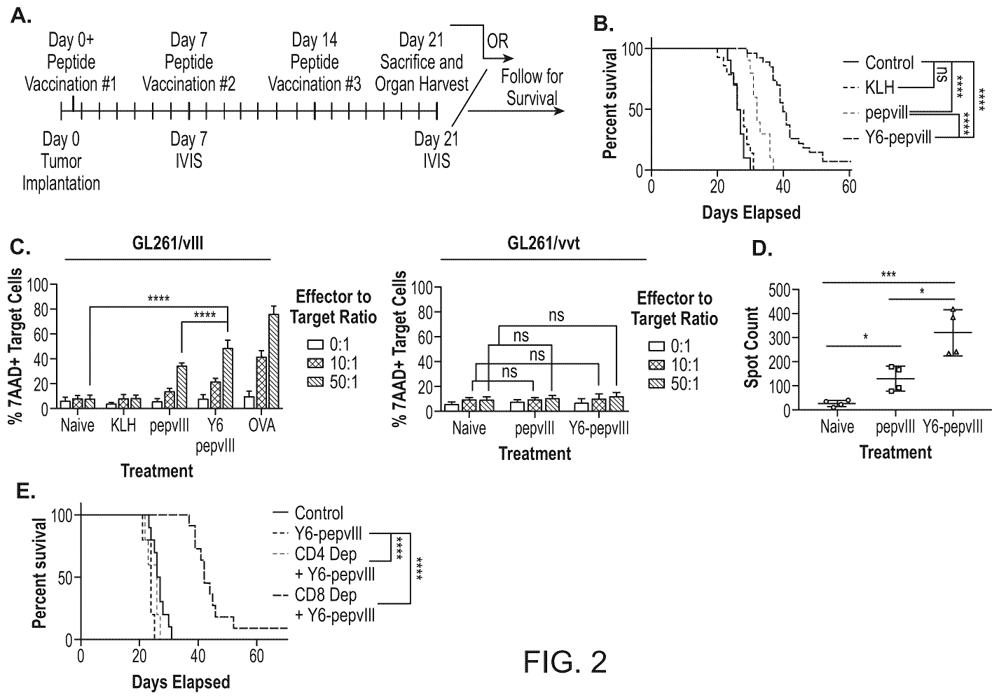

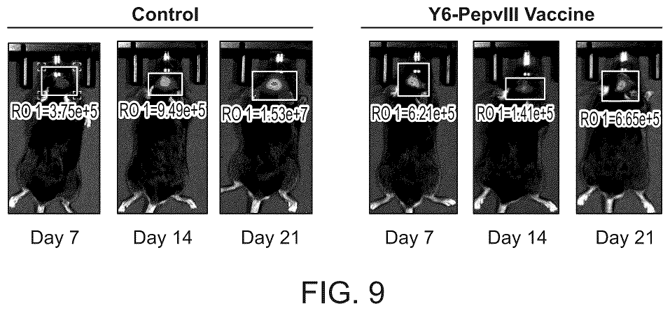

Y6-pepvIII is effective in an intracranial glioma model. Because of its significant effect on survival in the EGFRvIII subcutaneous tumor model, we selected Y6-pepvIII for in-depth analysis. First, we tested this vaccine in a model that more accurately represented human gliomas. We utilized the commonly employed murine glioma 261 (GL261) intracranial tumor model (30) which was transfected with the EGFRvIII cDNA to yield a line with high expression, GL261/vIII. We explored intracranial injection protocols that would lead to reproducible survival curves and identified conditions where 90% of the animals survived 23-29 days. To compare vaccine efficacy, mice received either Montanide (ISA51) alone, 100 ug of KLH with Montanide, 100 ug of KLH conjugated to pepvIII or Y6-pepvIII in Montanide following the vaccination schedule outlined in FIG. 2A. While KLH treatment had no measurable impact on survival, vaccination with the pepvIII induced a modest but statistically significant 23% increase in survival vs. control mice (FIG. 2B; median survival: Control=26.0 days, KLH=27 days, pepvIII=32 days, p<0.0001). Conversely, vaccination with the Y6-pepvIII significantly increased survival by 62% vs. control mice and 31% vs. pepvIII vaccinated mice (FIG. 2B; median survival: Y6-pepvIII=42 days, p<0.0001, FIG. 9).

Survival benefit of the Y6-pepvIII vaccine is dependent on both CD4+ and CD8+ T cells. An effective peptide vaccine must be capable of eliciting, expanding and activating tumor specific T cells. To assess the ability of the Y6-pepvIII vaccine to induce T cell specific immune responses, we performed a cytotoxic T cell killing assay in which we compared the capacity of T cells isolated from mice vaccinated with either pepvIII or Y6-pepvIII peptide to kill GL261/vIII target cells in vitro. The cytolytic activity of these activated T cells was compared to naïve mice and OVA vaccinated mice as a positive control. As demonstrated by FIG. 2C, both pepvIII and Y6-pepvIII vaccination elicited T cells that killed GL261/vIII target cells in vitro (FIG. 2C, Left Panel). Interestingly, T cells from the Y6-pepvIII vaccinated mice showed an increased cytotoxic capacity at the 50:1 effector to target ratio (FIG. 2C, Left Panel).

Importantly, T cells derived from both pepvIII and Y6-pepvIII vaccinated mice showed no cytotoxic activity against GL261/wt target cells which do not express EGFRvIII, confirming the antigen specificity of the Y6-pepvIII vaccine response (FIG. 2C, Right Panel). To further confirm differential activation of T cells, we performed an ELIspot assay to assess the cytokine production capacity of splenic T cells derived from pepvIII and Y6-pepvIII vaccinated mice.

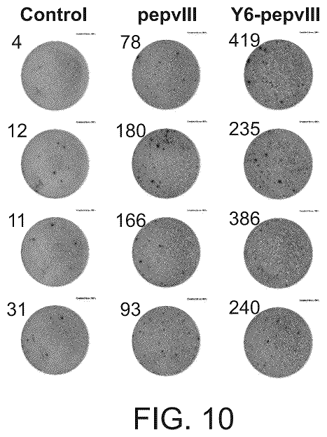

PepvIII and Y6-pepvIII vaccination induced statistically significant increases in IFNγ producing T cells relative to control (KLH treated mice). A modest but statistically significant difference was also observed between pepvIII and Y6-pepvIII vaccination. (FIG. 2D, FIG. 10). To confirm the role of T cells in the anti-tumor response, we assessed the efficacy of Y6-pepvIII vaccination in tumor bearing mice that had either the CD4+ or CD8+ T cell populations depleted prior to vaccination. In the absence of either CD4+ or CD8+ T cells, the Y6-pepvIII peptide vaccine had no effect on survival, indicating that both CD4+ and CD8+ T cell subsets are involved in the anti-tumor response (FIG. 2E).

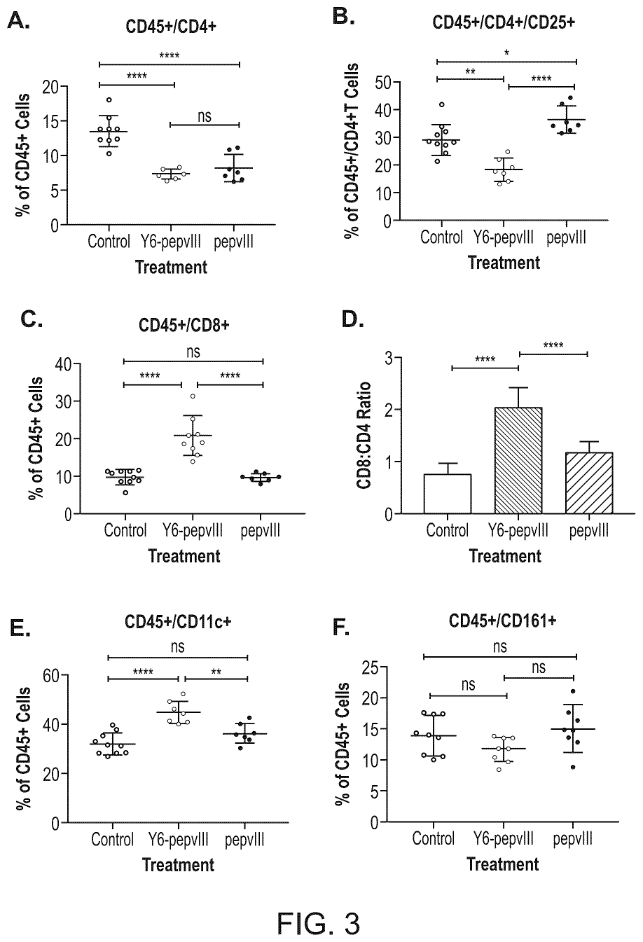

Vaccination with the Y6-pepvIII increases the proportion of intratumoral CD8+ T cells. Because vaccine efficacy is dependent on T cells, we sought to further characterize the cell mediated anti-tumor response induced by Y6-pepvIII vaccination. Both pepvIII and Y6-pepvIII vaccines induced a significant reduction in the relative proportion of tumor infiltrating CD3+/CD4+ cells as a proportion of tumor infiltrating CD45+ cells (FIG. 3A). The proportion of tumor infiltrating CD3+/CD4+ that were CD25+ was significantly lower in tumors derived from mice that received the Y6-pepvIII vaccine relative to either control or pepvIII vaccinated mice (FIG. 3B). Vaccination with the Y6-pepvIII peptide induced a significant increase in the CD3+/CD8+ cell population relative to control mice (FIG. 3C). This increase was not observed in mice that received the pepvIII vaccine. In line with these findings, Y6-pepvIII vaccinated mice showed an increased intratumoral CD8:CD4 ratio relative to both control and pepvIII vaccinated mice. Tumors derived from mice vaccinated with the Y6-pepvIII peptide also showed a significant increase in CD45+/CD11c+ cells, a marker combination used to broadly define dendritic cells (FIG. 3D), but no change was observed in intratumoral NK cell proportions between vaccination groups (FIG. 3E). This demonstrates that a single amino acid change in the vaccination peptide can significantly impact the dynamics of vaccine induced cellular anti-tumor responses.

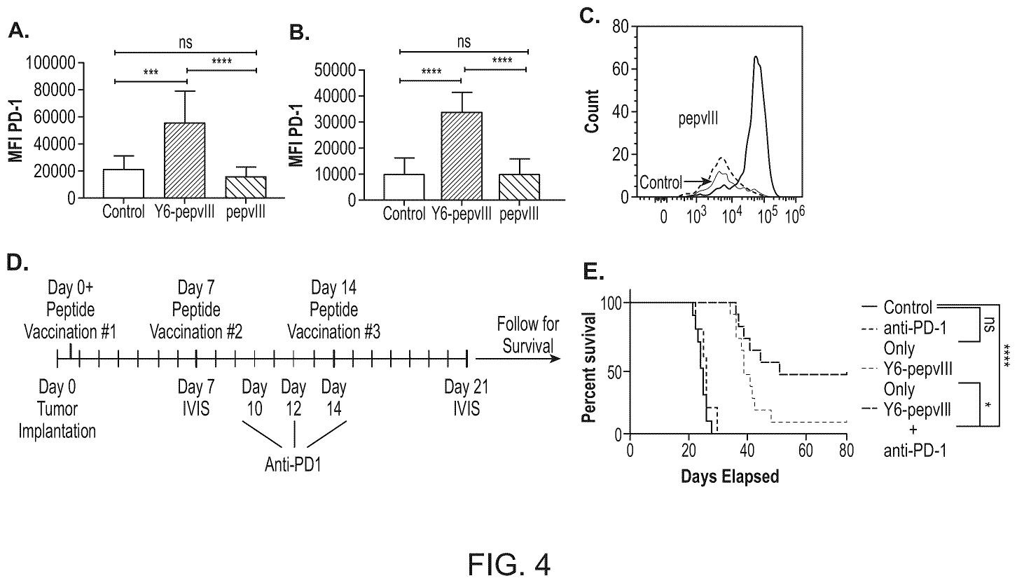

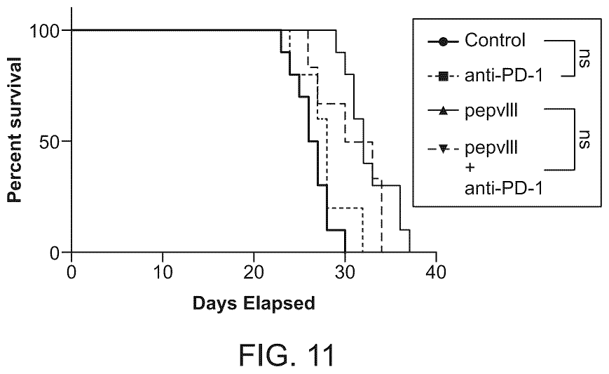

Differential expression of checkpoint molecules induced by vaccination with Y6-pepvIII—the combination with anti-PD-1 therapy dramatically increases survival. While several peptide vaccines have consistently been shown to increase immune cell infiltration of the tumor, the clinical efficacy of these vaccines has been quite variable. An explanation is that the tumor itself can suppress immune recognition through the activation of immune checkpoint pathways. To assess the immune checkpoint pathways relevant in our tumor model, we analyzed the expression of CTLA-4 and PD-1 on the surface of tumor infiltrating CD4+ and CD8+ T cells. We observed no difference in CTLA-4 expression across vaccine cohorts on either CD4+ or CD8+ T cells (data not shown). However, while pepvIII vaccination induced no discernable increase in PD-1 expression on either CD4+(FIG. 4A) or CD8+(FIG. 4B) tumor infiltrating T cells relative to non-vaccinated mice, vaccination with Y6-pepvIII induced significant upregulation of PD-1 on both CD4+(FIG. 4A) and CD8+(FIGS. 4B and 4C) TILs. Considering the strong induction of PD-1 expression on TILs in Y6-pepvIII vaccinated mice, we sought to evaluate the impact of anti-PD-1 therapy in combination with our peptide vaccine. Anti-PD-1 therapy combined with Y6-pepvIII vaccination induced long-term survival (>100 days) in over 45% of recipient mice (FIG. 4E), while Y6-pepvIII vaccination alone resulted in only ˜10% long-term survivors and pepvIII vaccination with or without anti-PD-1 resulted in no long-term survivors (FIG. 11). The combination of anti-PD-1 therapy with Y6-pepvIII vaccination also caused a significant increase in median survival (FIG. 4E) from 42 days to 55 days (p=0.04). Anti-PD-1 therapy by itself (FIG. 4E) conferred no observable survival advantage.

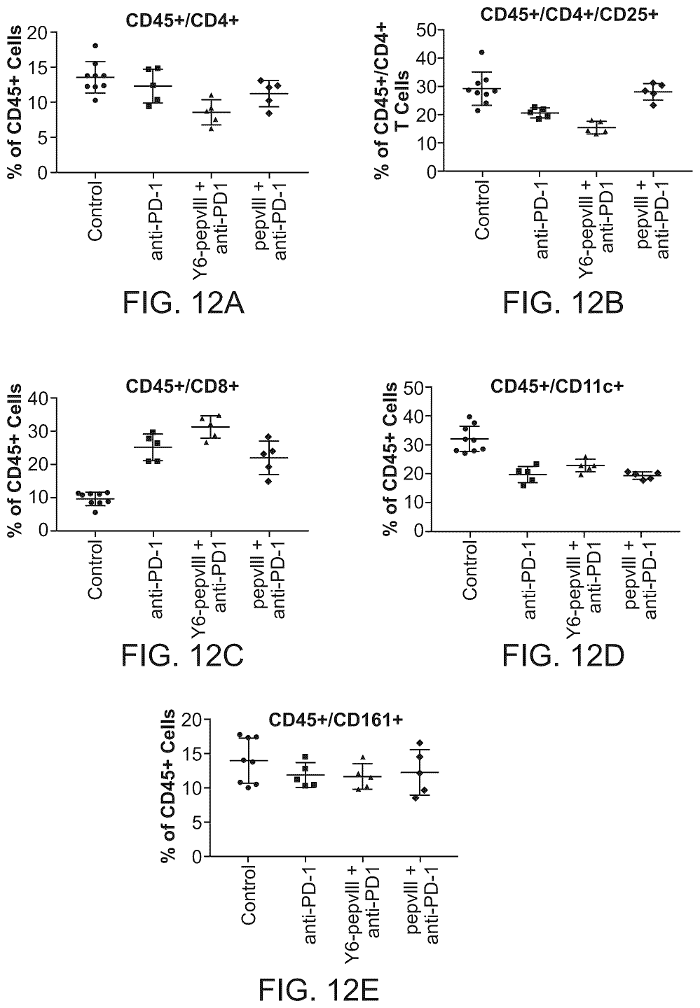

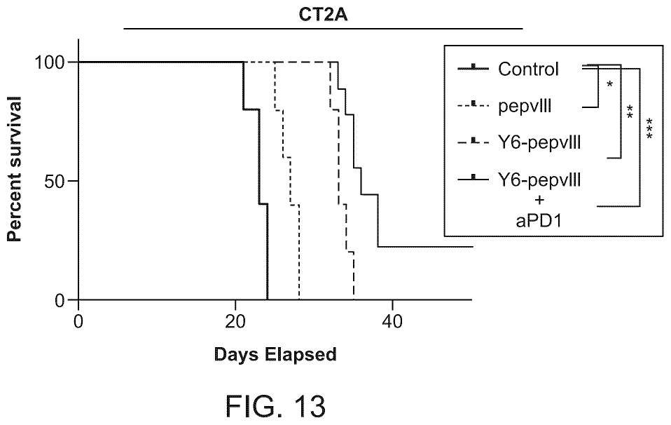

Overall, the addition of anti-PD1 to the peptide vaccine did not have a dramatic impact on intratumoral lymphocyte composition relative to peptide vaccination alone (FIG. 12). Importantly, both the increased efficacy of Y6-pepvIII and the utility of combinational treatment with anti-PD1 were confirmed in a second distinct intracranial tumor model utilizing an EGFRvIII+ derivative of the CT-2A cell line (FIG. 13). Collectively, these experiments demonstrate that our substituted peptide vaccine can also enhance the effectiveness of other immunotherapeutics.

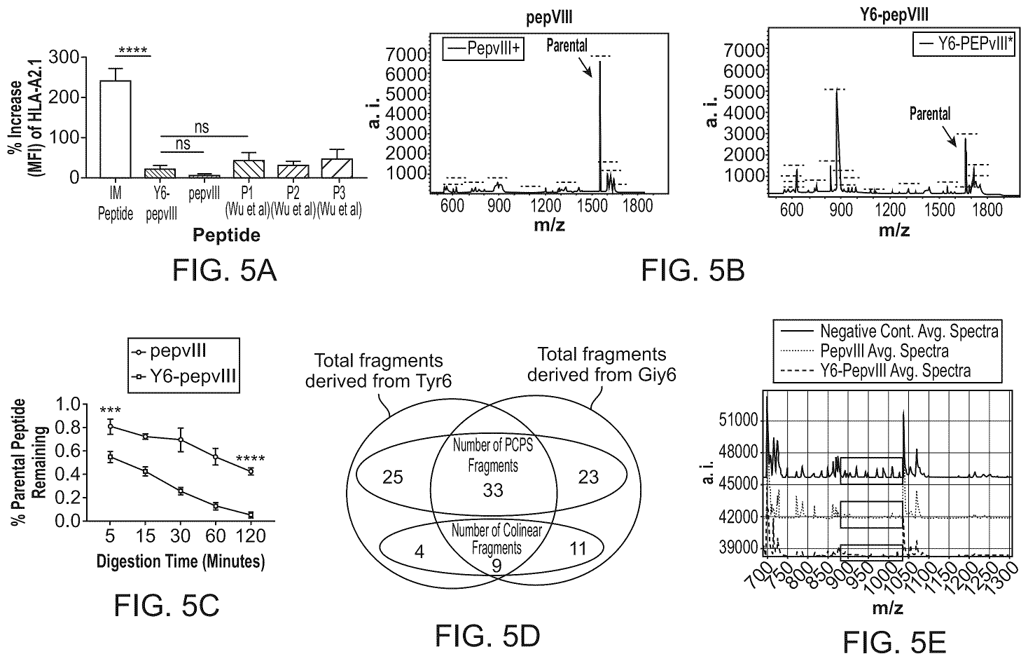

Y6-pepvIII shows an increased rate of proteasomal processing and produces a greater quantity and diversity of potentially antigenic peptides. We sought an explanation for the difference in the anti-tumor response induced by vaccination with Y6-pepvIII vs. pepvIII. Analysis of humoral responses did not reveal any significant differences between pepvIII and Y6-pepvIII (FIGS. 7 and 8), so we focused on CD8+ T cell activation. Binding of a peptide to a cognate MHC Class I molecule is required to initiate CD8+ T cell mediated responses. We utilized the MHC Class I stabilization assay (T2 assay) to see if parental Y6-pepvIII or pepvIII showed binding to HLA-A2 molecules. We also evaluated 3 shorter peptides derived from the pepvIII sequence that have previously been assessed in a similar binding assay. While the positive control peptide (Influenza M1) showed strong stabilization of HLA-A2, neither the intact Y6-pepvIII, pepvIII or the 3 shorter peptides identified by Wu et al. showed substantial binding in our assays (FIG. 5A). This data demonstrates that the tyrosine substitution itself does not enhance Class I binding.

We next examined if proteasomal processing products might play a role. First, we digested both pepvIII and Y6-pepvIII using the human 20S immunoproteasome and assessed the degree of parental peptide processing. Using MALDI-TOF we then quantified the fraction of parental peptide remaining across multiple time points following incubation with the 20S immunoproteasome. As demonstrated by the representative spectra in FIG. 5B, after 30 minutes the proportion of parental peptide remaining was significantly lower for Y6-pepvIII. At each time point, Y6-pepvIII was digested more extensively than pepvIII (FIG. 5C), indicating that Y6-pepvIII can be readily processed while pepvIII is relatively resistant to proteasomal digestion. Analysis of these fragments revealed similar numbers of peptide products are generated from each peptide. Y6-pepvIII and pepvIII produce 13 and 20 fragments, respectively, that are linear subsequences of the parental peptides, of which 9 are in common. We further observe 58 and 56 fragments, respectively, that are the result of PCPS mediated recombination, of which 33 are in common. Overall, our results suggest that the tyrosine substitution facilitates the cleavage of the peptide, producing greater amounts of fragments that are mostly similar to pepvIII (FIG. 5D).

LC-MS/MS based sequence analysis of peptide products reveals multiple PCPS products capable of binding MHC Class I. While numerous fragments can be produced by proteasomal processing, the fragments that are presented by MHC Class I molecules are most relevant to CD8+ T cell activation. We determined which fragments bound to MHC Class I using a combination of HLA-immunoprecipitation (IP) and mass spectrometry analysis. Following proteasomal digestion of the parental peptides, the resulting fragments were passed over an HLA-IP column, and after washing, the remaining HLA-bound peptides were acid eluted and initially evaluated by MALDI-TOF. As demonstrated by the normalized overlaid spectra in FIG. 5E, both parental peptides produced a variety of fragments that bind to HLA Class I, but Y6-pepvIII digestion yielded a greater quantity of potentially antigenic peptide products (highlighted by red box in overlay).

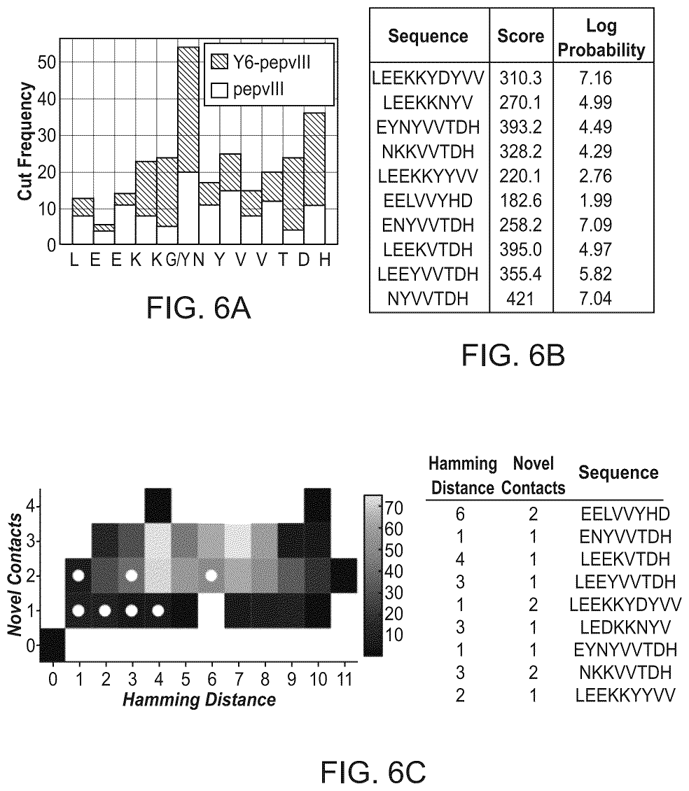

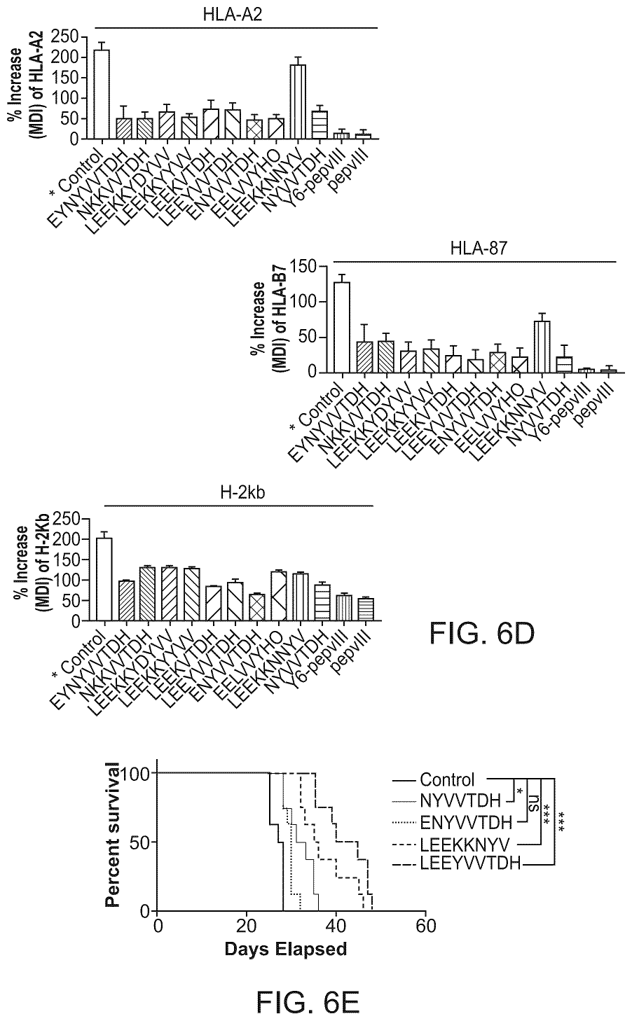

Next, we determined the sequence identity of the proteasome derived fragments that were bound to HLA Class I using LC-MS/MS combined with a database containing potential linear and PCPS fragments derived from pepvIII and Y6-pepvIII. From these sequences, we assessed the distribution and utilization of proteasomal cleavage sites across both parental peptides (FIG. 6a). While the profiles are overall similar, at each amino acid position the frequency of cleavage is higher for Y6-pepvIII than pepvIII, particularly at the G6Y substitution site. This confirms that this substitution not only enhances cleavage at this site but also increases the cleavage frequency at distal amino acid positions, as suggested by the computational predictions (FIG. 6a).

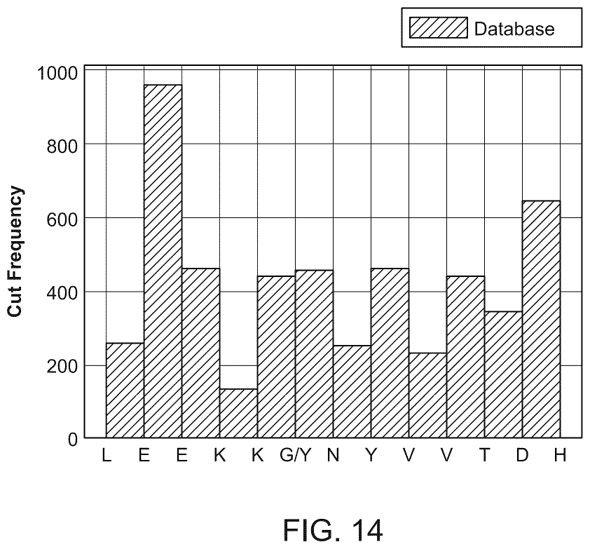

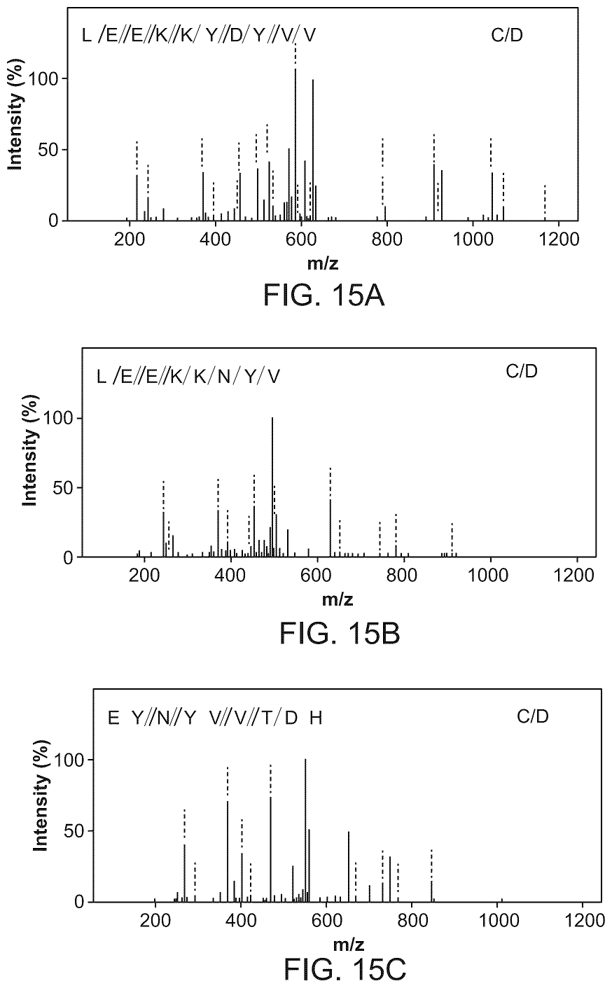

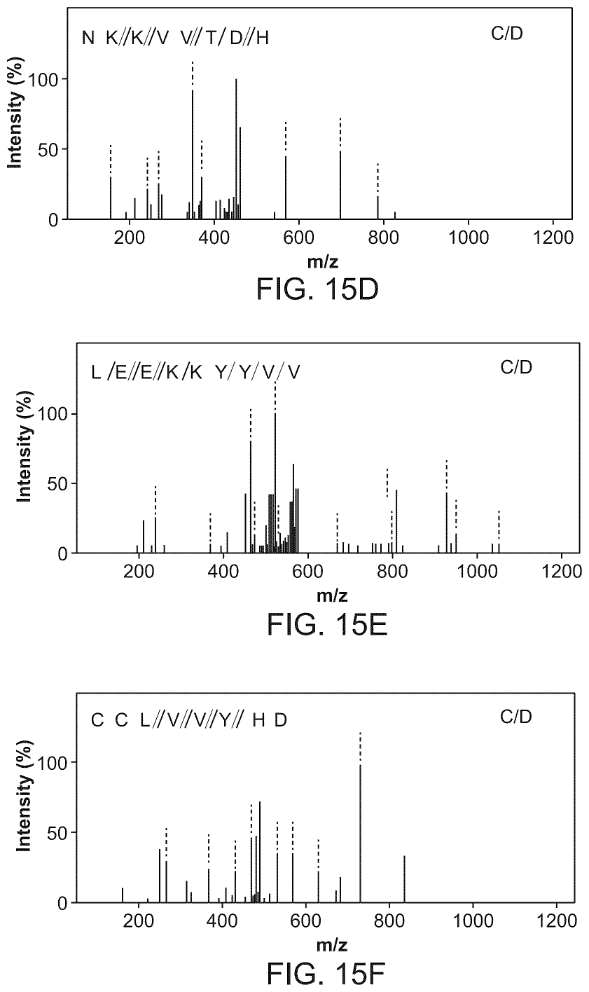

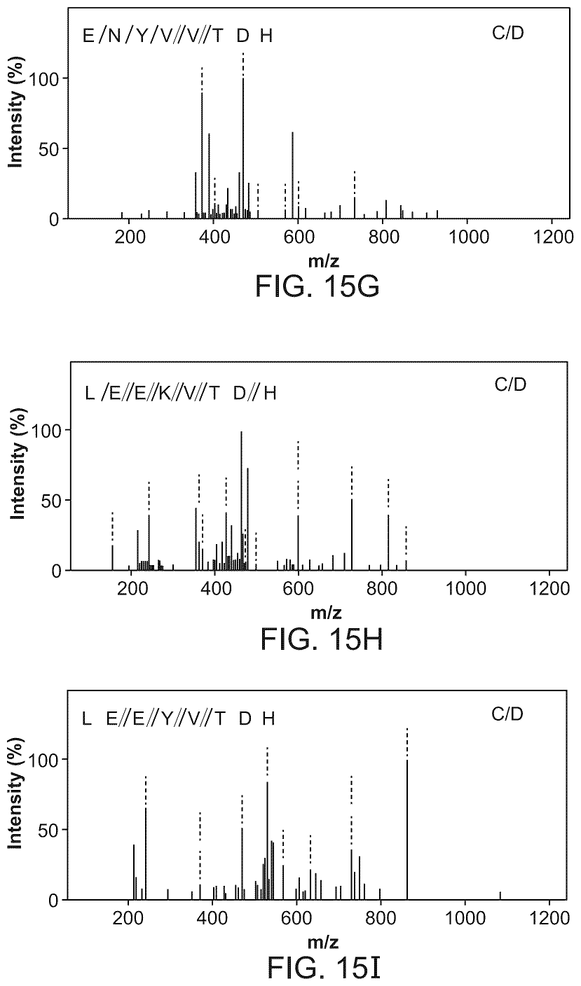

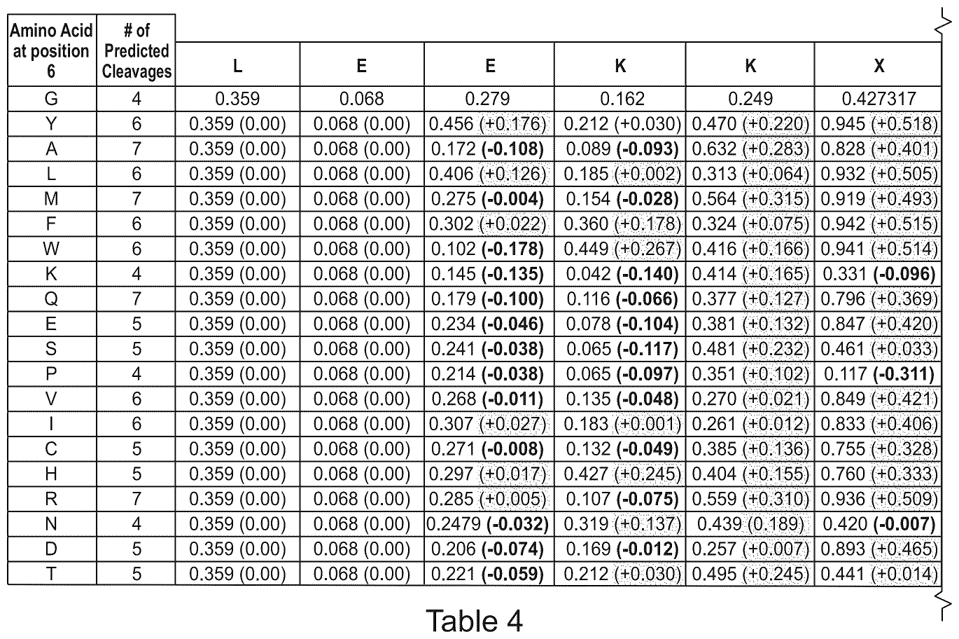

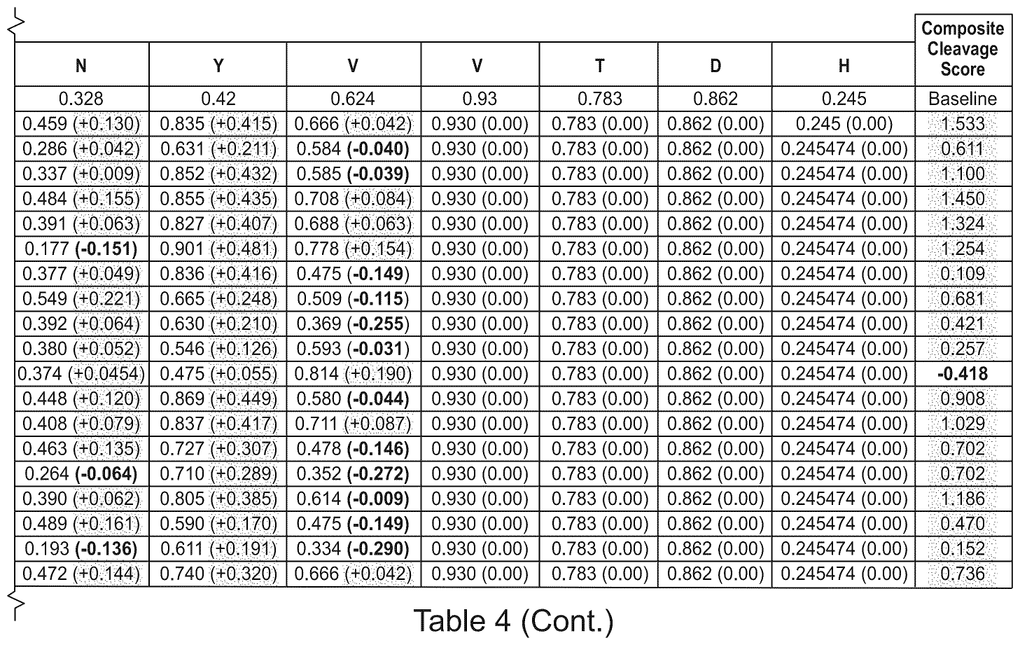

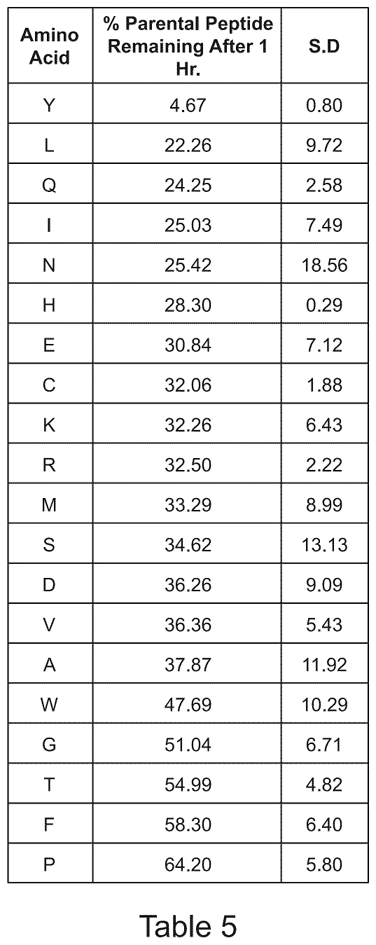

Importantly, this also shows that the sequence identity analysis and increase in cleavage site utilization is not an artifact of the database used, as the distribution of our reference library and the observed cut site distribution share little overlap (FIG. 14). To rigorously evaluate the returned peptides, we filtered the results of our LC-MS/MS analysis to only include peptides with absolute log probability exceeding 2 and Byonic score greater than 180 (see Materials and Methods). We identified a pool of 10 peptides that passed these quality thresholds and exhibited the appropriate size (7-12 amino acids) to be characterized as potential antigens (FIG. 6B). De novo sequencing was performed and each candidate was manually inspected to confirm that the major fragmented ions matched the identified sequences (FIG. 15). Unexpectedly, 9 of the sequences arose through PCPS and only one peptide was directly derived from EGFRvIII. It is important to note that each of these sequences can be found in both pepvIII and Y6-pepvIII. To assess the relatedness to the parental peptide and how these PCPS products arose, we calculated the Hamming distance and number of novel residue contacts for each of the PCPS derived peptides (FIG. 6C). The identified PCPS peptides retain homology to the parental sequence as indicated by the Hamming distance, and the re-ligation mechanism tends to involve 3 or fewer fragments.

The capacity of these products to be presented on MHC-class 1 molecules was confirmed in HLA/MHC stabilization assays. The ability of each candidate peptide to stabilize either human HLA-A2, HLA-B7 or mouse H-2Kb was evaluated in independent assays. As demonstrated in FIG. 6D, all tested products exhibited significant stabilization of HLA-A2 (top), HLA-B7 (middle) and H-2Kb (bottom) relative to either pepvIII or Y6-pepvIII. If these peptides bind to MHC Class I molecules, then vaccination with these peptides should show some improvement in survival. We evaluated the efficacy of four H-2kb binding peptides from the candidate pool as vaccination peptides in our intracranial glioma (GL261/vIII) model. Interestingly, vaccination with LEEKKNYV and LEEYVVTDH had a significant impact, increasing survival by 30% and 54% respectively, relative to KLH treated control mice (FIG. 6E). The one co-linear peptide derived from our LC-MS/MS analysis, NYVVTDH, induced an 18% increase in survival relative to controls. Despite not being co-linear with the EGFRvIII sequence, PCPS derived peptides can enhance survival in our model when used as an individual vaccine. Collectively, these results indicate that proteasomal digestion results in the production of multiple unique PCPS peptide products that can bind several MHC Class I alleles, implicating them as potential antigenic products with the ability to drive differential immune response profiles and improve survival as observed in our in vivo tumor models.

Glioblastoma is an invariably fatal tumor and progress towards a cure has been nominal. For other types of tumors, immunotherapy has sometimes resulted in dramatic increases in overall survival. EGFRvIII is a tumor specific and immunogenic receptor present in glioblastoma—as such, it represents a natural target for a peptide vaccine immunotherapeutic approach. While the phase Ill trial of pepvIII (ACT IV) failed to demonstrate a clear benefit, it is important to note that the sequence of the pepvIII peptide never underwent any sequence optimization prior to clinical investigation. Our study demonstrates that a single substitution to the original pepvIII amino acid sequence, aimed to increase proteasomal processing potential, can significantly increase survival and alter the dynamics of cellular anti-tumor immune responses. These results illustrate a potential new paradigm for the design of vaccines and justify the further development of Y6-pepvIII. We have completed pre-clinical studies and are currently planning an IND submission to the FDA to test Y6-pepvIII in a phase I trial for glioblastoma patients.

While there has been a recent focus on how administration route, adjuvants and checkpoint inhibitors can enhance the efficacy of peptide vaccines, much less attention has been paid to optimizing the vaccine for proteasomal processing. Yet, the processing of antigens may be key to enhancing the activity of vaccines in general. The proteasome is a critical component for the generation of the T cell response as it is responsible for processing proteins into antigenic peptides that will ultimately be presented to T cells in the context of MHC molecules. Beyond simply cleaving proteins into 8-11 amino antigenic peptides for presentation to T cells, it is now clear the proteasome has a major role in creating new antigens through peptide splicing to generate diverse non-contiguous antigenic peptides via PCPS. Because 20-30% of all peptides presented on a cell's surface are products of PCPS, this process may be highly relevant to T cell recognition of virally infected and malignantly transformed cells.

In general, PCPS is thought to be primarily determined by how frequently the proteasome cleaves a specific peptide bond. It is therefore reasonable to posit that a peptide that is more often cleaved would also more frequently undergo PCPS, thereby increasing the repertoire of antigenic peptides. By specifically altering the original pepvIII sequence to enhance proteasomal cleavage, we produced a peptide vaccine that was more extensively and effectively processed by the proteasome. Importantly, there have been numerous studies demonstrating that amino acid substitutions to peptide vaccines did not compromise T cell recognition of cognate antigens. Our findings reinforce this concept as vaccination with substituted Y6-pepvIII elicited antigen specific T cells that were capable of recognizing and killing EGFRvIII expressing target cells in vitro and T cell dependent tumor regression in vivo.

While maintaining the capacity to induce EGFRvIII specific immune responses, this specific alteration to the vaccinating peptide also resulted in greater production of potentially antigenic peptide products that demonstrate diversity in HLA binding. Using LC-MS/MS and novel computational methodologies, we discerned the sequence of ten candidate HLA binding peptides, nine of which are uniquely assembled by proteasomal processing. The presentation of multiple MHC Class I molecules likely contributes to shaping the immune response driven by this second-generation peptide vaccine. This concept was further supported by the observation that two of three identified PCPS peptides induced a significant increase in survival when administered as individual vaccination peptides. This result suggests that, despite not being co-linear with the targeted EGFRvIII antigen, PCPS derived peptides can induce immune responses against GL261/vIII cells and thus increasing survival in our model. While our study does not demonstrate that PCPS is solely responsible for the enhanced efficacy of the TYR-6 substituted vaccine, it does provide strong evidence that the spectrum of PCPS derived antigenic peptides contributes to the enhanced survival and unique immunologic response observed in our models. Our work raises the possibility that these products may in turn prove to be more effective vaccines.

An unexpected result was the differential induction of CD45+/CD3+/CD8+, CD45+/CD3+/CD4+/CD25+ and CD45+/CD11c+ immune cell populations and PD-1 expression in the TIL population by Y6-pepvIII over that seen with pepvIII. While it is possible that the single intact Y6-pepvIII peptide might elicit these effects, we speculate that here too, the increase in Y6-pepvIII derived peptides generated by the proteasome underlies this observation. This is additionally supported by the previously established direct association between proteasomal processing efficiency and T cell responses. The increased expression of PD-1 induced by Y6-pepvIII led to highly effective treatment with anti-PD-1 in the animal model. This is especially relevant because the expression of EGFRvIII in primary tumors is heterogeneous, leading to the possibility of antigen escape in vaccinated patients. Inhibiting PD-1 may allow T cell recognition of these escape variants. This further suggests that examining the expression of checkpoint molecules during the preclinical development process might suggest effective co-adjuvant therapies.

Increasing survival in glioblastoma will likely be achieved through a succession of improvements. By understanding the implications of proteasome cleavage and PCPS on antigen presentation and recognition on the surface of a tumor cell, we can refine the development of vaccines that will more comprehensively elicit T cell responses against tumor antigens. Overall, we believe that this work serves as a proof of principle model for how peptide vaccines can be optimized to enhance anti-tumor efficacy. Optimization may be as simple as identifying a substitution that increases the probability of proteasomal cleavage within the parental peptide, which then enhances subsequent PCPS. By combining structural modeling with proteasome prediction software, a similar approach can be employed to enhance the proteasomal processing of nearly any peptide vaccine. Moreover, these principles could be applied to the design of more effective vaccines for other diseases.

### Materials and Methods

Mice and cell lines. Animal protocols were approved by the Stanford University Research Compliance Office. 6 to 8-week-old C57BL/6J wild-type mice were housed and maintained in an approved facility at Stanford University. The GL261-wt cell line was obtained from the NIH and cultured in RPMI media (Caisson Labs) supplemented with 10% fetal bovine serum at 37° C. GL261/wt cells were derived from ATCC. These cells have been widely used in murine glioma models and are extensively characterized. To derive a GL261/vIII+ cell line, GL261/wt cells were stably transfected with a Tet-off Luciferase tagged EGFRvIII expression system (pRetroX-Tet-OFF (Clonetec #632105) and pRetroX-Tight-Pur EGFRvIII MSCV Luciferase PGK-hygro (Addgene #18782) by the Stanford Virus Core Facility to generate an EGFRvIII+ Luc+GL261 cell line (GL261/vIII). Expression of EGFRvIII was confirmed by both western blot and flow cytometry. The GL261/vIII cells were maintained in culture as described above with the addition of Puromycin (1 ug/mL), G418 (600 ug/mL) and Hygromycin (600 ug/mL). HC2 20d2/c cells were generated by stably transfecting NIH-3T3 cells (ATCC #: CRL-6441) with an EGFRvIII expression plasmid these cells were previously utilized to support an IND application for an anti-EGFRvIII vaccine. CT2A and CT2A/vIII were kindly provided by Prof. Luis-Sanchez Perez (Duke University). These cell lines were cultured in DMEM (high glucose) supplemented with 10% fetal bovine serum and L-Glutamine at 37° C. Expression of EGFRvIII (or lack thereof) was confirmed by flow cytometry.

Subcutaneous tumor model. 2×10⁶ EGFRvIII+HC2 cells were subcutaneously implanted into the hind flank of 6 to 8-week old mice as previously described (29). Tumors were first measured 4 days post tumor cell injection, and only recipient mice with detectable tumors at this time point were used for subsequent studies. Tumor bearing mice received 100 ug of KLH-conjugated amino acid substituted peptide vaccine emulsified at a 1:1 (v/v) ratio with Freund's Incomplete Adjuvant (Sigma-Aldrich). Mice were vaccinated at days 7 and 14 post tumor cell implantation; tumors were measured every 4 days and mice were followed for related morbidity. Morbidity was determined by a total tumor volume exceeding 2000 mm³ or by a tumor volume exceeding 1500 mm³ in combination with outward signs of morbidity such as consistently hunched posture and score of +2 or above on the grimace scale.

Intracranial model. Six to 8-week old C57BL/6J mice (Jackson Laboratories, Bar Harbor, ME), had gliomas established by stereotactically injecting 10,000 GL261/vIII of CT2A/vIII cells into the left striatum (2 mm posterior to the coronal suture and 2 mm lateral to the sagittal suture) on day 0 of the experimental process as previously described. Briefly, mice were anesthetized with isoflurane (4%) and placed in the stereotactic frame. An incision was made, and a burr hole was drilled over the striatum. GL261/vIII or CT2A/vIII cells were injected at the above stated coordinates using a Hamilton Microsyringe controlled by an automated micro-pump injection system. Mice were assigned to treatment groups randomly and tumor progression was monitored by bioluminescent signal as detected by the IVIS imaging system (Caliper Life Sciences, Hopkinton, MA). All glioma bearing mice were imaged at day 7 post implantation, only mice that exhibited a bioluminescent signal at this time point were included in experimental cohorts.

For survival experiments, each treatment group consisted of 6 to 15 mice. Control mice received 100 ug of unconjugated Keyhole Limpet Hemocyanin (KLH) emulsified at a 1:1 (v/v) ratio with Montanide ISA 51 VG (Seppic) via subcutaneous injection at day 0 (after tumor cell implantation), 7- and 14-days post tumor cell injection. All peptide vaccines were KLH conjugated, conjugated peptides were also emulsified at a 1:1 ratio with Montanide.

Experimental mice received 100 ug of emulsified peptide conjugate via subcutaneous injection on day 0 (after tumor cell implantation), 7 and 14 post tumor cell injection. Mice that survived beyond day 21 were imaged once again to assess tumor burden. For anti-PD-1 combination therapies, 100 ug of Montanide emulsified peptide-conjugate was administered via subcutaneous injection at day 0, 7 and 14, and 200 ug of anti-PD-1 antibody (Clone: RMP1-14, BioXcell) was administered via intraperitoneal injection at day 10, 14 and 18 (48). For survival studies using T cell depleted tumor bearing mice, 200 ug of anti-CD4 (Clone: GK1.5, BioXcell) or anti-CD8 (Clone: 53-6.7, BioXcell) was administered via intraperitoneal injection at day −2, 4, 7, 14 and 18 post tumor cell implantation; the peptide vaccination schedule remained unchanged. Morbidity was defined by consistent demonstration of outward indicators such as hunched posture, grimace score exceeding+2, impaired temperature control and ataxia. All moribund mice were imaged to confirm tumors based on a bioluminescent signal exceeding 1×10⁷ lumens.

Cytotoxicity assay. 6 to 8-week old C57BL/6J mice were vaccinated with 100 ug of KLH-conjugated peptide emulsified in Montanide, KLH alone in Montanide, or Montanide via subcutaneous injection every seven days for a total of 3 injections. Seven days after the third injection, T cells were isolated from the spleens of vaccinated mice using the EasySep Mouse CD90.2 Positive Selection Kit (Stem Cell Technologies, Vancouver BC). These isolated T cells were then co-cultured with pepvIII or Y6-pepvIII pulsed and interferon-gamma+Lipopolysaccharide activated DC2.4 cells for 7 days in the presence of IL-2 (100 ng/mL). After co-culture expansion, T cells were again purified from the T cell/DC2.4 (Millipore-sigma: SCC142) cell mixture using the EasySep Mouse CD8+ T Cell Isolation Kit (Stem Cell Technologies). These T cells were then co-cultured with 2×10⁴ GL261/vIII target cells at effector to target ratios of 0:1, 10:1 and 50:1 for six hours. Cytotoxic cell lysis was determined by measuring the percentage of 7AAD+ target cells by flow cytometry.

Tumor infiltrating lymphocyte isolation. Mice were lethally anesthetized for brain harvest at day 14 post tumor cell implantation. Tumors were resected from normal brain tissue and tumor infiltrating lymphocytes were extracted by tumor dissociation. Briefly, tumors were mechanically homogenized and incubated in Hanks Balanced Salt Solution (Corning) supplemented with 2% HEPES Buffer (Caisson Labs), DNase I (Sigma-Aldrich), and collagenase type IV (Worthington) at 37° C. The cell containing supernatant from this mixture was collected. Harvested cells were washed with and re-suspended in HBSS (+HEPES buffer). This cell suspension was filtered and layered on top of a 0.9M sucrose mixture and centrifuged to separate mononuclear cells from lipid debris carried over from the dissociation process. Purified cells were then washed and treated with ACK Lysis Buffer (Thermo-Fisher) to lyse RBCs.

Flow cytometry of tumor infiltrating lymphocytes. Isolated tumor infiltrating lymphocytes were washed with PBS supplemented with 2% fetal bovine serum and 10 mM EDTA. Cells were then evenly distributed and stained with an antibody panel to detect specific immune cell subsets. The CD4 T cell panel was as follows (all antibodies were obtained from BioLegend (San Diego, CA)): anti-mouse CD45 (30-F11), anti-mouse CD3 (17A2), anti-mouse CD4 (GK1.5), anti-mouse CD25 (C37) and anti-mouse PD-1 (29F.1A12) and 7-amino-actinomycin D (7AAD) to differentiate between live and dead cells. The CD8 Panel consisted of anti-mouse CD45 (30-F11), anti-mouse CD8 (53-5.8), anti-mouse PD-1 (29F.1A12) and 7AAD. A broad dendritic cell panel that included anti-mouse CD45 (30-F11), anti-mouse CD11c (3.9), and 7AAD, and finally and NK cell panel that included anti-mouse CD45 (30-F11), anti-mouse CD49b (DX5), anti-mouse CD161 (3.2.3) and 7AAD.

In vitro proteasome digestion. All peptides were synthesized by Lifetein LLC or ELIM Biopharm, reconstituted in molecular grade water (Fisher Scientific, 46000CV) or dimethyl sulfoxide (Sigma-Aldrich, D2650) and stored at −80° C. For the purposes of immunizing animals, peptides were synthesized with an additional cysteine at the C-terminus for conjugation to KLH, which was similarly done for pepvIII in the clinical trials. In vitro human 20s immunoproteasome digestions were performed at 37° C. using 2.0 ug of synthetic peptide, 1 ug of purified 20s immunoproteasome (South Bay Bio, SBB-PP0004) and a 10-molar excess of PA28 alpha (Boston Biochem, E-381-100) in 1×20s TEAD reaction buffer (Boston Biochem, B-80). The reaction was either quenched with 10% Trifluoroacetic acid (Thermo-fisher, PI-28904) or halted by ultra-centrifugation using an Amicon Ultra Centrifugal Unit (3 KDa) (Millipore, UFC501024) and used for mass spectrometry. Mass Spectrometry Analysis was done at the Stanford PAN Facility Stanford using the Perseptive Biosystems (ABI)-Voyager-DE RP-MALDI-TOF. Mass of the peptides were analyzed based on the Time of Flight needed for the ionized molecules to reach the detector which is a measure of the molecule's mass/charge ratio (m/z).

Immunoprecipitation of HLA peptides from proteasome digested pepvIII and Y6-pepvIII. IP columns were created following the protocol described in Pierce Crosslink IP kit (Thermo Scientific, 26147). Briefly, 500 ul of settled A/G Plus Agarose resin from the kit was washed and incubated with 1 mg of Anti-Human MHC Class 1 (HLA-A, HLA-B, HLA-C) antibody (W6/32 antibody from InVivo Mab, BE0079) overnight on a rotator at 4° C. An Epstein Barr Virus (EBV) immortalized B cell line (JY) was used as the HLA-A, B, C donor cell line. These were grown in 175 cm² cell culture flasks (Corning, 431079) to the confluency of 10\hat{ }9 cells. Donor cells were washed pelleted and lysed according to Pierce protocols. Cell Lysate was precleared and incubated with agarose resin and antibody at 4° C. The following day, the column (Lysate+Resin+Antibody) was centrifuged, washed and treated with DSS crosslinker. To remove endogenous peptides, the column was washed with Citrate Phosphate Buffer (pH 3.0) for 60 seconds and immediately flushed with wash buffer and centrifuged to remove the eluate. 200 μg of digested peptide (pepvIII and Y6-pepvIII) products 5 μg/mL of human β2-micrglobulin (Lee BioSolutions, 126-11-1) were then added to the column and incubated one ice on overnight. Finally, the column was washed, and bound peptides products were eluted with citrate phosphate buffer (pH 3.0). Eluates were zip tipped using Pierce C18 tips (Pierce, 87784) and analyzed by MALDI-TOF or LC-MS/MS.

Identification of PCPS peptide and MHC Class I binding predictions. Amino acid sequences and predicted binding to HLA-A*201 molecules were identified using the IEDB database and the BIMAS dissociation half-life prediction algorithm. Values from the proteasome digests of peptides were refined using Mascot Distiller (Matrix Sciences). These peaks were then compared to the reference of all known linear fragments derived from either pepvIII or Y6-pepvIII using the STRAIN program yielding the peaks that were co-linear or derived via PCPS. LC-MS/MS was used to more robustly evaluate a pool of candidate peptides for potential antigenic fragments. Proteasome-digested pepvIII and Y6-pepvIII samples were analyzed on a Thermo Orbitrap Fusion nanoLC/MS, fragmented using collision-induced dissociation (CID) in the ion trap. To perform a peptide-spectra match (PSM) search, we aggregated molecular weights observed through MALDI-TOF and calculated each linear and PCPS-derived arrangement of parental sequence that matched the original weight. To focus our search on functionally relevant potential HLA-binding peptides, we restricted our library to peptides of lengths between 8 and 11 residues. We used a Python implementation of this algorithm to generate a Fasta database of 871 candidate recombination peptides. Using the Byonic v3.6.0 software, the database was evaluated against observed spectra, and PSMs were selected through filtering with absolute log probability threshold of 2 and Byonic score of 180. To perform scoring, Byonic first calculates a protein p-value using a simple probabilistic model based on decoy, reversed sequence peptides and subsequently outputs the absolute value of the log base 10 of this p-value which is the likelihood of that the peptide spectra matches arising due to random chance. Further implementation details are available in the Byonic User Manual and supporting publication.

The minimum edit distance, or Hamming distance, between each candidate PCPS peptide and cumulative peptide library was then calculated using Python to assess homology to the parental peptide. Sequences with a lower Hamming distance are more similar in sequence to Y6-pepvIII. Number of ligations (or number of novel contacts between residues created through PCPS-mediated ligation), which captures the number of constituent parental fragments, and Hamming distance were chosen as two axes to evaluate recombination complexity.

T2 binding assay. T2 (ATCC CRL 1992; TAP-deficient; HLA-A*201), B7 and RMA-S cell lines were washed with serum free RPMI-1640 medium supplemented with 25 mM HEPES and plated at a concentration of 3×10 in a 24-well plate. The cells were incubated for 12 hours with 25 ug/mL synthetic peptide in the presence of 20 ug/mL of β2-microglobulin in serum free RPMI 1640 medium supplemented with 25 mM HEPES at 37° C. The cells were washed, blocked and stained with an HLA-A2 specific monoclonal antibody (BB7.2) (Abcam ab27728) conjugated to FITC prior to flow cytometry analysis on the NovoCyte 2000 (ACEA Bioscience Inc.)

Statistical Analysis. Data was analyzed by two-tailed Student t test or ANOVA using the GraphPad (La Jolla, CA) Prism 7 software. Survival was analyzed by the Kaplan-Meier method and relevant groups were prepared by log-rank tests. Comparisons were presented as mean±SEM and any values of p<0.05 were considered significant. * denotes p<0.05, ** denotes p<0.01, *** denotes p<0.001 and **** denotes p<0.0001.

### REFERENCES AND NOTES

R. Stupp et al., Effects of radiotherapy with concomitant and adjuvant temozolomide versus radiotherapy alone on survival in glioblastoma in a randomised phase Ill study: 5-year analysis of the EORTC-NCIC trial. Lancet Oncol 10, 459-466 (2009).
M. Preusser et al., Current concepts and management of glioblastoma. Ann Neurol 70, 9-21 (2011).
Q. T. Ostrom et al., CBTRUS statistical report: primary brain and central nervous system tumors diagnosed in the United States in 2007-2011. Neuro Oncol 16 Suppl 4, iv1-63 (2014).
P. Y. Wen, S. Kesari, Malignant gliomas in adults. N Engl J Med 359, 492-507 (2008).
D. K. Moscatello et al., Frequent Expression of a Mutant Epidermal Growth Factor Receptor in Multiple Human Tumors. Cancer Research 55, 5536 (1995).
C. A. Del Vecchio et al., EGFRvIII gene rearrangement is an early event in glioblastoma tumorigenesis and expression defines a hierarchy modulated by epigenetic mechanisms. Oncogene 32, 2670-2681 (2013).
C. E. Pelloski et al., Epidermal growth factor receptor variant Ill status defines clinically distinct subtypes of glioblastoma. J Clin Oncol 25, 2288-2294 (2007).
D. R. Emlet et al., Targeting a glioblastoma cancer stem-cell population defined by EGF receptor variant III. Cancer Res 74, 1238-1249 (2014).
D. A. Chistiakov, I. V. Chekhonin, V. P. Chekhonin, The EGFR variant Ill mutant as a target for immunotherapy of glioblastoma multiforme. Eur J Pharmacol 810, 70-82 (2017).
D. M. O'Rourke et al., A single dose of peripherally infused EGFRvIII-directed CAR T cells mediates antigen loss and induces adaptive resistance in patients with recurrent glioblastoma. Sci Transl Med 9, (2017).
T. Kumai, H. Kobayashi, Y. Harabuchi, E. Celis, Peptide vaccines in cancer—old concept revisited. Current opinion in immunology 45, 1-7 (2017).
C. A. Del Vecchio, G. Li, A. J. Wong, Targeting EGF receptor variant Ill: tumor-specific peptide vaccination for malignant gliomas. Expert Rev Vaccines 11, 133-144 (2012).
M. Weller et al., Rindopepimut with temozolomide for patients with newly diagnosed, EGFRvIII-expressing glioblastoma (ACT IV): a randomised, double-blind, international phase 3 trial. Lancet Oncol 18, 1373-1385 (2017).
M. R. Neagu, D. A. Reardon, Rindopepimut vaccine and bevacizumab combination therapy: improving survival rates in relapsed glioblastoma patients? Immunotherapy 7, 603-606 (2015).
J. A. Berzofsky, M. Terabe, L. V. Wood, Strategies to use immune modulators in therapeutic vaccines against cancer. Semin Oncol 39, 348-357 (2012).
J. D. Buhrman, J. E. Slansky, Improving T cell responses to modified peptides in tumor vaccines. Immunol Res 55, 34-47 (2013).
J. S. Blum, P. A. Wearsch, P. Cresswell, Pathways of antigen processing. Annu Rev Immunol 31, 443-473 (2013).
D. A. Ferrington, D. S. Gregerson, Immunoproteasomes: structure, function, and antigen presentation. Prog Mol Biol Transl Sci 109, 75-112 (2012).
E. J. Sijts, P. M. Kloetzel, The role of the proteasome in the generation of MHC class I ligands and immune responses. Cell Mol Life Sci 68, 1491-1502 (2011).
C. R. Berkers et al., Definition of Proteasomal Peptide Splicing Rules for High-Efficiency Spliced Peptide Presentation by MHC Class I Molecules. J Immunol 195, 4085-4095 (2015).
N. Vigneron et al., An Antigenic Peptide Produced by Peptide Splicing in the Proteasome. Science 304, 587 (2004).
J. Liepe et al., A large fraction of HLA class I ligands are proteasome-generated spliced peptides. Science 354, 354-358 (2016).
R. C. Landry et al., Antibody recognition of a conformational epitope in a peptide antigen: Fv-peptide complex of an antibody fragment specific for the mutant EGF receptor, EGFRvIII. J Mol Biol 308, 883-893 (2001).
M. K. McCarthy, J. B. Weinberg, The immunoproteasome and viral infection: a complex regulator of inflammation. Frontiers in Microbiology 6, 21 (2015).
M. B. Winter et al., Immunoproteasome functions explained by divergence in cleavage specificity and regulation. eLife 6, e27364 (2017).
A. Dalet, N. Vigneron, V. Stroobant, K. Hanada, B. J. Van den Eynde, Splicing of distant peptide fragments occurs in the proteasome by transpeptidation and produces the spliced antigenic peptide derived from fibroblast growth factor-5. J Immunol 184, 3016-3024 (2010).
M. Mishto et al., Driving forces of proteasome-catalyzed peptide splicing in yeast and humans. Mol Cell Proteomics 11, 1008-1023 (2012).
N. Vigneron et al., An antigenic peptide produced by peptide splicing in the proteasome. Science 304, 587-590 (2004).
D. K. Moscatello, G. Ramirez, A. J. Wong, A naturally occurring mutant human epidermal growth factor receptor as a target for peptide vaccine immunotherapy of tumors. Cancer Res 57, 1419-1424 (1997).
T. Szatmari et al., Detailed characterization of the mouse glioma 261 tumor model for experimental glioblastoma therapy. Cancer Sci97, 546-553 (2006).
H. I. Cho, S. H. Jung, H. J. Sohn, E. Celis, T. G. Kim, An optimized peptide vaccine strategy capable of inducing multivalent CD8(+) T cell responses with potent antitumor effects. Oncoimmunology 4, el043504 (2015).
K. R. Jordan, R. H. McMahan, C. B. Kemmler, J. W. Kappler, J. E. Slansky, Peptide vaccines prevent tumor growth by activating T cells that respond to native tumor antigens. Proc Natl Acad Sci USA 107, 4652-4657 (2010).
B. Huang et al., Advances in Immunotherapy for Glioblastoma Multiforme. J Immunol Res 2017, U.S. Pat. No. 3,597,613 (2017).
C. M. Jackson, M. Lim, Immunotherapy for Glioblastoma: Playing Chess, Not Checkers. Clin Cancer Res 24, 4059-4061 (2018).
M. Lim, Y. Xia, C. Bettegowda, M. Weller, Current state of immunotherapy for glioblastoma. Nat Rev Clin Oncol 15, 422-442 (2018).
M. Preusser, M. Lim, D. A. Hafler, D. A. Reardon, J. H. Sampson, Prospects of immune checkpoint modulators in the treatment of glioblastoma. Nat Rev Neurol 11, 504-514 (2015).
S. T. Garber et al., Immune checkpoint blockade as a potential therapeutic target: surveying CNS malignancies. Neuro Oncol 18, 1357-1366 (2016).
A. H. Wu et al., Identification of EGFRvIII-derived CTL epitopes restricted by HLA A0201 for dendritic cell based immunotherapy of gliomas. J Neurooncol 76, 23-30 (2006).
F. Ebstein et al., Proteasomes generate spliced epitopes by two different mechanisms and as efficiently as non-spliced epitopes. Sci Rep 6, 24032 (2016).
A. C. M. Platteel et al., Multi-level Strategy for Identifying Proteasome-Catalyzed Spliced Epitopes Targeted by CD8(+) T Cells during Bacterial Infection. Cell Rep 20, 1242-1253 (2017).
A. K. Bentzen et al., T cell receptor fingerprinting enables in-depth characterization of the interactions governing recognition of peptide-MHC complexes. Nat Biotechnol, (2018).
M. J. Ciesielski et al., Antitumor cytotoxic T-cell response induced by a survivin peptide mimic. Cancer Immunol Immunother 59, 1211-1221 (2010).
R. A. Fenstermaker et al., Clinical study of a survivin long peptide vaccine (SurVaxM) in patients with recurrent malignant glioma. Cancer Immunol Immunother 65, 1339-1352 (2016).
I. Dekhtiarenko et al., Peptide Processing Is Critical for T-Cell Memory Inflation and May Be Optimized to Improve Immune Protection by CMV-Based Vaccine Vectors. PLoS Pathog 12, e1006072 (2016).
K. Textoris-Taube et al., The T210M Substitution in the HLA-a*02:01 gp100 Epitope Strongly Affects Overall Proteasomal Cleavage Site Usage and Antigen Processing. J Biol Chem 290, 30417-30428 (2015).

H. Modjtahedi et al., Targeting of cells expressing wild-type EGFR and type-Ill mutant EGFR (EGFRvIII) by anti-EGFR MAb ICR62: a two-pronged attack for tumour therapy. Int J Cancer 105, 273-280 (2003).
J. Zeng et al., Anti-PD-1 blockade and stereotactic radiation produce long-term survival in mice with intracranial gliomas. Int J Radiat Oncol Biol Phys 86, 343-349 (2013).
T. Garzon-Muvdi et al., Dendritic cell activation enhances anti-PD-1 mediated immunotherapy against glioblastoma. Oncotarget 9, 20681-20697 (2018).
V. Jurtz et al., NetMHCpan-4.0: Improved Peptide-MHC Class I Interaction Predictions Integrating Eluted Ligand and Peptide Binding Affinity Data. J Immunol 199, 3360-3368 (2017).
M. Bern, Y. J. Kil, C. Becker, Byonic: advanced peptide and protein identification software. Curr Protoc Bioinformatics Chapter 13, Unit 13 20 (2012).

The atomic coordinates for the pepvIII and Y6-pepvIII models are available in the Model Archive database under the archive numbers ma-7c64k and ma-5s4ct respectively. The mass spectrometry proteomics data has been deposited to the ProteomeXchange Consortium via the PRoteomics IDEntifications (PRIDE) repository (Submission #: 1-20190227-11204).

TABLE 7
Top 20 NR database hits

| | % ID | | | | | % ID |
|---|---|---|---|---|---|---|
| | to | | SEQ | Closest | SEQ | to |
| | KF11 | | ID | Library | ID | Library |
| GI number | GAG | NR Peptide | NO | Hit (>60%) | NO | Peptide |
| 302335486 | 35.7 | RSLAPEVRGYW | 415 | KSLTPEVRGYW | 435 | 81.8 |
| 345792467 | 42.9 | WISSPEIRAVE | 416 | WISHPEIRAYF | 436 | 81.8 |
| 495145889 | 28.6 | ASSRPELALAY | 417 | ASFRPELALRY | 437 | 81.8 |
| 459942335 | 35.7 | WISHPEIKAAF | 418 | WISHPEIRAYF | 436 | 81.8 |
| 430749919 | 42.9 | RSLKPEVREVE | 419 | KSLTPEVREYF | 438 | 72.7 |
| 494716083 | 42.9 | ASLRPEVREAF | 420 | KSLAPEVRELF | 439 | 72.7 |
| 493030958 | 42.9 | KSLYPEIREVE | 421 | RSFYPEIREYF | 440 | 72.7 |
| 497464005 | 28.6 | LSGVPEIRERW | 422 | LSLRPEIREYW | 441 | 72.7 |
| 497193348 | 35.7 | LTIRPEIRPRW | 423 | GTIRPEIREMW | 442 | 72.7 |
| 488856804 | 42.9 | ASFKPELPDFF | 424 | NSFKPEIPDYF | 443 | 72.7 |
| 430004692 | 35.7 | STISPEIRLEW | 425 | GTISPEIREMW | 444 | 72.7 |
| 471573742 | 42.9 | ASLKPEVPLVE | 426 | LSLRPEVPLFF | 445 | 72.7 |
| 495156089 | 42.9 | SSGAPEVRELF | 427 | SSGVPEVRMME | 446 | 72.7 |
| 301092772 | 35.7 | SSVVPELPMAF | 428 | SSVVPEVRMMF | 447 | 72.7 |
| 348664816 | 42.9 | RSFYPELRLLF | 429 | RSFYPEIREYF | 440 | 72.7 |
| 497177556 | 50.0 | LTISPEIPPYF | 430 | GTIRPEIPDYF | 448 | 72.7 |
| 497797312 | 42.9 | ESFRPEIRQYE | 431 | RSFYPEIREYF | 440 | 72.7 |
| 448510490 | 50.0 | GSLSPELRPIF | 432 | LSGSPELRMIF | 449 | 72.7 |
| 15790131 | 35.7 | STLSPELRGRW | 433 | SSFSPELRMRW | 450 | 72.7 |
| 313682157 | 42.9 | KSFRPELKEFY | 434 | ASFRPELAEFW | 451 | 72.7 |

### Example 2

### Method for Rapid Identification of CD8+ T Cell Epitopes

A method is provided for the rapid identification of CD8+ T cell epitopes. For smaller proteins/peptides (i.e., less than 50 amino acids), the protein is incubated with activated 20S Immunoproteasome with a10 fold molar excess of PA28 activator alpha subunit protein in 1×20S TEAD buffer at 37 C for 24 hrs.

However, whether in vitro or in cells, highly ordered structures or intact proteins are poor substrates for the proteasome due to the inaccessibility of internal cleavage sites and there needs to be a disordered region to initiate degradation. In early experiments, we found no proteasome degradation for large and intact proteins such as KLH or the fusion protein for the RBD region in the Spike protein of SARS-CoV-2. As such, for larger proteins, we have discovered that denaturation of the protein allows for efficient degradation by the proteasome.

To make the sites available for cleavage, the protein is first denatured with 8 M Urea for 2-3 hrs in 50 mM Tris-HCl at 37 C and then the digestion mixture is diluted to 1 M Urea in 50 mM Tris-HCl followed by the proteasome digestion protocol above. This enabled the digestion of intact proteins without requiring synthesis of individual portions.

Next, the proteasome digest undergoes immunoprecipitation (IP) with HLA molecules. The IP column is created following the protocol described in Pierce Crosslink IP kit. Briefly, 500 ul of settled A/G Plus Agarose resin from the kit was washed and incubated with 1 mg of Anti-Human MHC Class 1 (HLA-A, HLA-B, HLA-C) antibody (W6/32 antibody) overnight on a rotator at 4° C. An Epstein Barr Virus (EBV) immortalized B cell line (JY) is used as the source of HLA-A, B, C Class I molecules. These are grown in 175 cm2 cell culture flasks to the confluency of 10¹ cells. Donor cells are washed, pelleted and lysed according to Pierce protocols. Cell Lysate is precleared and incubated with agarose resin and antibody at 4° C. The following day, the column (Lysate+Resin+Antibody) is centrifuged, washed and treated with DSS crosslinker. To remove endogenous peptides, the column was washed with Citrate Phosphate Buffer (pH 3.0) for 60 seconds and immediately flushed with wash buffer and centrifuged to remove the eluate. 200 mg of proteasome digested products and 5 mg/mL of human b2-micrglobulin are then added to the column and incubated on ice overnight. Finally, the column is washed, and bound peptides products are eluted with citrate phosphate buffer (pH 3.0). Eluates are zip tipped using Pierce C18 tips (Pierce, 87784) to remove salts and the peptides eluted and then analyzed by either MALDI-ToF or LC-MS/MS for de novo sequencing.

### Example 3

Providing a database to perform the de novo sequencing. For de novo sequencing to be successful, the sequence to be identified must be in the database scanned. The computer attempts to match the molecular weight as determined by mass spectrometry from the b- and y-series of ions from a particular fragment with the same molecular weights from the known sequences in the database. If there is a reasonable match across the b- and y-series it will infer a match to that known sequence, but it cannot efficiently infer the sequence of a collection of peptides that are otherwise unknown. Because we start with only one protein in this analysis, the co-linear (also called contiguous) peptides with the parent sequence are efficiently identified. However, most studies underestimate the number of PCPS fragments as the construction of these databases make certain assumptions regarding length, intervening sequence length, and length of the recombining fragments.

To be comprehensive, we have developed 3 different approaches to PCPS database construction: A. The first approach is very efficient for PCPS across a relatively short (˜13 amino acid) distance. To obtain a peptide-spectra match (PSM) search, we aggregate the molecular weights observed through MALDI-TOF and calculate each linear and PCPS-derived arrangement of the parental sequence that matches the original weight. To focus our search on functionally relevant potential HLA-binding peptides, we restrict our library to peptides of lengths between 8 and 11 residues. We then use a Python implementation of this algorithm to generate a FASTA database of co-linear and PCPS recombination peptides.

B. For larger proteins, we use the MW data from the MALDI-ToF to obtain the sequences for the co-linear fragments in the digest. These represent a collection of various length fragments. We are most interested in those that are approximately 2 amino acids up to 12 aa. The fragments from 8 to 12 aa represent co-linear fragments directly derived from the parental peptide that can bind to MHC-I (while some might also represent PCPS products, the goal here is to identify the sequence of pieces co-linear with the protein). The fragments from 2 to 10 aa are used to construct our database of possible PCPS recombinants. Since we know the sequence of the parental peptide, we can use a combination of mMass and Prospector software, which takes into account ionization status, Na+ ions, and other potential modifications to determine the sequence of co-linear fragments between 200 to 1400 Da. Since mass spectrometry relies on MW for sequence identification, it is possible for smaller fragments that multiple sequences could produce the same MW fragment. If necessary, we can resolve this through additional analysis of the ionization series in separate MS/MS. Once the sequence of the 2-12 aa pieces is known, we can construct a database that encompasses potential PCPS fragments. Fragments that are between 2-10 aa are used to construct the PCPS database. It has also been reported that cis-ligation of fragments within a protein can occur up to 40 aa, so we have chosen 50 aa as the farthest distance apart we will consider each fragment. There is evidence that trans-ligation can occur during the PCPS process, i.e., fragments from two distinct proteins can be ligated together. However, using isotope labeled short fragments, we did not find evidence for this in our study of EGFRvIII. Our work on a 2^{nd} generation EGFRvIII vaccine also showed that no PCPS were composed of more than 3 fragments, although the pieces could be assembled in any order. Also, since 99% of MHC-I binding peptides are between 8-12 amino acids, that is the smallest and largest overall potential binding peptide size we will use consider. Theoretically, isoleucine and leucine present a problem in MS/MS based de novo sequencing because they have identical molecular weights—this is not a problem in making our database because we are using the known protein sequence to assemble our theoretical PCPS fragments. The code that has already been written in Python is adapted to assemble this database where the 2-10 aa fragments across any given 50 aa window are used in combinatorial fashion to make hypothetical PCPS sequences of between 8-12 aa but containing no more than 3 fragments.

C. Because PCPS database construction in the first two examples requires experimental identification of fragments, we wished to develop an algorithm dependent only on the protein sequence. Here, all possible windows of from 2 to 12 aa's across a sequence of about 50 aa's are generated. This list of sequences is used to generate all possible recombinations of from 1 to 3 fragments that range from 8-12 aa, Redundant sequences will be pruned. To continue walking across the entire protein sequence, peptide windows containing the first amino acid will be eliminated and windows including the next amino acid in the sequence will be added. Because the vast majority of recombinations will be the same, it is only necessary to contemplate the recombinations arising from the new amino acid. In practice this generates an enormous database of ˜2 Gb per 70 aas and requires significant computing power for proteins of >400 aas. Thus, this strategy is used to create a universal database where a protein sequence can be plugged in generate the actual fragments. While not reducing the database size, this will significantly reduce the computational power required.

Once the co-linear and PCPS fragments that bind to HLA-1 are identified, they can be confirmed for binding to the appropriate HLA-1 or MHC-1 in an MHC stabilization assay. Further activity can be explored in an appropriate biologic assay. For example, for those fragments identified from the EGFRvIII peptide used as an anti-tumor vaccine, it can be determined if these peptides induce CTL activity and anti-tumor activity against cells expressing EGFRvIII. For those fragments derived from proteins of the SARS-CoV-2 virus, it can be seen if these peptides elicit antibodies, elicit CTL activity, if they bind CD8+ T cells from COVID-19 patients, and if they confer protection for animals or humans against infection by the SARS-CoV-2 virus when used as a vaccine.

### Example 4

A method to enhance the proteasomal cleavage of a given peptide such that it increases the production of co-linear and PCPS fragments, either in in vitro assays or when given to a human or an animal as a vaccine or when expressed in cells using a cDNA construct. For the anti-EGFRvIII tumor vaccine known as pepvIII (SEQ ID 413: LEEKKGNYVVTDH), we used the protein structure of pepvIII and realized that there was a β-turn in the molecule at Gly-6 which formed a hairpin in the structure. Through successive amino acid substitution, it was determined that tyrosine yielded the highest proteasomal fragmentation of this peptide resulting in higher yields of co-linear and PCPS fragments that bound to MHC-1 and HLA-1.

The utility of mutating to enhance proteasome cleavage resulted in the tyrosine substituted peptide (called Y6-pepvIII) demonstrating significantly greater anti-tumor activity than the original pepvIII.

Provided is a simple means to enhance the efficacy of any vaccine, either against infectious agents or to treat cancers, by scanning the structure of the proteins within the vaccine for β-turns, then mutating the amino acid at the center of the β-turn to tyrosine, then use such tyrosine substituted protein as the basis for the next version of the vaccine.

### Example 5

Sequences identified from the 13 aa pepvIII vaccine that are co-linear or PCPS fragments. Using the methods elaborated in Part 1 and 2, we identified sequences that resulted from proteasomal cleavage and bound to both MHC-I and HLA-1. Nine of these peptides are PCPS products, while one is co-linear with pepvIII. 4 of these peptides were tested as anti-tumor vaccines in mice bearing intracranial tumors expressing EGFRvIII.

TABLE 1A
SEQ ID NO: 1. EYNYVVTDH
SEQ ID NO: 2. NKKVVTDH
SEQ ID NO: 3. LEEKKYDYVV
SEQ ID NO: 4. LEEKKYYVV
SEQ ID NO: 5. LEEKVTDH
SEQ ID NO: 6. LEEYVVTDH
SEQ ID NO: 7. ENYVVTDH
SEQ ID NO: 8. EELVVYHD
SEQ ID NO: 9. LEEKKNYV
SEQ ID NO: 10. NYVVTDH

Because the unique epitope found in this 13 amino acid region of EGFRvIII could recombine with sequences outside this region, we applied the methods in Part 1 and 2 to a protein containing the extracellular domain of the EGFRvIII protein. The following PCPS peptides were found to arise between sequences found in the EGFRvIII related deletion joined to other parts of the extracellular domain:

TABLE 1B
PCPS Peptides

| | |
|---|---|
| SEQ ID NO: 11 | CGALSILEEK |
| SEQ ID NO: 12 | DGYVVTDH |
| SEQ ID NO: 13 | DIGIGLEEK |
| SEQ ID NO: 14 | DLYVVTDH |
| SEQ ID NO: 15 | DSLVVTDHGEK |
| SEQ ID NO: 16 | DYVVTDH |
| SEQ ID NO: 1 | EEKKNGYVVTDH |
| SEQ ID NO: 18 | EMVVTDH |
| SEQ ID NO: 19 | GCTSVVTDH |
| SEQ ID NO: 20 | GDYVVTDH |
| SEQ ID NO: 21 | GGNYVVTDH |
| SEQ ID NO: 22 | GLEEKKNCTGVR |
| SEQ ID NO: 23 | GLEEKMEKHFK |
| SEQ ID NO: 24 | GNEKCTGADSY |
| SEQ ID NO: 25 | GNGVYVVTDH |
| SEQ ID NO: 26 | GNYSYEMEEDHG |
| SEQ ID NO: 27 | GVCVVTDH |
| SEQ ID NO: 28 | GVVYVVTDH |
| SEQ ID NO: 29 | HGSCVRLSIEEK |
| SEQ ID NO: 30 | INATNYVVTDH |
| SEQ ID NO: 31 | KDGYVVTD |
| SEQ ID NO: 32 | KEEGIYVVTDH |
| SEQ ID NO: 33 | KGDYVVTDH |
| SEQ ID NO: 34 | KGGENYVVTDH |
| SEQ ID NO: 35 | KGIGEFKD |
| SEQ ID NO: 36 | KGIGIGEFTS |
| SEQ ID NO: 37 | LESINNYV |
| SEQ ID NO: 38 | LSINYKKGK |
| SEQ ID NO: 39 | MEEKGEKK |
| SEQ ID NO: 40 | NGYVVTDH |
| SEQ ID NO: 41 | NYVVTDA |
| SEQ ID NO: 42 | NYVVTTDH |
| SEQ ID NO: 43 | VVNIKNYVVT |
| SEQ ID NO: 44 | KGNDHVTDH |
| SEQ ID NO: 45 | KGNHFKNCVR |
| SEQ ID NO: 46 | KGNLEIKNI |
| SEQ ID NO: 47 | KKDGYVVTD |
| SEQ ID NO: 48 | KKGNYVVT |
| SEQ ID NO: 49 | KNGYVVTD |
| SEQ ID NO: 50 | KVVTDHGSK |
| SEQ ID NO: 51 | LEEESINNATNIK |
| SEQ ID NO: 52 | LEEGVRKCK |
| SEQ ID NO: 53 | LEEKGKNYV |
| SEQ ID NO: 54 | KGNDHVTDH |
| SEQ ID NO: 55 | LEEKGKNYVVT |
| SEQ ID NO: 56 | LEEKGKNYVVTDH |
| SEQ ID NO: 57 | LEEKIGEYVVTD |
| SEQ ID NO: 58 | LEEKIGEYVVTDH |
| SEQ ID NO: 59 | LEEKKGI |
| SEQ ID NO: 60 | LEEKKGISGDLR |
| SEQ ID NO: 61 | LEEKKVCCK |
| SEQ ID NO: 62 | LEEKKVCGVR |
| SEQ ID NO: 63 | LEEKNATNIKN |
| SEQ ID NO: 64 | LEEKTSISNIK |
| SEQ ID NO: 65 | LEEKVCGVRK |
| SEQ ID NO: 66 | LEEVVMEEDGVR |
| SEQ ID NO: 67 | LEKKEGNYVVTDH |
| SEQ ID NO: 68 | LEKKNGYVVTDH |
| SEQ ID NO: 69 | LELEVTDYVVTDH |
| SEQ ID NO: 70 | NYVVVTDH |
| SEQ ID NO: 71 | SLSINKGNYEK |
| SEQ ID NO: 72 | SLSINNYVV |
| SEQ ID NO: 73 | TDIGEFLEK |
| SEQ ID NO: 74 | TSIGVKKGG |
| SEQ ID NO: 75 | TSIVTDHGSCVR |

### Example 6

Peptides that bind to HLA-I identified using the methods elaborated in Part 1 and 2 that are derived from the SARS-CoV-2 virus. First shown are sequences from the receptor binding domain (RBD) of the spike glycoprotein from the SARSCoV-2 virus.

TABLE 2A

| | |
|---|---|
| SEQ ID NO: 76 | ADSFVIGDEVR |
| SEQ ID NO: 77 | APGQTGKIA |
| SEQ ID NO: 78 | DSFVIRGDEV |
| SEQ ID NO: 79 | DSKVGGNYNY |
| SEQ ID NO: 80 | GFQPTNGVGY |
| SEQ ID NO: 81 | GVGYQPYRV |
| SEQ ID NO: 82 | GVGYQPYRVV |
| SEQ ID NO: 83 | IAPGQTGKI |
| SEQ ID NO: 84 | IAPGQTGKIAD |
| SEQ ID NO: 85 | KVGGNYNY |
| SEQ ID NO: 86 | KVGGNYNYL |
| SEQ ID NO: 87 | NDSFVIRGDEV |
| SEQ ID NO: 88 | NLDSKVGGNYN |
| SEQ ID NO: 89 | NLDSKVGGNYNY |
| SEQ ID NO: 90 | NPGQTGKIADY |
| SEQ ID NO: 91 | NYKLPDDF |
| SEQ ID NO: 92 | RDISTEIY |
| SEQ ID NO: 93 | RDISTEIYQ |
| SEQ ID NO: 94 | SKVGGNYNY |
| SEQ ID NO: 95 | VGYQPYRV |
| SEQ ID NO: 96 | VQPTESIPFGEK |
| SEQ ID NO: 97 | ADSFVIRGDEV |
| SEQ ID NO: 98 | ADYSVLYN |
| SEQ ID NO: 99 | APGQTGKIADY |
| SEQ ID NO: 100 | DISTEIYQA |
| SEQ ID NO: 101 | DSKVGGNYN |
| SEQ ID NO: 102 | ELLHAPATV |
| SEQ ID NO: 103 | DSKVGGNYNYL |
| SEQ ID NO: 104 | GDEVRQIAPGQ |
| SEQ ID NO: 105 | RDISTEIYQA |
| SEQ ID NO: 106 | RQIAPGQTG |
| SEQ ID NO: 107 | RQIAPGQTGKI |
| SEQ ID NO: 108 | SFVIRGDEV |
| SEQ ID NO: 109 | SPTKLNDL |
| SEQ ID NO: 110 | TNLCPFRIS |
| SEQ ID NO: 111 | VGYQPYRVV |
| SEQ ID NO: 112 | VIRGDEVRQI |
| SEQ ID NO: 113 | VQPTLYFSTFK |
| SEQ ID NO: 114 | YADSFVIR |
| SEQ ID NO: 115 | YGFQPTNGV |
| SEQ ID NO: 116 | YGFQPTNGVGY |
| SEQ ID NO: 117 | YGVSPTKL |

The methods in Part 1 and 2 were applied to the entire Si domain of the Spike protein of SARS-CoV-2. The following peptides that arise from POPS were identified:

TABLE 2B
PCPS Peptides

| | |
|---|---|
| SEQ ID NO: 118 | AAGYLQPRTF |
| SEQ ID NO: 119 | ASTGSNVFQTR |
| SEQ ID NO: 120 | ASVSPTKL |
| SEQ ID NO: 121 | AVLYQGVNC |
| SEQ ID NO: 122 | AVLYQGVNCTE |
| SEQ ID NO: 123 | AVLYQGVNCTEV |
| SEQ ID NO: 124 | AVRDPQTI |
| SEQ ID NO: 125 | DASYQTQTNSPR |
| SEQ ID NO: 126 | DATNVVIKV |
| SEQ ID NO: 127 | DIITPCSF |
| SEQ ID NO: 128 | DLEGKQGNFKIN |
| SEQ ID NO: 129 | DLEGKQGNFKNI |
| SEQ ID NO: 130 | DPLSETKCTI |
| SEQ ID NO: 131 | DSKVGGNYNYYR |
| SEQ ID NO: 132 | DTGSNVFQTR |
| SEQ ID NO: 133 | DTSNQVAVL |
| SEQ ID NO: 134 | DVSVITPGTNTS |
| SEQ ID NO: 135 | EEHVNNSYE |
| SEQ ID NO: 136 | ERDISTIYQ |
| SEQ ID NO: 137 | ESTGSNVFQTR |
| SEQ ID NO: 138 | EVPVAIHADAGI |
| SEQ ID NO: 139 | FAGVSPTK |
| SEQ ID NO: 140 | FASVLYN |
| SEQ ID NO: 141 | GETRFASVY |
| SEQ ID NO: 142 | GPDKVFR |
| SEQ ID NO: 143 | GRPQTLEIL |
| SEQ ID NO: 144 | GSNVFQTAR |
| SEQ ID NO: 145 | GTQDLFL |
| SEQ ID NO: 146 | GTTLDSKTQSI |
| SEQ ID NO: 147 | GVGYQPLHAP |
| SEQ ID NO: 148 | GVNCTEVPVAIH |
| SEQ ID NO: 149 | GVSPTKLL |
| SEQ ID NO: 150 | GVTPGTNTSNQV |
| SEQ ID NO: 151 | IADSNNLDSK |
| SEQ ID NO: 152 | IGAEIPVASQ |
| SEQ ID NO: 153 | IGAEPIGASVA |
| SEQ ID NO: 154 | ISNSFSTF |
| SEQ ID NO: 155 | ISNSFSTFK |
| SEQ ID NO: 156 | ITPGTNTSQDV |
| SEQ ID NO: 157 | KLFVIFT |
| SEQ ID NO: 158 | KSNIVVI |
| SEQ ID NO: 159 | LDISTEIY |
| SEQ ID NO: 160 | LDISTEIYQA |
| SEQ ID NO: 161 | LLIVNIK |
| SEQ ID NO: 162 | LLSGEVF |
| SEQ ID NO: 163 | LSIGEVF |
| SEQ ID NO: 164 | LTDIADTTDAVRD |
| SEQ ID NO: 165 | LTTRTQPPAY |
| SEQ ID NO: 166 | LTVGEVF |
| SEQ ID NO: 167 | LVDLPIR |
| SEQ ID NO: 168 | LVDLQTL |
| SEQ ID NO: 169 | NDSFVIR |
| SEQ ID NO: 170 | NGLLHAPATV |
| SEQ ID NO: 171 | NGLTGITPGTNTSN |
| SEQ ID NO: 172 | NIDINLVR |
| SEQ ID NO: 173 | NIDTPINL |
| SEQ ID NO: 174 | NLDPLSETKC |
| SEQ ID NO: 175 | NLREDLPI |
| SEQ ID NO: 176 | NLREEPLV |
| SEQ ID NO: 177 | NLTTRTQLPSSVL |
| SEQ ID NO: 178 | NLVFRSDGVYF |
| SEQ ID NO: 179 | NPVLPIR |
| SEQ ID NO: 180 | NSNNLSNL |
| SEQ ID NO: 181 | NSNNLSNLK |
| SEQ ID NO: 182 | NVRDPQTL |
| SEQ ID NO: 183 | PGTNTSNQPV |
| SEQ ID NO: 184 | PVAIHADQLV |
| SEQ ID NO: 185 | PVAIHADV |
| SEQ ID NO: 186 | QEHVNNSYEC |
| SEQ ID NO: 187 | QGSNVFQ |
| SEQ ID NO: 188 | QGSNVFQTR |
| SEQ ID NO: 189 | QGVNCTEVPVAI |
| SEQ ID NO: 190 | QGVNCTEVPVAIH |
| SEQ ID NO: 191 | QGVNCTEVPVAIHA |
| SEQ ID NO: 192 | QLPIGINI |
| SEQ ID NO: 193 | QQSVITPGT |
| SEQ ID NO: 194 | QQSVITPGTN |
| SEQ ID NO: 195 | QSLLVVI |
| SEQ ID NO: 196 | QSTGSNVF |
| SEQ ID NO: 197 | QSTGSNVFQTR |
| SEQ ID NO: 198 | QTGKIAF |
| SEQ ID NO: 199 | QTITPGTNTSNQV |
| SEQ ID NO: 200 | QTPGTNTN |
| SEQ ID NO: 201 | QTQTNSPRAR |
| SEQ ID NO: 202 | SCDIPIGAGI |
| SEQ ID NO: 203 | SFGGVSVITPGTD |
| SEQ ID NO: 204 | SFVIGDEVR |
| SEQ ID NO: 205 | SGVEKGIYQT |
| SEQ ID NO: 206 | SGVEKGIYQTS |
| SEQ ID NO: 207 | SIVNNATNVV |
| SEQ ID NO: 208 | SKTPINLVR |
| SEQ ID NO: 209 | SLEKGIYQTSNF |
| SEQ ID NO: 210 | SNIIRTLD |
| SEQ ID NO: 211 | SSEFRVY |
| SEQ ID NO: 212 | STGSNVFQHH |
| SEQ ID NO: 213 | STGSNVFQTAR |
| SEQ ID NO: 214 | SYQTQTNSH |
| SEQ ID NO: 215 | SYVSQPFL |
| SEQ ID NO: 216 | SYVSQPFLM |
| SEQ ID NO: 217 | TESNVRDPQTL |
| SEQ ID NO: 218 | TEVPVAIHAN |
| SEQ ID NO: 219 | TGSNVFQTAR |
| SEQ ID NO: 220 | TGSNVFTR |
| SEQ ID NO: 221 | TKADSFVIR |
| SEQ ID NO: 222 | TLPGTNTSNQV |
| SEQ ID NO: 223 | TPGNTSNQVAVL |
| SEQ ID NO: 224 | TPGTAVL |
| SEQ ID NO: 225 | TPGTNTAVLY |
| SEQ ID NO: 226 | TQLPHSTQD |
| SEQ ID NO: 227 | TQLPPAHVSG |
| SEQ ID NO: 228 | TQLPPAYTD |
| SEQ ID NO: 229 | TQLPSSVL |
| SEQ ID NO: 230 | TQSLLIQF |
| SEQ ID NO: 231 | TQSLLIVNI |
| SEQ ID NO: 232 | TQSLLIVNIK |
| SEQ ID NO: 233 | TQSLLVY |
| SEQ ID NO: 234 | TSFTVEK |
| SEQ ID NO: 235 | TWSNVFQT |
| SEQ ID NO: 236 | TWSNVFQTR |
| SEQ ID NO: 237 | TWSNVFQTRAG |
| SEQ ID NO: 238 | TWSNVFQTRAGC |
| SEQ ID NO: 239 | VAIGSNVF |
| SEQ ID NO: 240 | VAPGQTGKI |
| SEQ ID NO: 241 | VCGVDTTDAV |
| SEQ ID NO: 242 | VGGNYNYLLK |
| SEQ ID NO: 243 | VGKSNLKPFE |
| SEQ ID NO: 244 | VNLTTPAYTNSFT |
| SEQ ID NO: 245 | VNLTTPFFSNV |
| SEQ ID NO: 246 | VPVAIHADAGI |
| SEQ ID NO: 247 | VQPTSVTF |
| SEQ ID NO: 248 | VSPTKLNDLF |
| SEQ ID NO: 249 | WIGAEHVNN |
| SEQ ID NO: 250 | WSTGSNVFQTR |
| SEQ ID NO: 251 | YADSFVIGDEVR |
| SEQ ID NO: 252 | YQGVNCTEVPVAI |
| SEQ ID NO: 253 | YQGVNCTEVPVAIH |

Shown below are the peptides from the nucleocapsid protein of SARS-CoV-2 that bind to HLA-I.

TABLE 3

| | |
|---|---|
| SEQ ID NO: 254 | NKDGIIWV |
| SEQ ID NO: 255 | NSTPGSSR |
| SEQ ID NO: 256 | VTPSGTWL |
| SEQ ID NO: 257 | QRNAPRITF |
| SEQ ID NO: 258 | GLPYGANKD |
| SEQ ID NO: 259 | ANKDGIIWV |
| SEQ ID NO: 260 | HIGTRNPAN |
| SEQ ID NO: 261 | PANNAAIVL |
| SEQ ID NO: 262 | LDRLNQLES |
| SEQ ID NO: 263 | QQGQTVTKK |
| SEQ ID NO: 264 | GDQELIRQG |
| SEQ ID NO: 265 | TDYKHWPQI |
| SEQ ID NO: 266 | EVTPSGTWL |
| SEQ ID NO: 267 | KTFPPTEPK |
| SEQ ID NO: 268 | QTVTLLPAA |
| SEQ ID NO: 269 | DLDDFSKQL |
| SEQ ID NO: 270 | RPQGLPNNTA |
| SEQ ID NO: 271 | TQHGKEDLKF |
| SEQ ID NO: 272 | HIGTRNPANN |
| SEQ ID NO: 273 | QQGQTVTKKS |
| SEQ ID NO: 274 | GRRGPEQTQG |
| SEQ ID NO: 275 | RGPEQTQGNF |
| SEQ ID NO: 276 | QGNFGDQELI |
| SEQ ID NO: 277 | QELIRQGTDY |
| SEQ ID NO: 278 | GTDYKHWPQI |
| SEQ ID NO: 279 | TDYKHWPQIA |
| SEQ ID NO: 280 | MEVTPSGTWL |
| SEQ ID NO: 281 | KDPNFKDQVI |
| SEQ ID NO: 282 | PNFKDQVILL |
| SEQ ID NO: 283 | KFPRGQGVPIN |
| SEQ ID NO: 284 | HIGTRNPANNA |
| SEQ ID NO: 285 | RNPANNAAIVL |
| SEQ ID NO: 286 | PQGTTLPKGFY |
| SEQ ID NO: 287 | GNGGDAALALL |
| SEQ ID NO: 288 | LLLLDRLNQLE |
| SEQ ID NO: 289 | QQGQTVTKKSA |
| SEQ ID NO: 290 | RGPEQTQGNFG |
| SEQ ID NO: 291 | QGTDYKHWPQI |
| SEQ ID NO: 292 | DDKDPNFKDQV |
| SEQ ID NO: 293 | DETQALPQRQK |
| SEQ ID NO: 294 | KQQTVTLLPAA |
| SEQ ID NO: 295 | DLDDFSKQLQQ |
| SEQ ID NO: 296 | NGPQNQRNAPRI |
| SEQ ID NO: 297 | ALNTPKDHIGTR |
| SEQ ID NO: 298 | PKDHIGTRNPAN |
| SEQ ID NO: 299 | QLPQGTTLPKGF |
| SEQ ID NO: 300 | NSSRNSTPGSSR |
| SEQ ID NO: 301 | GNGGDAALALLL |
| SEQ ID NO: 302 | NQLESKMSGKGQ |
| SEQ ID NO: 303 | GRRGPEQTQGNF |
| SEQ ID NO: 304 | RGPEQTQGNFGD |
| SEQ ID NO: 305 | RQGTDYKHWPQI |
| SEQ ID NO: 306 | KDPNFKDQVILL |
| SEQ ID NO: 307 | ADETQALPQRQK |
| SEQ ID NO: 308 | LDDFSKQLQQSM |

### Example 7

The V600E mutation in B-Raf is present in ˜70% of human melanomas and also numerous other types of cancer. Vaccines based on the unique sequences present in the mutant protein can provide the basis for anti-tumor therapy; or the analysis of CD8+ T cell responses as part of monitoring therapy. We have applied the methods in Part 1 and 2 to the full length wild type human B-Raf and also a full length protein containing the V600E mutation. Because the V600E mutation alters the 3 dimensional structure of the protein, it also alters the cleavage of proteins by the proteasome. We have compiled the following list of peptides in Tables 4A (co-linear peptides)-4B (POPS peptides) that are specifically found in B-Raf proteins with the V600E mutation by our methods:

TABLE 4A
Co-linear Peptide Epitopes

| | |
|---|---|
| SEQ ID NO: 309 | SGGGGGGAEPGQALF |
| SEQ ID NO: 310 | ASMDTVTSSSSSSL |
| SEQ ID NO: 311 | NVTAPTPQQLQAFK |
| SEQ ID NO: 312 | SIGPQILTSPSPSK |
| SEQ ID NO: 313 | GYLSPDLSKVRSN |
| SEQ ID NO: 314 | HVEVLENVPLTTH |
| SEQ ID NO: 315 | NVTAPTPQQLQAF |
| SEQ ID NO: 316 | GDFGLATEKSRW |
| SEQ ID NO: 317 | GGEHNPPSIYLE |
| SEQ ID NO: 318 | PKIHRSASEPSL |
| SEQ ID NO: 319 | RGYLSPDLSKVR |
| SEQ ID NO: 320 | SIPIPQPFRPAD |
| SEQ ID NO: 321 | SNPKSPQKPIVR |
| SEQ ID NO: 322 | SSSSLSVLPSSL |
| SEQ ID NO: 323 | VLENVPLTTHNF |
| SEQ ID NO: 324 | DTVTSSSSSSL |
| SEQ ID NO: 325 | FQNPTDVARSN |
| SEQ ID NO: 326 | HHPIPQEEASL |
| SEQ ID NO: 327 | KGKWHGDVAVK |
| SEQ ID NO: 328 | LSPDLSKVRSN |
| SEQ ID NO: 329 | NINNRDQIIFM |
| SEQ ID NO: 330 | PSLNRAGFQTE |
| SEQ ID NO: 331 | RIGSGSFGTVY |
| SEQ ID NO: 332 | TAPTPQQLQAF |
| SEQ ID NO: 333 | TVVPARCGVTV |
| SEQ ID NO: 334 | ALQQREQQLL |
| SEQ ID NO: 335 | ASIELLARSL |
| SEQ ID NO: 336 | DQLDLLFVSK |
| SEQ ID NO: 337 | GLSATPPASL |
| SEQ ID NO: 338 | GNGTDFSVSS |
| SEQ ID NO: 339 | IGDFGLATEK |
| SEQ ID NO: 340 | INTIEPVNID |
| SEQ ID NO: 341 | NNRDQIIFMV |
| SEQ ID NO: 342 | NVPLTTHNFV |
| SEQ ID NO: 343 | QAFKNEVGVL |
| SEQ ID NO: 344 | QKSPGPQRER |
| SEQ ID NO: 345 | SSLSVLPSSL |
| SEQ ID NO: 346 | STKPQLAIVT |
| SEQ ID NO: 347 | SVFQNPTDVA |
| SEQ ID NO: 348 | TGEELHVEVL |
| SEQ ID NO: 349 | VLENVPLTTH |
| SEQ ID NO: 350 | GDFGLATEK |
| SEQ ID NO: 351 | KLIDIARQT |
| SEQ ID NO: 352 | LSPDLSKVR |
| SEQ ID NO: 353 | SIELLARSL |
| SEQ ID NO: 354 | TSPSPSKSI |
| SEQ ID NO: 355 | TVKIGDFGL |
| SEQ ID NO: 356 | ENVPLTTH |
| SEQ ID NO: 357 | KNEVGVLR |
| SEQ ID NO: 358 | LTSPSPSK |
| SEQ ID NO: 359 | MAPEVIRM |
| SEQ ID NO: 360 | NRDQIIFM |
| SEQ ID NO: 361 | RGYLSPDL |
| SEQ ID NO: 362 | SPDLSKVR |
| SEQ ID NO: 363 | SPGPQRER |
| SEQ ID NO: 364 | AQGMDYL |
| SEQ ID NO: 365 | DLLFVSK |
| SEQ ID NO: 366 | FLPNKQR |
| SEQ ID NO: 367 | HIEALLD |
| SEQ ID NO: 368 | HVNILLF |
| SEQ ID NO: 369 | IDIARQT |
| SEQ ID NO: 370 | LASIELL |
| SEQ ID NO: 371 | PDLSKVR |
| SEQ ID NO: 372 | RDQIIFM |
| SEQ ID NO: 373 | RPLFPQI |
| SEQ ID NO: 374 | SIELLAR |
| SEQ ID NO: 375 | SVLPSSL |
| SEQ ID NO: 376 | VFLPNKQ |

TABLE 4B
PCPS PEPTIDES

| | |
|---|---|
| SEQ ID NO: 377 | TALSGSSPSAPASDSI |
| SEQ ID NO: 378 | AALSGGGGGGAEPSP |
| SEQ ID NO: 379 | MAALSGGGAGAGAGA |
| SEQ ID NO: 380 | TGTPPASLPGSLTNV |
| SEQ ID NO: 381 | TTGPPASLPGSLTNV |
| SEQ ID NO: 382 | SAMDTVTSSSSSSL |
| SEQ ID NO: 383 | TALSGSSPSAPASD |
| SEQ ID NO: 384 | VTSSSSSVLPSSL |
| SEQ ID NO: 385 | ASVLPSSLSPQK |
| SEQ ID NO: 386 | GTVVTAPTPQQL |
| SEQ ID NO: 387 | LSATPPASPGSL |
| SEQ ID NO: 388 | SAPKTPIQAGGY |
| SEQ ID NO: 389 | SAVLPSSLSPQK |
| SEQ ID NO: 390 | SAVLSNPKSPQK |
| SEQ ID NO: 391 | TDISWLTGVEVL |
| SEQ ID NO: 392 | TVGQRGSFGTVY |
| SEQ ID NO: 393 | ALQKSPGPQER |
| SEQ ID NO: 394 | HVYSTKPQLAI |
| SEQ ID NO: 395 | IGPQILTSPSR |
| SEQ ID NO: 396 | LNVTAPTPQLQ |
| SEQ ID NO: 397 | NNTIEPVNIDD |
| SEQ ID NO: 398 | SVLPSSLSVPK |
| SEQ ID NO: 399 | HINTIEPVDL |
| SEQ ID NO: 400 | INNRDQIFMV |
| SEQ ID NO: 401 | IQDGEKKPIS |
| SEQ ID NO: 402 | DERPLFPQL |
| SEQ ID NO: 403 | FVRFFTLAF |
| SEQ ID NO: 404 | NVPLTTNFH |
| SEQ ID NO: 405 | RDEPLFPQI |
| SEQ ID NO: 406 | EKPVEVLE |
| SEQ ID NO: 407 | IGDFGLSR |
| SEQ ID NO: 408 | IKQIEALL |
| SEQ ID NO: 409 | KARGLIPE |
| SEQ ID NO: 410 | KLASIELL |
| SEQ ID NO: 411 | LSGLFPAI |
| SEQ ID NO: 412 | HQLLASL |

1. A method for the identification of peptide sequences from a polypeptide of interest that are presented on Class I MHC proteins, the method comprising:
incubating a polypeptide of interest with activated 20S immunoproteasome and a molar excess of PA28 activator alpha subunit protein for a period of time sufficient to digest the polypeptide, wherein candidate polypeptides of greater than about 50 kD are pre-treated by denaturation, to generate a proteasome digest;
immunoprecipitating the proteasome digest with Class I MHC proteins by incubation with a substrate comprising immobilized HLA proteins;
washing the substrate free of unbound peptides;
eluting the bound peptides;
analyzing the eluted peptides for molecular weight by mass spectrometry;
identifying the sequence of the eluted peptides by de novo sequencing using tandem mass spectrometry through matching the molecular weight to a reference database.

2. The method of claim 1, wherein the polypeptide of interest is a cancer antigen.

3. The method of claim 1, wherein the polypeptide of interest is an autoimmune antigen.

4. The method of claim 2, wherein the polypeptide of interest is EGFRvIII.

5. The method of claim 1, wherein the polypeptide of interest is a pathogen antigen.

6. The method of claim 5, wherein the pathogen antigen is SARS-CoV2 spike protein or nucleocapsid protein.

7. The method of claim 2, wherein identifying the sequence of eluted peptides is performed by matching the molecular weight with the same molecular weights from known sequences in the reference database.

8. The method of claim 7, wherein molecular weights observed through MALDI-TOF are aggregated; and each linear and PCPS-derived arrangement of the parental sequence that matches the original weight is calculated, restricted to peptides of lengths between about 8 and about 11 amino acid residues in length to generate a FASTA database of co-linear and PCPS spliced peptides.

9. The method of claim 7, wherein molecular weight data from MALDI-ToF is used to generate a database of co-linear fragments in a digest, an algorithm is used to assemble a database where 2-10 aa fragments across any given 50 aa window are used in combinatorial fashion to make hypothetical PCPS sequences of between 8-12 aa, containing no more than 3 fragments.

10. The method of claim 7, wherein an algorithm is used to assemble a database where all possible 2-12 aa fragments across any given 50 aa window are used in combinatorial fashion to make hypothetical PCPS sequences of between 8-12 aa, containing no more than 3 fragments.

11. The method of claim 1, further comprising confirming a peptide for binding to an appropriate class I MHC in an MHC stabilization assay.

12. The method of claim 1, further comprising confirming a peptide by a functional assay.

13. A method for enhancing proteasomal cleavage of a polypeptide antigen by sequence modification, in order to increase production of co-linear and PCPS fragments, wherein amino acid residues that create a hairpin in the structure of a protein antigen are modified to remove or replace the residue with a tyrosine.

14. The method of claim 13, wherein glycine present at residue 6 of the EGFRvIII tumor vaccine, SEQ ID 413 LEEKKGNYVVTDH, is replaced with tyrosine to enhance presentation of the antigen to T cells.

15. A peptide antigen selected from: an EGFRvIII peptide antigen as set forth in Table 1A or Table 1B; a SARS-CoV2 spike protein peptide antigen as set forth in Table 2A or Table 2B: a SARS-CoV2 nucleocapsid protein peptide antigen as set forth in Table 3; and a human V600E BRAF peptide antigen as set forth in Table 4A or 4B.

16-18. (canceled)

19. A peptide antigen according to claim 15, wherein the peptide is a proteasome-catalyzed peptide splicing product.

20. An immunogenic composition comprising a peptide of claim 15.

21. A method of immunizing an individual, comprising administering an effective dose of an immunogenic composition of claim 20.
